SEQUENCE LISTING (395 sequences)

SEQ ID NO: 1 - protein, 127 aa, Artificial Sequence
  Synthetic polypeptide-alpha-EGFR1 sdAb
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 2 - protein, 10 aa, Artificial Sequence
  Synthetic polypeptide-alpha-EGFR1 sdAb sdCDR1
GRTFSSYAMG

SEQ ID NO: 3 - protein, 16 aa, Artificial Sequence
  Synthetic polypeptide-alpha-EGFR1 sdAb sdCDR2
INWSSGSTYYADSVKG

SEQ ID NO: 4 - protein, 18 aa, Artificial Sequence
  Synthetic peptide-alpha-EGFR1 sdAb sdCDR3
GYQINSGNYNFKDYEYDY

SEQ ID NO: 5 - protein, 124 aa, Artificial Sequence
  Synthetic polypeptide-alpha-EGFR2 sdAb
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 6 - protein, 10 aa, Artificial Sequence
  Synthetic polypeptide-alpha-EGFR2 sdAb sdCDR1
GRTSRSYGMG

SEQ ID NO: 7 - protein, 17 aa, Artificial Sequence
  Synthetic polypeptide-alpha-EGFR2 sdAb sdCDR2
GISWRGDSTGYADSVKG

SEQ ID NO: 8 - protein, 15 aa, Artificial Sequence
  Synthetic polypeptide-alpha-EGFR2 sdAb sdCDR3
AAGSAWYGTLYEYDY

SEQ ID NO: 9 - protein, 127 aa, Artificial Sequence
  Synthetic polypeptide-h-alpha-EGFR1 sdAb
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSS

SEQ ID NO: 10 - protein, 10 aa, Artificial Sequence
  Synthetic polypeptide-h-alpha-EGFR1 sdAb sdCDR1
GRTFSSYAMG

SEQ ID NO: 11 - protein, 16 aa, Artificial Sequence
  Synthetic polypeptide-h-alpha-EGFR1 sdAb sdCDR2
INWSSGSTYYADSVKG

SEQ ID NO: 12 - protein, 18 aa, Artificial Sequence
  Synthetic polypeptide-h-alpha-EGFR1 sdAb sdCDR3
GYQINSGNYNFKDYEYDY

SEQ ID NO: 13 - protein, 124 aa, Artificial Sequence
  Synthetic polypeptide-aEGFR2a sdAb
EVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 14 - protein, 10 aa, Artificial Sequence
  Synthetic polypeptide-aEGFR2a sdAb sdCDR1
GRTSRSYGMG

SEQ ID NO: 15 - protein, 17 aa, Artificial Sequence
  Synthetic polypeptide-aEGFR2a sdAb sdCDR2
GISWRGDSTGYADSVKG

SEQ ID NO: 16 - protein, 15 aa, Artificial Sequence
  Synthetic polypeptide-aEGFR2a sdAb sdCDR3
AAGSAWYGTLYEYDY

SEQ ID NO: 17 - protein, 124 aa, Artificial Sequence
  Synthetic polypeptide-h-alpha-EGFR2d sdAb
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 18 - protein, 10 aa, Artificial Sequence
  Synthetic polypeptide-h-alpha-EGFR2d sdAb sdCDR1
GRTSRSYGMG

SEQ ID NO: 19 - protein, 17 aa, Artificial Sequence
  Synthetic polypeptide-h-alpha-EGFR2d sdAb sdCDR2
GISWRGDSTGYADSVKG

SEQ ID NO: 20 - protein, 15 aa, Artificial Sequence
  Synthetic polypeptide-h-alpha-EGFR2d sdAb sdCDR3
AAGSAWYGTLYEYDY

SEQ ID NO: 21 - protein, 114 aa, Artificial Sequence
  Synthetic polypeptide-alpha-FOLR1 h77-2 sdAb
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 22 - protein, 10 aa, Artificial Sequence
  Synthetic polypeptide-alpha-FOLR1 h77-2 sdAb sdCDR1
GFTVSNSVMA

SEQ ID NO: 23 - protein, 16 aa, Artificial Sequence
  Synthetic polypeptide-alpha-FOLR1 h77-2 sdAb sdCDR2
IINSIGITNYADSVKG

SEQ ID NO: 24 - protein, 6 aa, Artificial Sequence
  Synthetic polypeptide-alpha-FOLR1 h77-2 sdAb sdCDR3
NFDRIY

SEQ ID NO: 25 - protein, 113 aa, Artificial Sequence
  Synthetic polypeptide-alpha-FOLR1 h59.3 sdAb
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSS

SEQ ID NO: 26 - protein, 10 aa, Artificial Sequence
  Synthetic polypeptide-alpha-FOLR1 h59.3 sdAb sdCDR1
GNTFSISAMG

SEQ ID NO: 27 - protein, 16 aa, Artificial Sequence
  Synthetic polypeptide-alpha-FOLR1 h59.3 sdAb sdCDR2
VTHSDYSTNYADSVKG

SEQ ID NO: 28 - protein, 5 aa, Artificial Sequence
  Synthetic polypeptide-alpha-FOLR1 h59.3 sdAb sdCDR3
YGIDY

SEQ ID NO: 29 - protein, 117 aa, Artificial Sequence
  Synthetic polypeptide-alpha-FOLR1 h22-4 sdAb
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSS

SEQ ID NO: 30 - protein, 10 aa, Artificial Sequence
  Synthetic polypeptide-alpha-FOLR1 h22-4 sdAb sdCDR1
GTTFSRDVMG

SEQ ID NO: 31 - protein, 16 aa, Artificial Sequence
  Synthetic polypeptide-alpha-FOLR1 h22-4 sdAb sdCDR2
IISRGGSTNYADSVKG

SEQ ID NO: 32 - protein, 9 aa, Artificial Sequence
  Synthetic polypeptide-alpha-FOLR1 h22-4 sdAb sdCDR3
NTATWGRVF

SEQ ID NO: 33 - protein, 124 aa, Artificial Sequence
  Synthetic polypeptide-alpha-B7H3 hF7 sdAb
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSS

SEQ ID NO: 34 - protein, 10 aa, Artificial Sequence
  Synthetic polypeptide-alpha-B7H3 hF7 sdAb sdCDR1
RRTFHTYHMG

SEQ ID NO: 35 - protein, 17 aa, Artificial Sequence
  Synthetic polypeptide-alpha-B7H3 hF7 sdAb sdCDR2
VINWSGGSTVYADSVKG

SEQ ID NO: 36 - protein, 15 aa, Artificial Sequence
  Synthetic polypeptide-alpha-B7H3 hF7 sdAb sdCDR3
GGATTQRATEASYDY

SEQ ID NO: 37 - protein, 122 aa, Artificial Sequence
  Synthetic polypeptide-alpha-B7H3 hF12 sdAb
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSS

SEQ ID NO: 38 - protein, 10 aa, Artificial Sequence
  Synthetic polypeptide-alpha-B7H3 hF12 sdAb sdCDR1
PRTFSTYSMA

SEQ ID NO: 39 - protein, 17 aa, Artificial Sequence
  Synthetic polypeptide-alpha-B7H3 hF12 sdAb sdCDR2
AINWSGGNTSYADSVKG

SEQ ID NO: 40 - protein, 13 aa, Artificial Sequence
  Synthetic polypeptide-alpha-B7H3 hF12 sdAb sdCDR3
GGVLAHHNYEYDY

SEQ ID NO: 41 - protein, 122 aa, Artificial Sequence
  Synthetic polypeptide-alpha-B7H3 hF12 (N57Q) sdAb
QVQLQESGGGLVQPGGSLRLSCEASPRTFSTYSMAWFRQAPGKERSFVAAINWSGGQTSYADSVKGRFTISRDNSKNTLYLQMNSLRAEDTAVYYCAAGGVLAHHNYEYDYWGQGTLVTVSS

SEQ ID NO: 42 - protein, 10 aa, Artificial Sequence
  Synthetic polypeptide-alpha-B7H3 hF12 (N57Q) sdAb sdCDR1
PRTFSTYSMA

SEQ ID NO: 43 - protein, 17 aa, Artificial Sequence
  Synthetic polypeptide-alpha-B7H3 hF12 (N57Q) sdAb sdCDR2
AINWSGGQTSYADSVKG

SEQ ID NO: 44 - protein, 13 aa, Artificial Sequence
  Synthetic polypeptide-alpha-B7H3 hF12 (N57Q) sdAb sdCDR3
GGVLAHHNYEYDY

SEQ ID NO: 45 - protein, 122 aa, Artificial Sequence
  Synthetic polypeptide-alpha-B7H3 hF12 (N57E) sdAb
QVQLQESGGGLVQPGGSLRLSCEASPRTFSTYSMAWFRQAPGKERSFVAAINWSGGETSYADSVKGRFTISRDNSKNTLYLQMNSLRAEDTAVYYCAAGGVLAHHNYEYDYWGQGTLVTVSS

SEQ ID NO: 46 - protein, 10 aa, Artificial Sequence
  Synthetic polypeptide-alpha-B7H3 hF12 (N57E) sdAb sdCDR1
PRTFSTYSMA

SEQ ID NO: 47 - protein, 17 aa, Artificial Sequence
  Synthetic polypeptide-alpha-B7H3 hF12 (N57E) sdAb sdCDR2
AINWSGGETSYADSVKG

SEQ ID NO: 48 - protein, 13 aa, Artificial Sequence
  Synthetic polypeptide-alpha-B7H3 hF12 (N57E) sdAb sdCDR3
GGVLAHHNYEYDY

SEQ ID NO: 49 - protein, 122 aa, Artificial Sequence
  Synthetic polypeptide-alpha-B7H3 hF12 (N57D) sdAb
QVQLQESGGGLVQPGGSLRLSCEASPRTFSTYSMAWFRQAPGKERSFVAAINWSGGDTSYADSVKGRFTISRDNSKNTLYLQMNSLRAEDTAVYYCAAGGVLAHHNYEYDYWGQGTLVTVSS

SEQ ID NO: 50 - protein, 10 aa, Artificial Sequence
  Synthetic polypeptide-alpha-B7H3 hF12 (N57D) sdAb sdCDR1
PRTFSTYSMA

SEQ ID NO: 51 - protein, 17 aa, Artificial Sequence
  Synthetic polypeptide-alpha-B7H3 hF12 (N57D) sdAb sdCDR2
AINWSGGDTSYADSVKG

SEQ ID NO: 52 - protein, 13 aa, Artificial Sequence
  Synthetic polypeptide-alpha-B7H3 hF12 (N57D) sdAb sdCDR3
GGVLAHHNYEYDY

SEQ ID NO: 53 - protein, 122 aa, Artificial Sequence
  Synthetic polypeptide-alpha-B7H3 hF12 (S59A) sdAb
QVQLQESGGGLVQPGGSLRLSCEASPRTFSTYSMAWFRQAPGKERSFVAAINWSGGNTAYADSVKGRFTISRDNSKNTLYLQMNSLRAEDTAVYYCAAGGVLAHHNYEYDYWGQGTLVTVSS

SEQ ID NO: 54 - protein, 10 aa, Artificial Sequence
  Synthetic polypeptide-alpha-B7H3 hF12 (S59A) sdAb sdCDR1
PRTFSTYSMA

SEQ ID NO: 55 - protein, 17 aa, Artificial Sequence
  Synthetic polypeptide-alpha-B7H3 hF12 (S59A) sdAb sdCDR2
AINWSGGNTAYADSVKG

SEQ ID NO: 56 - protein, 13 aa, Artificial Sequence
  Synthetic polypeptide-alpha-B7H3 hF12 (S59A) sdAb sdCDR3
GGVLAHHNYEYDY

SEQ ID NO: 57 - protein, 122 aa, Artificial Sequence
  Synthetic polypeptide-alpha-B7H3 hF12 (S59Y) sdAb
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSS

SEQ ID NO: 58 - protein, 10 aa, Artificial Sequence
  Synthetic polypeptide-alpha-B7H3 hF12 (S59Y) sdAb sdCDR1
PRTFSTYSMA

SEQ ID NO: 59 - protein, 17 aa, Artificial Sequence
  Synthetic polypeptide-alpha-B7H3 hF12 (S59Y) sdAb sdCDR2
AINWSGGNTYYADSVKG

SEQ ID NO: 60 - protein, 13 aa, Artificial Sequence
  Synthetic polypeptide-alpha-B7H3 hF12 (S59Y) sdAb sdCDR3
GGVLAHHNYEYDY

SEQ ID NO: 61 - protein, 122 aa, Artificial Sequence
  Synthetic polypeptide-alpha-EpCAM h13 sdAb
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 62 - protein, 12 aa, Artificial Sequence
  Synthetic polypeptide-alpha-EpCAM h13 sdAb sdCDR1
GTGSIFSINLMG

SEQ ID NO: 63 - protein, 16 aa, Artificial Sequence
  Synthetic polypeptide-alpha-EpCAM h13 sdAb sdCDR2
RITSGDSTVYADSVKG

SEQ ID NO: 64 - protein, 12 aa, Artificial Sequence
  Synthetic polypeptide-alpha-EpCAM h13 sdAb sdCDR3
LLRSSPGATTPY

SEQ ID NO: 65 - protein, 114 aa, Artificial Sequence
  Synthetic polypeptide-alpha-EpCAM h23 sdAb
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 66 - protein, 10 aa, Artificial Sequence
  Synthetic polypeptide-alpha-EpCAM h23 sdAb sdCDR1
GSFSALWAMR

SEQ ID NO: 67 - protein, 15 aa, Artificial Sequence
  Synthetic polypeptide-alpha-EpCAM h23 sdAb sdCDR2
SSRGGTTSYADSVKG

SEQ ID NO: 68 - protein, 7 aa, Artificial Sequence
  Synthetic polypeptide-alpha-EpCAM h23 sdAb sdCDR3
IDGHLAY

SEQ ID NO: 69 - protein, 126 aa, Artificial Sequence
  Synthetic polypeptide-acEpCAM hVIB665 sdAb
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSS

SEQ ID NO: 70 - protein, 10 aa, Artificial Sequence
  Synthetic polypeptide-acEpCAM hVIB665 sdAb sdCDR1
GRTFSDYDMG

SEQ ID NO: 71 - protein, 17 aa, Artificial Sequence
  Synthetic polypeptide-acEpCAM hVIB665 sdAb sdCDR2
AISWSGGHTNYADSVKG

SEQ ID NO: 72 - protein, 17 aa, Artificial Sequence
  Synthetic polypeptide-acEpCAM hVIB665 sdAb sdCDR3
DLRFTGGDTTTPETYDY

SEQ ID NO: 73 - protein, 126 aa, Artificial Sequence
  Synthetic polypeptide-acEpCAM hVIB666 sdAb
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSS

SEQ ID NO: 74 - protein, 10 aa, Artificial Sequence
  Synthetic polypeptide-acEpCAM hVIB666 sdAb sdCDR1
GRTLDNYDMG

SEQ ID NO: 75 - protein, 17 aa, Artificial Sequence
  Synthetic polypeptide-acEpCAM hVIB666 sdAb sdCDR2
AISWSGGSTDYAYSVTG

SEQ ID NO: 76 - protein, 17 aa, Artificial Sequence
  Synthetic polypeptide-acEpCAM hVIB666 sdAb sdCDR3
DLRFTGGDTMTPETYDY

SEQ ID NO: 77 - protein, 129 aa, Artificial Sequence
  Synthetic polypeptide-aTrop2 hVIB557 sdAb
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSS

SEQ ID NO: 78 - protein, 10 aa, Artificial Sequence
  Synthetic polypeptide-aTrop2 hVIB557 sdAb sdCDR1
GRTFSSQSMG

SEQ ID NO: 79 - protein, 17 aa, Artificial Sequence
  Synthetic polypeptide-aTrop2 hVIB557 sdAb sdCDR2
AISWTGANPTYADSVKG

SEQ ID NO: 80 - protein, 20 aa, Artificial Sequence
  Synthetic polypeptide-aTrop2 hVIB557 sdAb sdCDR3
DTSGGSYYYERATAETSYDY

SEQ ID NO: 81 - protein, 128 aa, Artificial Sequence
  Synthetic polypeptide-aTrop2 hVIB565 sdAb
QVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSS

SEQ ID NO: 82 - protein, 10 aa, Artificial Sequence
  Synthetic polypeptide-aTrop2 hVIB565 sdAb sdCDR1
GFTFDYYAIG

SEQ ID NO: 83 - protein, 17 aa, Artificial Sequence
  Synthetic polypeptide-aTrop2 hVIB565 sdAb sdCDR2
CISSSHGSTYYADSVKG

SEQ ID NO: 84 - protein, 19 aa, Artificial Sequence
  Synthetic polypeptide-aTrop2 hVIB565 sdAb sdCDR3
AGDGGDYHCSGLVDYGMDY

SEQ ID NO: 85 - protein, 123 aa, Artificial Sequence
  Synthetic polypeptide-aTrop2 hVIB575 sdAb
QVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSS

SEQ ID NO: 86 - protein, 10 aa, Artificial Sequence
  Synthetic polypeptide-aTrop2 hVIB575 sdAb sdCDR1
GRTVGRTAMG

SEQ ID NO: 87 - protein, 17 aa, Artificial Sequence
  Synthetic polypeptide-aTrop2 hVIB575 sdAb sdCDR2
TISWAGGTTYYADFVKG

SEQ ID NO: 88 - protein, 14 aa, Artificial Sequence
  Synthetic polypeptide-aTrop2 hVIB575 sdAb sdCDR3
SEPYSDYDPSGMVY

SEQ ID NO: 89 - protein, 123 aa, Artificial Sequence
  Synthetic polypeptide-aTrop2 hVIB578 sdAb
QVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSS

SEQ ID NO: 90 - protein, 10 aa, Artificial Sequence
  Synthetic polypeptide-aTrop2 hVIB578 sdAb sdCDR1
GRTFGRAAMG

SEQ ID NO: 91 - protein, 17 aa, Artificial Sequence
  Synthetic polypeptide-aTrop2 hVIB578 sdAb sdCDR2
TISWSGSNTYYADFVKG

SEQ ID NO: 92 - protein, 14 aa, Artificial Sequence
  Synthetic polypeptide-aTrop2 hVIB578 sdAb sdCDR3
SEPYSDYDPSGMVY

SEQ ID NO: 93 - protein, 125 aa, Artificial Sequence
  Synthetic polypeptide-aTrop2 hVIB609 sdAb
QVQLLESGGGLVQPGGSLRLSCALSGLTFNTYPMAWFRQPPGQEREFVADMSWSGTNTYYADSVKGRFTISRDNSKNTLYLQMNSLRAEDTAVYYCAAGWPYSGTGRSTTDYTYWGQGTLVTVSS

SEQ ID NO: 94 - protein, 10 aa, Artificial Sequence
  Synthetic polypeptide-aTrop2 hVIB609 sdAb sdCDR1
GLTFNTYPMA

SEQ ID NO: 95 - protein, 17 aa, Artificial Sequence
  Synthetic polypeptide-aTrop2 hVIB609 sdAb sdCDR2
DMSWSGTNTYYADSVKG

SEQ ID NO: 96 - protein, 16 aa, Artificial Sequence
  Synthetic polypeptide-aTrop2 hVIB609 sdAb sdCDR3
GWPYSGTGRSTTDYTY

SEQ ID NO: 97 - protein, 126 aa, Artificial Sequence-aTrop2 hVIB619 sdAb
  Synthetic polypeptide-aTrop2 hVIB619 sdAb
QVQLLESGGGLVQPGGSLRLSCAASGRSFSRYGMGWLRQAPGKERELVASISWSGHSTYYADSVKGRFTISRDNSKNTLYLQMNSLRAEDTAVYYCAAESLPYESGSPRLTDFASWGQGTLVTVSS

SEQ ID NO: 98 - protein, 10 aa, Artificial Sequence
  Synthetic polypeptide-aTrop2 hVIB619 sdAb sdCDR1
GRSFSRYGMG

SEQ ID NO: 99 - protein, 17 aa, Artificial Sequence
  Synthetic polypeptide-aTrop2 hVIB619 sdAb sdCDR2
SISWSGHSTYYADSVKG

SEQ ID NO: 100 - protein, 17 aa, Artificial Sequence
  Synthetic polypeptide-aTrop2 hVIB619 sdAb sdCDR3
ESLPYESGSPRLTDFAS

SEQ ID NO: 101 - protein, 119 aa, Artificial Sequence
  Synthetic polypeptide-aCA9 hVIB456 sdAb
QVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSS

SEQ ID NO: 102 - protein, 10 aa, Artificial Sequence
  Synthetic polypeptide-aCA9 hVIB456 sdAb  sdCDR1
GSALIINAMG

SEQ ID NO: 103 - protein, 16 aa, Artificial Sequence
  Synthetic polypeptide-aCA9 hVIB456 sdAb  sdCDR2
TVTRSGRTNYADSVKG

SEQ ID NO: 104 - protein, 11 aa, Artificial Sequence
  Synthetic polypeptide-aCA9 hVIB456 sdAb  sdCDR3
ALWIADGEYDY

SEQ ID NO: 105 - protein, 116 aa, Artificial Sequence
  Synthetic polypeptide-aCA9 hVIB476 sdAb
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSS

SEQ ID NO: 106 - protein, 10 aa, Artificial Sequence
  Synthetic polypeptide-aCA9 hVIB476 sdAb  sdCDR1
GNIFIINVMG

SEQ ID NO: 107 - protein, 16 aa, Artificial Sequence
  Synthetic polypeptide-aCA9 hVIB476 sdAb  sdCDR2
TITNGGRTHYADSVKG

SEQ ID NO: 108 - protein, 8 aa, Artificial Sequence
  Synthetic polypeptide-aCA9 hVIB476 sdAb  sdCDR3
NHIELGDY

SEQ ID NO: 109 - protein, 115 aa, Artificial Sequence
  Synthetic polypeptide-aCA9 hVIB407 sdAb
QVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSS

SEQ ID NO: 110 - protein, 10 aa, Artificial Sequence
  Synthetic polypeptide-aCA9 hVIB407 sdAb  sdCDR1
GIIFSVYDMG

SEQ ID NO: 111 - protein, 16 aa, Artificial Sequence
  Synthetic polypeptide-aCA9 hVIB407 sdAb  sdCDR2
RITAGGGTYLTDSVKG

SEQ ID NO: 112 - protein, 7 aa, Artificial Sequence
  Synthetic polypeptide-aCA9 hVIB407 sdAb  sdCDR3
AWIGDDY

SEQ ID NO: 113 - protein, 115 aa, Artificial Sequence
  Synthetic polypeptide-aCA9 hVIB445 sdAb
QVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSS

SEQ ID NO: 114 - protein, 10 aa, Artificial Sequence
  Synthetic polypeptide-aCA9 hVIB445 sdAb  sdCDR1
GITFNLHAMR

SEQ ID NO: 115 - protein, 16 aa, Artificial Sequence
  Synthetic polypeptide-aCA9 hVIB445 sdAb  sdCDR2
YISARDWTNYADSVKG

SEQ ID NO: 116 - protein, 7 aa, Artificial Sequence
  Synthetic polypeptide-aCA9 hVIB445 sdAb  sdCDR3
DLVGEDY

SEQ ID NO: 117 - protein, 585 aa, Artificial Sequence
  Synthetic polypeptide-HSA sequence
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 118 - protein, 109 aa, Artificial Sequence
  Synthetic polypeptide-alpha-CD3 VL
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 119 - protein, 14 aa, Artificial Sequence
  Synthetic polypeptide-alpha-CD3 VL aVLCDR1
ASSTGAVTSGNYPN

SEQ ID NO: 120 - protein, 7 aa, Artificial Sequence
  Synthetic polypeptide-alpha-CD3 VL aVLsdCDR2
GTKFLVP

SEQ ID NO: 121 - protein, 9 aa, Artificial Sequence
  Synthetic polypeptide-alpha-CD3 VL aVLsdCDR3
TLWYSNRWV

SEQ ID NO: 122 - protein, 110 aa, Artificial Sequence
  Synthetic polypeptide-alpha-CD3 VLi
QTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVL

SEQ ID NO: 123 - protein, 14 aa, Artificial Sequence
  Synthetic polypeptide-alpha-CD3 VLi iVLCDR1
GSSTGAVTSGNYPN

SEQ ID NO: 124 - protein, 8 aa, Artificial Sequence
  Synthetic polypeptide-alpha-CD3 VLi iVLsdCDR2
DYKDDDDK

SEQ ID NO: 125 - protein, 9 aa, Artificial Sequence
  Synthetic polypeptide-alpha-CD3 VLi iVLsdCDR3
VLWYSNRWV

SEQ ID NO: 126 - protein, 109 aa, Artificial Sequence
  Synthetic polypeptide-alpha-CD3 VLi2
QTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVL

SEQ ID NO: 127 - protein, 14 aa, Artificial Sequence
  Synthetic polypeptide-alpha-CD3 VLi2 iVLCDR1
GSSTGAVTSGNYPN

SEQ ID NO: 128 - protein, 7 aa, Artificial Sequence
  Synthetic polypeptide-alpha-CD3 VLi2 iVLsdCDR2
GTKDDAP

SEQ ID NO: 129 - protein, 9 aa, Artificial Sequence
  Synthetic polypeptide-alpha-CD3 VLi2 iVLsdCDR3
VLWYSNRWV

SEQ ID NO: 130 - protein, 109 aa, Artificial Sequence
  Synthetic polypeptide-alpha-CD3 VLiGL
QTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVL

SEQ ID NO: 131 - protein, 14 aa, Artificial Sequence
  Synthetic polypeptide-alpha-CD3 VLiGL aVLiGLCDR1
GSSTGAVTSGHYPN

SEQ ID NO: 132 - protein, 7 aa, Artificial Sequence
  Synthetic polypeptide-alpha-CD3 VLiGL aVLiGLCDR2
GTSNKHS

SEQ ID NO: 133 - protein, 9 aa, Artificial Sequence
  Synthetic polypeptide-alpha-CD3 VLiGL aVLiGLCDR3
VLWGSRRWV

SEQ ID NO: 134 - protein, 125 aa, Artificial Sequence
  Synthetic polypeptide-alpha-CD3 VH
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 135 - protein, 10 aa, Artificial Sequence
  Synthetic polypeptide-alpha-CD3 VH aVHCDR1
GFTFNKYAIN

SEQ ID NO: 136 - protein, 19 aa, Artificial Sequence
  Synthetic polypeptide-alpha-CD3 VH aVHsdCDR2
RIRSKYNNYATYYADQVKD

SEQ ID NO: 137 - protein, 14 aa, Artificial Sequence
  Synthetic polypeptide-alpha-CD3 VH aVHsdCDR3
HANFGNSYISYWAY

SEQ ID NO: 138 - protein, 126 aa, Artificial Sequence
  Synthetic polypeptide-alpha-CD3 VHi
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSS

SEQ ID NO: 139 - protein, 10 aa, Artificial Sequence
  Synthetic polypeptide-alpha-CD3 VHi iVHsdCDR1
GFTFNKYAMN

SEQ ID NO: 140 - protein, 20 aa, Artificial Sequence
  Synthetic polypeptide-alpha-CD3 VHi iVHsdCDR2
RIRSKYDYKDDDDKADSVKD

SEQ ID NO: 141 - protein, 14 aa, Artificial Sequence
  Synthetic polypeptide-alpha-CD3 VHi iVHsdCDR3
HGNFGNSYISYWAY

SEQ ID NO: 142 - protein, 125 aa, Artificial Sequence
  Synthetic polypeptide-alpha-CD3 VHi2
EVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSS

SEQ ID NO: 143 - protein, 10 aa, Artificial Sequence
  Synthetic polypeptide-alpha-CD3 VHi2 iVHsdCDR1
GFTFNKHAMN

SEQ ID NO: 144 - protein, 19 aa, Artificial Sequence
  Synthetic polypeptide-alpha-CD3 VHi2 iVHsdCDR2
RIRSKYNNYATAYADSVKD

SEQ ID NO: 145 - protein, 14 aa, Artificial Sequence
  Synthetic polypeptide-alpha-CD3 VHi2 iVHsdCDR3
HGNFGNSYISYWAY

SEQ ID NO: 146 - protein, 125 aa, Artificial Sequence
  Synthetic polypeptide-alpha-CD3 VHiGL4
EVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSS

SEQ ID NO: 147 - protein, 10 aa, Artificial Sequence
  Synthetic polypeptide-alpha-CD3 VHiGL4 aVHiGL4CDR1
GFTFSGYAMN

SEQ ID NO: 148 - protein, 19 aa, Artificial Sequence
  Synthetic polypeptide-alpha-CD3 VHiGL4 aVHiGL4CDR2
RIRSKANSYATEYAASVKD

SEQ ID NO: 149 - protein, 14 aa, Artificial Sequence
  Synthetic polypeptide-alpha-CD3 VHiGL4 aVHiGL4CDR3
HGNAGNSAISYWAY

SEQ ID NO: 150 - protein, 15 aa, Artificial Sequence
  Synthetic polypeptide-Normal/Non-cleavable linker
GGGGSGGGGSGGGGS

SEQ ID NO: 151 - protein, 8 aa, Artificial Sequence
  Synthetic polypeptide-Constrained linker
GGGSGGGS

SEQ ID NO: 152 - protein, 8 aa, Artificial Sequence
  Synthetic polypeptide-MMP 2/9
GPAGMKGL

SEQ ID NO: 153 - protein, 15 aa, Artificial Sequence
  Synthetic polypeptide-MMP 2/9
SGGPGPAGMKGLPGS

SEQ ID NO: 154 - protein, 17 aa, Artificial Sequence
  Synthetic polypeptide-MMP 2/9
SGGGPGPAGMKGLPGGS

SEQ ID NO: 155 - protein, 16 aa, Artificial Sequence
  Synthetic polypeptide-Meprin A/B
GGGGKKLADEPEGGGS

SEQ ID NO: 156 - protein, 15 aa, Artificial Sequence
  Synthetic polypeptide-Meprin A/B
SGGGKKLADEPEGGS

SEQ ID NO: 157 - protein, 8 aa, Artificial Sequence
  Synthetic polypeptide-Meprin A/B
KKLADEPE

SEQ ID NO: 158 - protein, 13 aa, Artificial Sequence
  Synthetic polypeptide-Meprin A/B (Variant, high efficiency)
GGGKFLADEPEGG

SEQ ID NO: 159 - protein, 15 aa, Artificial Sequence
  Synthetic polypeptide-Cathepsin S, K, L
GGGARLQSAAPGGGS

SEQ ID NO: 160 - protein, 16 aa, Artificial Sequence
  Synthetic polypeptide-Cathepsin S, K, L
SGGGARLQSAAPGGGS

SEQ ID NO: 161 - protein, 8 aa, Artificial Sequence
  Synthetic polypeptide-Cathepsin S, K, L
ARLQSAAP

SEQ ID NO: 162 - protein, 18 aa, Artificial Sequence
  Synthetic polypeptide-Meprin/Granzyme B
SGGGGVYADSLEDGGGGS

SEQ ID NO: 163 - protein, 10 aa, Artificial Sequence
  Synthetic polypeptide-Meprin/Granzyme B
GVYADSLEDG

SEQ ID NO: 164 - protein, 16 aa, Artificial Sequence
  Synthetic polypeptide-Matriptase/uPA (MS)
GGGSLSGRSDNHGGGS

SEQ ID NO: 165 - protein, 10 aa, Artificial Sequence
  Synthetic polypeptide-Matriptase/uPA (MS)
GLSGRSDNHG

SEQ ID NO: 166 - protein, 17 aa, Artificial Sequence
  Synthetic polypeptide-Matriptase (MV)
SGGGSFTRQARVVGGGS

SEQ ID NO: 167 - protein, 9 aa, Artificial Sequence
  Synthetic polypeptide-Matriptase (MV)
SFTRQARVV

SEQ ID NO: 168 - protein, 14 aa, Artificial Sequence
  Synthetic polypeptide-CathepsinS/MMP9/Meprin A
ARLQSAAPAGLKGA

SEQ ID NO: 169 - protein, 16 aa, Artificial Sequence
  Synthetic polypeptide-CathepsinS/MMP9/Meprin A
GARLQSAAPAGLKGAG

SEQ ID NO: 170 - protein, 13 aa, Artificial Sequence
  Synthetic polypeptide-MMP9 (Variant, high efficiency)
GGPGPAGMHGLPG

SEQ ID NO: 171 - protein, 17 aa, Artificial Sequence
  Synthetic polypeptide-MMP9 (Variant, high efficiency)
GSGGPGPAGMHGLPGGS

SEQ ID NO: 172 - protein, 13 aa, Artificial Sequence
  Synthetic polypeptide-MMP9 (Variant, low efficiency)
GGPGPAGMEGLPG

SEQ ID NO: 173 - protein, 15 aa, Artificial Sequence
  Synthetic polypeptide-MMP9-15 (K-E)
SGGPGPAGMEGLPGS

SEQ ID NO: 174 - protein, 15 aa, Artificial Sequence
  Synthetic polypeptide-MMP9-15 (M-P)
SGGPGPAGPKGLPGS

SEQ ID NO: 175 - protein, 16 aa, Artificial Sequence
  Synthetic polypeptide-Thrombin 1
GGGGLVPRGSLGGGGS

SEQ ID NO: 176 - protein, 15 aa, Artificial Sequence
  Synthetic polypeptide-Thrombin 2
SSGGGMPRSFRGGGS

SEQ ID NO: 177 - protein, 16 aa, Artificial Sequence
  Synthetic polypeptide-Enterokinase/Flag
GGGGDYKDDDDKGGGS

SEQ ID NO: 178 - protein, 16 aa, Artificial Sequence
  Synthetic polypeptide-KLK7-6
SGGGQNPYSAGRGGGS

SEQ ID NO: 179 - protein, 16 aa, Artificial Sequence
  Synthetic polypeptide-KLK7-13
SGGGQNPYSAGGGSGG

SEQ ID NO: 180 - protein, 16 aa, Artificial Sequence
  Synthetic polypeptide-KLK7-11
SGGGRNVYSAGGGSGG

SEQ ID NO: 181 - protein, 16 aa, Artificial Sequence
  Synthetic polypeptide-KLK7-10
SGGGQNTWSAGKGGGS

SEQ ID NO: 182 - protein, 16 aa, Artificial Sequence
  Synthetic polypeptide-uPA
GGGSHTGRSAYFGGGS

SEQ ID NO: 183 - protein, 15 aa, Artificial Sequence
  Synthetic polypeptide-MMP9-2
SGGPGPAGLKGAPGS

SEQ ID NO: 184 - protein, 8 aa, Artificial Sequence
  Synthetic polypeptide-MMP7
KRALGLPG

SEQ ID NO: 185 - protein, 40 aa, Artificial Sequence
  Synthetic polypeptide-MMP7
DEDEDEDEDEDEDEDERPLALWRSDRDRDRDRDRDRDRDR

SEQ ID NO: 186 - protein, 5 aa, Artificial Sequence
  Synthetic polypeptide-MMP9
  SITE (1)..(5): Variant residues given in the sequence have no preference with respect to those in the annotations for variant positions
  VARIANT (3)..(3): replace=“T”
  VARIANT (4)..(4): replace=“I”
  VARIANT (5)..(5): replace=“T”
PRSLS

SEQ ID NO: 187 - protein, 5 aa, Artificial Sequence
  Synthetic polypeptide-MMP9
LEATA

SEQ ID NO: 188 - protein, 10 aa, Artificial Sequence
  Synthetic polypeptide-MMP11
GGAANLVRGG

SEQ ID NO: 189 - protein, 10 aa, Artificial Sequence
  Synthetic polypeptide-MMP14
SGRIGFLRTA

SEQ ID NO: 190 - protein, 6 aa, Artificial Sequence
  Synthetic polypeptide-MMP
PLGLAG

SEQ ID NO: 191 - protein, 6 aa, Artificial Sequence
  Synthetic polypeptide-MMP
  misc_feature (6)..(6): Xaa can be any naturally occurring amino acid
PLGLAX

SEQ ID NO: 192 - protein, 6 aa, Artificial Sequence
  Synthetic polypeptide-MMP
  MOD_RES (4)..(4): Xaa is Cys(Me)
PLGXAG

SEQ ID NO: 193 - protein, 8 aa, Artificial Sequence
  Synthetic polypeptide-MMP
ESPAYYTA

SEQ ID NO: 194 - protein, 6 aa, Artificial Sequence
  Synthetic polypeptide-MMP
RLQLKL

SEQ ID NO: 195 - protein, 7 aa, Artificial Sequence
  Synthetic polypeptide-MMP
RLQLKAC

SEQ ID NO: 196 - protein, 7 aa, Artificial Sequence
  Synthetic polypeptide-MMP2, MMP9, MMP14
  MOD_RES (3)..(3): Xaa is Cit
  MOD_RES (5)..(5): Xaa is Hof
EPXGXYL

SEQ ID NO: 197 - protein, 5 aa, Artificial Sequence
  Synthetic polypeptide-Urokinase plasminogen activator (upa)
SGRSA

SEQ ID NO: 198 - protein, 4 aa, Artificial Sequence
  Synthetic polypeptide-Urokinase plasminogen activator (upa)
DAFK

SEQ ID NO: 199 - protein, 5 aa, Artificial Sequence
  Synthetic polypeptide-Urokinase plasminogen activator (upa)
GGGRR

SEQ ID NO: 200 - protein, 4 aa, Artificial Sequence
  Synthetic polypeptide-Lysosomal enzyme
GFLG

SEQ ID NO: 201 - protein, 4 aa, Artificial Sequence
  Synthetic polypeptide-Lysosomal enzyme
ALAL

SEQ ID NO: 202 - protein, 2 aa, Artificial Sequence
  Synthetic polypeptide-Lysosomal enzyme
FK

SEQ ID NO: 203 - protein, 3 aa, Artificial Sequence
  Synthetic polypeptide-Cathepsin B
NLL

SEQ ID NO: 204 - protein, 5 aa, Artificial Sequence
  Synthetic polypeptide-Cathepsin D
  MOD_RES (3)..(3): Xaa is Cys(Et)
PIXFF

SEQ ID NO: 205 - protein, 8 aa, Artificial Sequence
  Synthetic polypeptide-athepsin K
GGPRGLPG

SEQ ID NO: 206 - protein, 6 aa, Artificial Sequence
  Synthetic polypeptide-Prostate specific antigen
HSSKLQ

SEQ ID NO: 207 - protein, 7 aa, Artificial Sequence
  Synthetic polypeptide-rostate specific antigen
HSSKLQL

SEQ ID NO: 208 - protein, 9 aa, Artificial Sequence
  Synthetic polypeptide-Prostate specific antigen
HSSKLQEDA

SEQ ID NO: 209 - protein, 10 aa, Artificial Sequence
  Synthetic polypeptide-Herpes simplex virus protease
LVLASSSFGY

SEQ ID NO: 210 - protein, 10 aa, Artificial Sequence
  Synthetic polypeptide-HIV protease
GVSQNYPIVG

SEQ ID NO: 211 - protein, 10 aa, Artificial Sequence
  Synthetic polypeptide-CMV protease
GVVQASCRLA

SEQ ID NO: 212 - protein, 4 aa, Artificial Sequence
  Synthetic polypeptide-Thrombin
  MOD_RES (2)..(2): Xaa is Pip
FXRS

SEQ ID NO: 213 - protein, 6 aa, Artificial Sequence
  Synthetic polypeptide-Thrombin
DPRSFL

SEQ ID NO: 214 - protein, 6 aa, Artificial Sequence
  Synthetic polypeptide-Thrombin
PPRSFL

SEQ ID NO: 215 - protein, 4 aa, Artificial Sequence
  Synthetic polypeptide-Caspase-3
DEVD

SEQ ID NO: 216 - protein, 5 aa, Artificial Sequence
  Synthetic polypeptide-Caspase-3
DEVDP

SEQ ID NO: 217 - protein, 8 aa, Artificial Sequence
  Synthetic polypeptide-Caspase-3
KGSGDVEG

SEQ ID NO: 218 - protein, 6 aa, Artificial Sequence
  Synthetic polypeptide-Interleukin 1-beta converting enzyme
GWEHDG

SEQ ID NO: 219 - protein, 7 aa, Artificial Sequence
  Synthetic polypeptide-Enterokinase
EDDDDKA

SEQ ID NO: 220 - protein, 9 aa, Artificial Sequence
  Synthetic polypeptide-Fap
KQEQNPGST

SEQ ID NO: 221 - protein, 6 aa, Artificial Sequence
  Synthetic polypeptide-Kallikrein 2
GKAFRR

SEQ ID NO: 222 - protein, 4 aa, Artificial Sequence
  Synthetic polypeptide-Plasmin
DAFK

SEQ ID NO: 223 - protein, 4 aa, Artificial Sequence
  Synthetic polypeptide-Plasmin
DVLK

SEQ ID NO: 224 - protein, 4 aa, Artificial Sequence
  Synthetic polypeptide-Plasmin
DAFK

SEQ ID NO: 225 - protein, 7 aa, Artificial Sequence
  Synthetic polypeptide-Top
ALLLALL

SEQ ID NO: 226 - protein, 1359 aa, Artificial Sequence
  Synthetic polypeptide-Pro817 - Pro186/HSA Fusion (D12)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 227 - protein, 1359 aa, Artificial Sequence
  Synthetic polypeptide-Pro963 - Pro743/HSA fusion (hD12a)
EVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 228 - protein, 1361 aa, Artificial Sequence
  Synthetic polypeptide-Pro964 - Pro753/ HSA fusion (hD12d/MMP9v linker)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 229 - protein, 1359 aa, Artificial Sequence
  Synthetic polypeptide-Pro965 - Pro742/HSA fusion (hD12d/MMP9 linker)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 230 - protein, 1359 aa, Artificial Sequence
  Synthetic polypeptide-Pro966 - Pro502/HSA fusion (hF7)
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 231 - protein, 1355 aa, Artificial Sequence
  Synthetic polypeptide-Pro967 - Pro740/HSA fusion (hF12 S59Y)
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 232 - protein, 895 aa, Artificial Sequence
  Synthetic polypeptide-Pro186
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 233 - protein, 514 aa, Artificial Sequence
  Synthetic polypeptide-Pro201
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAG

SEQ ID NO: 234 - protein, 895 aa, Artificial Sequence
  Synthetic polypeptide-Pro214
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 235 - protein, 1357 aa, Artificial Sequence
  Synthetic polypeptide-Pro1109-HSA
QVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 236 - protein, 1365 aa, Artificial Sequence
  Synthetic polypeptide-Pro1111-HSA
QVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 237 - protein, 1347 aa, Artificial Sequence
  Synthetic polypeptide-Pro1117-HSA
QVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 238 - protein, 1349 aa, Artificial Sequence
  Synthetic polypeptide-Pro1118-HSA
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 239 - protein, 1341 aa, Artificial Sequence
  Synthetic polypeptide-Pro518-HSA
QVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 240 - protein, 1341 aa, Artificial Sequence
  Synthetic polypeptide-Pro519-HSA
QVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 241 - protein, 1349 aa, Artificial Sequence
  Synthetic polypeptide-Pro516-HSA
QVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 242 - protein, 1343 aa, Artificial Sequence
  Synthetic polypeptide-Pro517-HSA
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 243 - protein, 1359 aa, Artificial Sequence
  Synthetic polypeptide-Pro974
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 244 - protein, 40 aa, Artificial Sequence
  Synthetic polypeptide
  SITE (1)..(40): This sequence may encompass 1-10 ′Gly Gly Gly Ser′ repeating units
GGGSGGGSGGGSGGGSGGGSGGGSGGGSGGGSGGGSGGGS

SEQ ID NO: 245 - protein, 40 aa, Artificial Sequence
  Synthetic polypeptide
  SITE (1)..(40): This sequence may encompass 1-10 ′Gly Gly Ser Gly′ repeating units
GGSGGGSGGGSGGGSGGGSGGGSGGGSGGGSGGGSGGGSG

SEQ ID NO: 246 - protein, 50 aa, Artificial Sequence
  Synthetic polypeptide
  SITE (1)..(50): This sequence may encompass 1-10 ′Gly Gly Ser Gly Gly′ repeating units
GGSGGGGSGGGGSGGGGSGGGGSGGGGSGGGGSGGGGSGGGGSGGGGSGG

SEQ ID NO: 247 - protein, 50 aa, Artificial Sequence
  Synthetic polypeptide
  SITE (1)..(50): This sequence may encompass 1-10 ′Gly Gly Gly Gly Ser′ repeating units
GGGGSGGGGSGGGGSGGGGSGGGGSGGGGSGGGGSGGGGSGGGGSGGGGS

SEQ ID NO: 248 - protein, 123 aa, Artificial Sequence
  Synthetic polypeptide-alpha-LyPD3 h787 sdAb
QVQLLESGGGLVQPGGSLRLSCAASGFTLDYYAIGWFRQAPGKEHEAVSCISSSDGSTYYADSVKGRFTISRDNSKNTLYLQMNSLRAEDTAVYYCATVKSCYSDGYALWGSWGQGTLVTVSS

SEQ ID NO: 249 - protein, 10 aa, Artificial Sequence
  Synthetic polypeptide-alpha-LyPD3 h787 sdAb sdCDR1
GFTLDYYAIG

SEQ ID NO: 250 - protein, 17 aa, Artificial Sequence
  Synthetic polypeptide-alpha-LyPD3 h787 sdAb sdCDR2
CISSSDGSTYYADSVKG

SEQ ID NO: 251 - protein, 14 aa, Artificial Sequence
  Synthetic polypeptide-alpha-LyPD3 h787 sdAb sdCDR3
VKSCYSDGYALWGS

SEQ ID NO: 252 - protein, 123 aa, Artificial Sequence
  Synthetic polypeptide-alpha-LyPD3 h790 sdAb
QVQLLESGGGLVQPGGSLRLSCAASGFTLDYYSIGWFRQAPGKEREGVSCISSSDGSTYYADSVKGRFTISRDNSKNTLYLQMNSLRAEDTAVYYCATVKSCYVDGYILWGSWGQGTLVTVSS

SEQ ID NO: 253 - protein, 10 aa, Artificial Sequence
  Synthetic polypeptide-alpha-LyPD3 h790 sdAb sdCDR1
GFTLDYYSIG

SEQ ID NO: 254 - protein, 17 aa, Artificial Sequence
  Synthetic polypeptide-alpha-LyPD3 h790 sdAb sdCDR2
CISSSDGSTYYADSVKG

SEQ ID NO: 255 - protein, 14 aa, Artificial Sequence
  Synthetic polypeptide-alpha-LyPD3 h790 sdAb sdCDR3
VKSCYVDGYILWGS

SEQ ID NO: 256 - protein, 121 aa, Artificial Sequence
  Synthetic polypeptide-alpha-LyPD3 H804 sdAb
QVQLLESGGGLVQPGGSLRLSCAASGFTLDYYAIGWFRQAPGKEHEGVSCISSSDGSTYYADSVKGRFTISRDNSKNTLYLQMNSLRAEDTAVYYCATVRSCASPYELGSWGQGTLVTVSS

SEQ ID NO: 257 - protein, 10 aa, Artificial Sequence
  Synthetic polypeptide-alpha-LyPD3 H804 sdAb sdCDR1
GFTLDYYAIG

SEQ ID NO: 258 - protein, 17 aa, Artificial Sequence
  Synthetic polypeptide-alpha-LyPD3 H804 sdAb sdCDR2
CISSSDGSTYYADSVKG

SEQ ID NO: 259 - protein, 12 aa, Artificial Sequence
  Synthetic polypeptide-alpha-LyPD3 H804 sdAb sdCDR3
VRSCASPYELGS

SEQ ID NO: 260 - protein, 124 aa, Artificial Sequence
  Synthetic polypeptide-alpha-LyPD h773 sdAb
QVQLVESGGGLVQPGGSLRLSCAASGRSFSSYGMGWFRQAPGKEREIVAAISWGGSAVYYANSVKGRFTISRDNSKNTLYLQMNSLRAEDTAVYYCAANRQGRALVRKEFYDYWGQGTLVTVSS

SEQ ID NO: 261 - protein, 10 aa, Artificial Sequence
  Synthetic polypeptide-alpha-LyPD h773 sdAb sdCDR1
GRSFSSYGMG

SEQ ID NO: 262 - protein, 17 aa, Artificial Sequence
  Synthetic polypeptide-alpha-LyPD h773 sdAb sdCDR2
AISWGGSAVYYANSVKG

SEQ ID NO: 263 - protein, 15 aa, Artificial Sequence
  Synthetic polypeptide-alpha-LyPD h773 sdAb sdCDR3
NRQGRALVRKEFYDY

SEQ ID NO: 264 - protein, 117 aa, Artificial Sequence
  Synthetic polypeptide-alpha-LyPD3 h840 sdAb
QVQLLESGGGKVQPGGSLRLSCVVSGDTSSIYLMGWYRQTPGKQRELVAHIRGNGRTNYADSVKGRFTISRDNSKNTLYLQMNSLRAEDTAVYYCNVRNFWGSAEYWGQGTLVTVSS

SEQ ID NO: 265 - protein, 10 aa, Artificial Sequence
  Synthetic polypeptide-alpha-LyPD3 h840 sdAb sdCDR1
GDTSSIYLMG

SEQ ID NO: 266 - protein, 16 aa, Artificial Sequence
  Synthetic polypeptide-alpha-LyPD3 h840 sdAb sdCDR2
HIRGNGRTNYADSVKG

SEQ ID NO: 267 - protein, 9 aa, Artificial Sequence
  Synthetic polypeptide-alpha-LyPD3 h840 sdAb sdCDR3
RNFWGSAEY

SEQ ID NO: 268 - protein, 124 aa, Artificial Sequence
  Synthetic polypeptide-alpha-LyPD3 h885 sdAb
QVQLLESGGGLVQPGGSLRLSCVASRGTFSRYSMGWFRQAPGKEREFVGRISWSGDMTYYADSVKGRFTISRDNSKNTLYLQMNSLRAEDTAVYYCAASTLGYVWNHPNMYGYWGHGTLVTVSS

SEQ ID NO: 269 - protein, 10 aa, Artificial Sequence
  Synthetic polypeptide-alpha-LyPD3 h885 sdAb sdCDR1
RGTFSRYSMG

SEQ ID NO: 270 - protein, 17 aa, Artificial Sequence
  Synthetic polypeptide-alpha-LyPD3 h885 sdAb sdCDR2
RISWSGDMTYYADSVKG

SEQ ID NO: 271 - protein, 15 aa, Artificial Sequence
  Synthetic polypeptide-alpha-LyPD3 h885 sdAb sdCDR3
STLGYVWNHPNMYGY

SEQ ID NO: 272 - protein, 118 aa, Artificial Sequence
  Synthetic polypeptide-alpha-HER2 1054 sdAb
QVQLQESGGGLVQAGGSLILSCAASGRTFSNYAMGWFRQAPGKEREFVAAINWSGTHTDYADSVKGRFTISRDNVKRTVYLQMNSLKPEDTATYFCSRGWSVFDPDYRGQGTQVTVSS

SEQ ID NO: 273 - protein, 10 aa, Artificial Sequence
  Synthetic polypeptide-alpha-HER2 1054 sdAb sdCDR1
GRTFSNYAMG

SEQ ID NO: 274 - protein, 17 aa, Artificial Sequence
  Synthetic polypeptide-alpha-HER2 1054 sdAb sdCDR2
AINWSGTHTDYADSVKG

SEQ ID NO: 275 - protein, 9 aa, Artificial Sequence
  Synthetic polypeptide-alpha-HER2 1054 sdAb sdCDR3
GWSVFDPDY

SEQ ID NO: 276 - protein, 118 aa, Artificial Sequence
  Synthetic polypeptide-alpha-HER2 1055 sdAb
QVQLQESGGGLVQPGGSLRLTCEVSGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 277 - protein, 10 aa, Artificial Sequence
  Synthetic polypeptide-alpha-HER2 1055 sdAb sdCDR1
GRAISSYAIS

SEQ ID NO: 278 - protein, 17 aa, Artificial Sequence
  Synthetic polypeptide-alpha-HER2 1055 sdAb sdCDR2
AINWSGTHTNYADSVKG

SEQ ID NO: 279 - protein, 10 aa, Artificial Sequence
  Synthetic polypeptide-alpha-HER2 1055 sdAb sdCDR3
GWSVFDPDYR

SEQ ID NO: 280 - protein, 118 aa, Artificial Sequence
  Synthetic polypeptide-alpha-HER2 1058 sdAb
QVQLQESGGGLVQPGGSLRLTCAASGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 281 - protein, 10 aa, Artificial Sequence
  Synthetic polypeptide-alpha-HER2 1058 sdAb sdCDR1
GRAISSYAIS

SEQ ID NO: 282 - protein, 17 aa, Artificial Sequence
  Synthetic polypeptide-alpha-HER2 1058 sdAb sdCDR2
AINWSGTHTNYADSVKG

SEQ ID NO: 283 - protein, 9 aa, Artificial Sequence
  Synthetic polypeptide-alpha-HER2 1058 sdAb sdCDR3
GWSVFDPDY

SEQ ID NO: 284 - protein, 118 aa, Artificial Sequence
  Synthetic polypeptide-alpha-HER2 1059 sdAb
QVQLQESGGGLVQPGGSLRLTCAASGRAISSYAISWFRQAPGEEREFVAAINWSGTHTDYADSVKGRFTISRDNAKNTVYLQMNSLKPEDTAVYFCSRGWSVFDPDYRGQGTQVTVSS

SEQ ID NO: 285 - protein, 10 aa, Artificial Sequence
  Synthetic polypeptide-alpha-HER2 1059 sdAb sdCDR1
GRAISSYAIS

SEQ ID NO: 286 - protein, 17 aa, Artificial Sequence
  Synthetic polypeptide-alpha-HER2 1059 sdAb sdCDR2
AINWSGTHTDYADSVKG

SEQ ID NO: 287 - protein, 9 aa, Artificial Sequence
  Synthetic polypeptide-alpha-HER2 1059 sdAb sdCDR3
GWSVFDPDY

SEQ ID NO: 288 - protein, 118 aa, Artificial Sequence
  Synthetic polypeptide-alpha-HER2 1065 sdAb
QVQLQESGGGLVQPGGSLRLTCEVSGRAISSYAISWFRQAPGEEREFVAAINWSGTHTNYADSVKGRFTISRDNVKRTVYLQMNSLKPEDTATYFCSRGWSVFDPDYRGQGTQVTVSS

SEQ ID NO: 289 - protein, 10 aa, Artificial Sequence
  Synthetic polypeptide-alpha-HER2 1065 sdAb sdCDR1
GRAISSYAIS

SEQ ID NO: 290 - protein, 17 aa, Artificial Sequence
  Synthetic polypeptide-alpha-HER2 1065 sdAb sdCDR2
AINWSGTHTNYADSVKG

SEQ ID NO: 291 - protein, 9 aa, Artificial Sequence
  Synthetic polypeptide-alpha-HER2 1065 sdAb sdCDR3
GWSVFDPDY

SEQ ID NO: 292 - protein, 118 aa, Artificial Sequence
  Synthetic polypeptide-alpha-HER2 1090 sdAb
QVQLQESGGGLVQTGGSLRLTCEVSGRAISSYAISWFRQAPGEEREFVAAINWSGTHTNYADSVKGRFTISRDNVKNTVYLQMNSLKPEDTATYFCSRGWSLFDPDYRGQGTQVTVSS

SEQ ID NO: 293 - protein, 10 aa, Artificial Sequence
  Synthetic polypeptide-alpha-HER2 1090 sdAb sdCDR1
GRAISSYAIS

SEQ ID NO: 294 - protein, 17 aa, Artificial Sequence
  Synthetic polypeptide-alpha-HER2 1090 sdAb sdCDR2
AINWSGTHTNYADSVKG

SEQ ID NO: 295 - protein, 9 aa, Artificial Sequence
  Synthetic polypeptide-alpha-HER2 1090 sdAb sdCDR3
GWSLFDPDY

SEQ ID NO: 296 - protein, 121 aa, Artificial Sequence
  Synthetic polypeptide-alpha-HER2 1091 sdAb
QVQLQESGGGLVQPGGSLRLSCAASGFTFDDYAMSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 297 - protein, 10 aa, Artificial Sequence
  Synthetic polypeptide-alpha-HER2 1091 sdAb sdCDR1
GFTFDDYAMS

SEQ ID NO: 298 - protein, 17 aa, Artificial Sequence
  Synthetic polypeptide-alpha-HER2 1091 sdAb sdCDR2
SINWSGTHTDYADSVKG

SEQ ID NO: 299 - protein, 12 aa, Artificial Sequence
  Synthetic polypeptide-alpha-HER2 1091 sdAb sdCDR3
GWRLTGGYYDSV

SEQ ID NO: 300 - protein, 121 aa, Artificial Sequence
  Synthetic polypeptide-alpha-HER2 1092 sdAb
QVQLQESGGGLVQAGASLRLSCAASGFTFDDYALSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 301 - protein, 10 aa, Artificial Sequence
  Synthetic polypeptide-alpha-HER2 1092 sdAb sdCDR1
GFTFDDYALS

SEQ ID NO: 302 - protein, 17 aa, Artificial Sequence
  Synthetic polypeptide-alpha-HER2 1092 sdAb sdCDR2
SINWSGTHTDYADSVKG

SEQ ID NO: 303 - protein, 12 aa, Artificial Sequence
  Synthetic polypeptide-alpha-HER2 1092 sdAb sdCDR3
GWRLTGGYYDSV

SEQ ID NO: 304 - protein, 121 aa, Artificial Sequence
  Synthetic polypeptide-alpha-HER2 1097 sdAb
QVQLQESGGGLVQPGGSLRLSCAASGFIFDDYAMTWVRQAPGKGLEWVSSINWSGTHTDYADSVKGRFTISRDNAKNTLYLQMNSLKSEDSAVYYCVKGWRHTDNYYAGVQGQGTQVTVSS

SEQ ID NO: 305 - protein, 10 aa, Artificial Sequence
  Synthetic polypeptide-alpha-HER2 1097 sdAb sdCDR1
GFIFDDYAMT

SEQ ID NO: 306 - protein, 17 aa, Artificial Sequence
  Synthetic polypeptide-alpha-HER2 1097 sdAb sdCDR2
SINWSGTHTDYADSVKG

SEQ ID NO: 307 - protein, 12 aa, Artificial Sequence
  Synthetic polypeptide-alpha-HER2 1097 sdAb sdCDR3
GWRHTDNYYAGV

SEQ ID NO: 308 - protein, 118 aa, Artificial Sequence
  Synthetic polypeptide-alpha-HER2 1118 sdAb
QVQLQESGGGLVQAGGSLRLSCAASGFTFDDYAMSWVRQTPGKGLEWVSSINWSGTHTNYADSVKGRFTISRDNAKKTLYLQMNSLKSEDTAVYYCARGWMVVPDSTRGQGTQVTVSS

SEQ ID NO: 309 - protein, 10 aa, Artificial Sequence
  Synthetic polypeptide-alpha-HER2 1118 sdAb sdCDR1
GFTFDDYAMS

SEQ ID NO: 310 - protein, 17 aa, Artificial Sequence
  Synthetic polypeptide-alpha-HER2 1118 sdAb sdCDR2
SINWSGTHTNYADSVKG

SEQ ID NO: 311 - protein, 9 aa, Artificial Sequence
  Synthetic polypeptide-alpha-HER2 1118 sdAb sdCDR3
GWMVVPDST

SEQ ID NO: 312 - protein, 118 aa, Artificial Sequence
  Synthetic polypeptide-alpha-HER2 1121 sdAb
QVQLQESGGGLVQAGGSLRLSCAASGFIFSDYAMTWVRQAPGKGLEWVSSINWSGTHTNYADSVKGRFTISRDNAKKTLYLQMNSLKSEDTAVYYCARGWMVVPDSTRGQGTQVTVSS

SEQ ID NO: 313 - protein, 10 aa, Artificial Sequence
  Synthetic polypeptide-alpha-HER2 1121 sdAb sdCDR1
GFIFSDYAMT

SEQ ID NO: 314 - protein, 17 aa, Artificial Sequence
  Synthetic polypeptide-alpha-HER2 1121 sdAb sdCDR2
SINWSGTHTNYADSVKG

SEQ ID NO: 315 - protein, 9 aa, Artificial Sequence
  Synthetic polypeptide-alpha-HER2 1121 sdAb sdCDR3
GWMVVPDST

SEQ ID NO: 316 - protein, 113 aa, Artificial Sequence
  Synthetic polypeptide-alpha-HER2 1134 sdAb
QVQLQESGGGLVQAGGSLRLSCAASGRIFSAAVMAWYRQPPGEQRELVASITHRGFTRYADSVKDRFTISRDNVNNTMYLQMNRLSPEDTAIYYCRLLNGDYWGQGTQVTVSS

SEQ ID NO: 317 - protein, 10 aa, Artificial Sequence
  Synthetic polypeptide-alpha-HER2 1134 sdAb sdCDR1
GRIFSAAVMA

SEQ ID NO: 318 - protein, 16 aa, Artificial Sequence
  Synthetic polypeptide-alpha-HER2 1134 sdAb sdCDR2
SITHRGFTRYADSVKD

SEQ ID NO: 319 - protein, 5 aa, Artificial Sequence
  Synthetic polypeptide-alpha-HER2 1134 sdAb sdCDR3
LNGDY

SEQ ID NO: 320 - protein, 118 aa, Artificial Sequence
  Synthetic polypeptide-alpha-HER2 1138 sdAb
QVQLQESGGGLVQPGGSLRLSCAASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 321 - protein, 10 aa, Artificial Sequence
  Synthetic polypeptide-alpha-HER2 1138 sdAb sdCDR1
GFTFDDYAMS

SEQ ID NO: 322 - protein, 17 aa, Artificial Sequence
  Synthetic polypeptide-alpha-HER2 1138 sdAb sdCDR2
GINWSGTHTRYADSVKG

SEQ ID NO: 323 - protein, 9 aa, Artificial Sequence
  Synthetic polypeptide-alpha-HER2 1138 sdAb sdCDR3
GFAADSRST

SEQ ID NO: 324 - protein, 118 aa, Artificial Sequence
  Synthetic polypeptide-alpha-HER2 1139 sdAb
QVQLQESGGGLVQPGGSLRLSCAASGFTFDDYAMSWIRQAPGKEMEWVSGINWSGTHTRYADSVKGRFTISRDNAKNTLDLQMNSLISEDTAVYYCARGFAADSRSTRGQGTQVTVSS

SEQ ID NO: 325 - protein, 10 aa, Artificial Sequence
  Synthetic polypeptide-alpha-HER2 1139 sdAb sdCDR1
GFTFDDYAMS

SEQ ID NO: 326 - protein, 17 aa, Artificial Sequence
  Synthetic polypeptide-alpha-HER2 1139 sdAb sdCDR2
GINWSGTHTRYADSVKG

SEQ ID NO: 327 - protein, 9 aa, Artificial Sequence
  Synthetic polypeptide-alpha-HER2 1139 sdAb sdCDR3
GFAADSRST

SEQ ID NO: 328 - protein, 118 aa, Artificial Sequence
  Synthetic polypeptide-alpha-HER2 1140 sdAb
QVQLQESGGGLVQPGGSLRLSCVASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 329 - protein, 10 aa, Artificial Sequence
  Synthetic polypeptide-alpha-HER2 1140 sdAb sdCDR1
GFTFDDYAMS

SEQ ID NO: 330 - protein, 17 aa, Artificial Sequence
  Synthetic polypeptide-alpha-HER2 1140 sdAb sdCDR2
GINWSGTHTRYADSVKG

SEQ ID NO: 331 - protein, 9 aa, Artificial Sequence
  Synthetic polypeptide-alpha-HER2 1140 sdAb sdCDR3
GFAADSRST

SEQ ID NO: 332 - protein, 125 aa, Artificial Sequence
  Synthetic polypeptide-alpha-HER2 1145 sdAb
QVQLQESGGGLVQAGGSLRLSCAASGRIFKNYPMGWFRQPPGKDREFVAAITSSGGTTHYRDSVKGRFTISRDNAKNTVYLQMNSLKPDDTAVYYCAGSSTPWDIPLGPNEVGYWGQGTQVTVSS

SEQ ID NO: 333 - protein, 10 aa, Artificial Sequence
  Synthetic polypeptide-alpha-HER2 1145 sdAb sdCDR1
GRIFKNYPMG

SEQ ID NO: 334 - protein, 17 aa, Artificial Sequence
  Synthetic polypeptide-alpha-HER2 1145 sdAb sdCDR2
AITSSGGTTHYRDSVKG

SEQ ID NO: 335 - protein, 16 aa, Artificial Sequence
  Synthetic polypeptide-alpha-HER2 1145 sdAb sdCDR3
SSTPWDIPLGPNEVGY

SEQ ID NO: 336 - protein, 125 aa, Artificial Sequence
  Synthetic polypeptide-alpha-HER2 1146 sdAb
QVQLQESGGGLVQAGGSLRLSCAASGRIFKNYPMGWFRQPPGKDREFVAAITSSGGTTPYRDSVKGRFTISRDNAKNTVYLQMNSLKPEDTAVYYCAGSSTPWDIPLGPNEVGFWGQGTQVTVSS

SEQ ID NO: 337 - protein, 10 aa, Artificial Sequence
  Synthetic polypeptide-alpha-HER2 1146 sdAb sdCDR1
GRIFKNYPMG

SEQ ID NO: 338 - protein, 17 aa, Artificial Sequence
  Synthetic polypeptide-alpha-HER2 1146 sdAb sdCDR2
AITSSGGTTPYRDSVKG

SEQ ID NO: 339 - protein, 16 aa, Artificial Sequence
  Synthetic polypeptide-alpha-HER2 1146 sdAb sdCDR3
SSTPWDIPLGPNEVGF

SEQ ID NO: 340 - protein, 115 aa, Artificial Sequence
  Synthetic polypeptide-alpha-HER2 1149 sdAb
QVQLQESGGGLVQAGGSLRLSCVASGNIFGFNVMGWYRQVPGKQRELVAISTGGRSTDYADSAKGRFTISRNNAKDTVYLQMNSLKPEDTAVYYCKMTMASSDYWGQGTQVTVSS

SEQ ID NO: 341 - protein, 10 aa, Artificial Sequence
  Synthetic polypeptide-alpha-HER2 1149 sdAb sdCDR1
GNIFGFNVMG

SEQ ID NO: 342 - protein, 16 aa, Artificial Sequence
  Synthetic polypeptide-alpha-HER2 1149 sdAb sdCDR2
ISTGGRSTDYADSAKG

SEQ ID NO: 343 - protein, 7 aa, Artificial Sequence
  Synthetic polypeptide-alpha-HER2 1149 sdAb sdCDR3
TMASSDY

SEQ ID NO: 344 - protein, 115 aa, Artificial Sequence
  Synthetic polypeptide-alpha-HER2 1150 sdAb
QVQLQESGGGLVQAGGSLRLSCVASGNIFGFNVMGWYRQVPGKQRELVAISTGGGSTNYADSAKGRFTVSRNNAKDTVYLQMNSLKPEDTAVYYCKMTMASVDYWGQGTQVTVSS

SEQ ID NO: 345 - protein, 10 aa, Artificial Sequence
  Synthetic polypeptide-alpha-HER2 1150 sdAb sdCDR1
GNIFGFNVMG

SEQ ID NO: 346 - protein, 16 aa, Artificial Sequence
  Synthetic polypeptide-alpha-HER2 1150 sdAb sdCDR2
ISTGGGSTNYADSAKG

SEQ ID NO: 347 - protein, 7 aa, Artificial Sequence
  Synthetic polypeptide-alpha-HER2 1150 sdAb sdCDR3
TMASVDY

SEQ ID NO: 348 - protein, 119 aa, Artificial Sequence
  Synthetic polypeptide-alpha-HER2 1156 sdAb
QVQLQESGGGLVQAGGSLKVSCAASRNIGSNYAVGWFRQAPGKEREPVAALRWTGSIIGYDDSLRGRFTISKDDAKNTVYLQMNSLKPEDTGVYYCAARILDRSSYDYWGQGTQVTVSS

SEQ ID NO: 349 - protein, 10 aa, Artificial Sequence
  Synthetic polypeptide-alpha-HER2 1156 sdAb sdCDR1
RNIGSNYAVG

SEQ ID NO: 350 - protein, 17 aa, Artificial Sequence
  Synthetic polypeptide-alpha-HER2 1156 sdAb sdCDR2
ALRWTGSIIGYDDSLRG

SEQ ID NO: 351 - protein, 10 aa, Artificial Sequence
  Synthetic polypeptide-alpha-HER2 1156 sdAb sdCDR3
RILDRSSYDY

SEQ ID NO: 352 - protein, 125 aa, Artificial Sequence
  Synthetic polypeptide-alpha-HER2 1158 sdAb
QVQLQESGGGLVQAGGSLRLSCTASGRTFSSYAMGWFRQAPEKEREFVAAITWSGGTTHYAESVKGRFTISRDNAKNTVYLQMNTLEPEDTAVYYCAARINYSTISSNEKMYHYWGQGTQVTVSS

SEQ ID NO: 353 - protein, 10 aa, Artificial Sequence
  Synthetic polypeptide-alpha-HER2 1158 sdAb sdCDR1
GRTFSSYAMG

SEQ ID NO: 354 - protein, 17 aa, Artificial Sequence
  Synthetic polypeptide-alpha-HER2 1158 sdAb sdCDR2
AITWSGGTTHYAESVKG

SEQ ID NO: 355 - protein, 16 aa, Artificial Sequence
  Synthetic polypeptide-alpha-HER2 1158 sdAb sdCDR3
RINYSTISSNEKMYHY

SEQ ID NO: 356 - protein, 123 aa, Artificial Sequence
  Synthetic polypeptide-alpha-HER2 1159 sdAb
QVQLQESGGGLVQAGGSLRLSCATSGRTFSSLAMGWFRQAPGKERELVAGISWSGSTTYYADSVKGRFAISRDNAKNTVYLQMNSLKPEDTAVYYCAARTSSIATIGREYDYWGQGTQVTVSS

SEQ ID NO: 357 - protein, 10 aa, Artificial Sequence
  Synthetic polypeptide-alpha-HER2 1159 sdAb sdCDR1
GRTFSSLAMG

SEQ ID NO: 358 - protein, 17 aa, Artificial Sequence
  Synthetic polypeptide-alpha-HER2 1159 sdAb sdCDR2
GISWSGSTTYYADSVKG

SEQ ID NO: 359 - protein, 14 aa, Artificial Sequence
  Synthetic polypeptide-alpha-HER2 1159 sdAb sdCDR3
RTSSIATIGREYDY

SEQ ID NO: 360 - protein, 119 aa, Artificial Sequence
  Synthetic polypeptide-alpha-HER2 1160 sdAb
QVQLQESGGGLVQAGGSLRLSCAASGRTFSNERLGWFRQAPGKERELVAAIRWSGVIIGYADSVRGRFTISRDNAKSTVYLQMNSLRPEDTAVYFCAADRGVYGTWDYWGQGTQVTVSS

SEQ ID NO: 361 - protein, 10 aa, Artificial Sequence
  Synthetic polypeptide-alpha-HER2 1160 sdAb sdCDR1
GRTFSNERLG

SEQ ID NO: 362 - protein, 17 aa, Artificial Sequence
  Synthetic polypeptide-alpha-HER2 1160 sdAb sdCDR2
AIRWSGVIIGYADSVRG

SEQ ID NO: 363 - protein, 10 aa, Artificial Sequence
  Synthetic polypeptide-alpha-HER2 1160 sdAb sdCDR3
DRGVYGTWDY

SEQ ID NO: 364 - protein, 113 aa, Artificial Sequence
  Synthetic polypeptide-alpha-HER2 1161 sdAb
QVQLQESGGGLVQPGKSLRLSCAVSGDIFGTYGMAWYRQAPGKQREPVASISIGGIINYADSVKGRFTISRDNVKNTVYLQMNSLKPEDTAVYYCKLLDWNYWGRGTQVTVSS

SEQ ID NO: 365 - protein, 10 aa, Artificial Sequence
  Synthetic polypeptide-alpha-HER2 1161 sdAb sdCDR1
GDIFGTYGMA

SEQ ID NO: 366 - protein, 16 aa, Artificial Sequence
  Synthetic polypeptide-alpha-HER2 1161 sdAb sdCDR2
SISIGGIINYADSVKG

SEQ ID NO: 367 - protein, 5 aa, Artificial Sequence
  Synthetic polypeptide-alpha-HER2 1161 sdAb sdCDR3
LDWNY

SEQ ID NO: 368 - protein, 127 aa, Artificial Sequence
  Synthetic polypeptide-alpha-HER2 1162 sdAb
QVQLQESGGGLVQAGGSLRLSCAASGRPFSGNAMGWFRQAPGKEREFVAAISWSGDSTYYADSVKGRFTISRDNDENTAYLQMNSLKPEDTAVYYCAATRSFYSRTYYTRPSDYNYWGPGTQVTVSS

SEQ ID NO: 369 - protein, 10 aa, Artificial Sequence
  Synthetic polypeptide-alpha-HER2 1162 sdAb sdCDR1
GRPFSGNAMG

SEQ ID NO: 370 - protein, 17 aa, Artificial Sequence
  Synthetic polypeptide-alpha-HER2 1162 sdAb sdCDR2
AISWSGDSTYYADSVKG

SEQ ID NO: 371 - protein, 18 aa, Artificial Sequence
  Synthetic polypeptide-alpha-HER2 1162 sdAb sdCDR3
TRSFYSRTYYTRPSDYNY

SEQ ID NO: 372 - protein, 125 aa, Artificial Sequence
  Synthetic polypeptide-alpha-HER2 1163 sdAb
QVQLQESGGGLVQAGGSLRLSCAASGRRFNNYFMGWFRQAPGKEREFVALLRESGDNTYYTNSVKGRFTISRDNARNTVYLQMNSLKPEDTAVYYCAARLDTGFVLYSANSYAYWGQGTQVTVSS

SEQ ID NO: 373 - protein, 10 aa, Artificial Sequence
  Synthetic polypeptide-alpha-HER2 1163 sdAb sdCDR1
GRRFNNYFMG

SEQ ID NO: 374 - protein, 17 aa, Artificial Sequence
  Synthetic polypeptide-alpha-HER2 1163 sdAb sdCDR2
LLRESGDNTYYTNSVKG

SEQ ID NO: 375 - protein, 16 aa, Artificial Sequence
  Synthetic polypeptide-alpha-HER2 1163 sdAb sdCDR3
RLDTGFVLYSANSYAY

SEQ ID NO: 376 - protein, 118 aa, Artificial Sequence
  Synthetic polypeptide-alpha-HER2 h1139 sdAb
QVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSS

SEQ ID NO: 377 - protein, 10 aa, Artificial Sequence
  Synthetic polypeptide-alpha-HER2 h1139 sdAb sdCDR1
GFTFDDYAMS

SEQ ID NO: 378 - protein, 17 aa, Artificial Sequence
  Synthetic polypeptide-alpha-HER2 h1139 sdAb sdCDR2
GINWSGTHTRYADSVKG

SEQ ID NO: 379 - protein, 9 aa, Artificial Sequence
  Synthetic polypeptide-alpha-HER2 h1139 sdAb sdCDR3
GFAADSRST

SEQ ID NO: 380 - protein, 119 aa, Artificial Sequence
  Synthetic polypeptide-alpha-HER2 h1156 sdAb
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSS

SEQ ID NO: 381 - protein, 10 aa, Artificial Sequence
  Synthetic polypeptide-alpha-HER2 h1156 sdAb sdCDR1
RNIGSNYAVG

SEQ ID NO: 382 - protein, 17 aa, Artificial Sequence
  Synthetic polypeptide-alpha-HER2 h1156 sdAb sdCDR2
ALRWTGSIIGYDDSLRG

SEQ ID NO: 383 - protein, 10 aa, Artificial Sequence
  Synthetic polypeptide-alpha-HER2 h1156 sdAb sdCDR3
RILDRSSYDY

SEQ ID NO: 384 - protein, 123 aa, Artificial Sequence
  Synthetic polypeptide-alpha-HER2 h1159 sdAb
QVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSS

SEQ ID NO: 385 - protein, 10 aa, Artificial Sequence
  Synthetic polypeptide-alpha-HER2 h1159 sdAb sdCDR1
GRTFSSLAMG

SEQ ID NO: 386 - protein, 17 aa, Artificial Sequence
  Synthetic polypeptide-alpha-HER2 h1159 sdAb sdCDR2
GISWSGSTTYYADSVKG

SEQ ID NO: 387 - protein, 14 aa, Artificial Sequence
  Synthetic polypeptide-alpha-HER2 h1159 sdAb sdCDR3
RTSSIATIGREYDY

SEQ ID NO: 388 - protein, 127 aa, Artificial Sequence
  Synthetic polypeptide-alpha-HER2 h1162 sdAb
QVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSS

SEQ ID NO: 389 - protein, 10 aa, Artificial Sequence
  Synthetic polypeptide-alpha-HER2 h1162 sdAb sdCDR1
GRPFSGNAMG

SEQ ID NO: 390 - protein, 17 aa, Artificial Sequence
  Synthetic polypeptide-alpha-HER2 h1162 sdAb sdCDR2
AISWSGDSTYYADSVKG

SEQ ID NO: 391 - protein, 18 aa, Artificial Sequence
  Synthetic polypeptide-alpha-HER2 h1162 sdAb sdCDR3
TRSFYSRTYYTRPSDYNY

SEQ ID NO: 392 - protein, 126 aa, Artificial Sequence
  Synthetic polypeptide-alpha-EpCAM h664 sdAb
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSS

SEQ ID NO: 393 - protein, 10 aa, Artificial Sequence
  Synthetic polypeptide-alpha-EpCAM h664 sdAb sdCDR1
GRTLDNYDMG

SEQ ID NO: 394 - protein, 17 aa, Artificial Sequence
  Synthetic polypeptide-alpha-EpCAM h664 sdAb sdCDR2
AISWSGGSTDYAYSVKG

SEQ ID NO: 395 - protein, 17 aa, Artificial Sequence
  Synthetic polypeptide-alpha-EpCAM h664 sdAb sdCDR3
DLRFTGGDTMTPETYDY

(19) United States
(12) Patent Application Publication
(10) Pub. No.: US 2023/0312715 A1
(43) Pub. Date: Oct. 5, 2023

(54) CONSTRAINED CONDITIONALLY ACTIVATED BINDING PROTEIN CONSTRUCTS WITH HUMAN SERUM ALBUMIN DOMAINS

(71) Applicant: Takeda Pharmaceutical Company Limited, Osaka-shi, Osaka (JP)

(72) Inventors: Robert B. DuBridge, Belmont, CA (US); Daniel J. Hicklin, Brisbane, CA (US); Patricia A. Culp, Oakland, CA (US)

(73) Assignee: Takeda Pharmaceutical Company Limited, Osaka-shi, Osaka (JP)

(21) Appl. No.: 18/024,597

(22) PCT Filed: Sep. 3, 2021
(86) PCT No.: PCT/US2021/049109

§ 371 Date: Mar. 3, 2023

Related U.S. Application Data
(60) Provisional application No. 63/074,699, filed on Sep. 4, 2020.

(51) Int. Cl. C07K 16/28; A61P 35/00; C07K 16/30

(52) U.S. Cl. CPC C07K 16/2809; A61P 35/00; C07K 16/30; C07K 2319/31; C07K 2317/565; C07K 2317/62; C07K 2317/569; C07K 2319/50; A61K 38/00

(57) ABSTRACT

Provided herein are Conditional Bispecific Redirected Activation constructs, or COBRAs, that are administered in an active pro-drug format, which includes a human semm albumin (HSA) domain that increases its semm half-life. Upon exposure to tumor proteases, the constructs are cleaved and activated, such that they can bind both tumor target antigens (TTAs) as well as CD3, and thus recruiting T cells expressing CD3 to the tumor, resulting in treatment.

### CROSS REFERENCE TO RELATED APPLICATIONS

This application claims priority under 35 U.S.C. § 119(e) to U.S. Provisional Application No. 63/074,699 filed Sep. 4, 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 27, 2021, is named 118459-5013-WO_SL.txt and is 402,596 bytes in size.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

The use of intact monoclonal antibodies (mAb), which provide superior binding specificity and affinity for a tumor-associated antigen, have been successfully applied in the area of cancer treatment and diagnosis. However, the large size of intact mAbs, their poor bio-distribution, low potency and long persistence in the blood pool have limited their clinical applications. For example, intact antibodies can exhibit specific accumulation within the tumor area. In biodistribution studies, an inhomogeneous antibody distribution with primary accumulation in the peripheral regions is noted when precisely investigating the tumor. Due to tumor necrosis, inhomogeneous antigen distribution and increased interstitial tissue pressure, it is not possible to reach central portions of the tumor with intact antibody constructs. In contrast, smaller antibody fragments show rapid tumor localization, penetrate deeper into the tumor, and also, are removed relatively rapidly from the bloodstream.

However, many antibodies, including scFvs and other constructs, show “on target/off tumor” effects, wherein the molecule is active on non-tumor cells, causing side effects, some of which can be toxic. The present invention is related to novel constructs that are selectively activated in the presence of tumor proteases.

### SUMMARY OF THE INVENTION

In one aspect, provided herein is a fusion protein comprising, from N- to C-terminal: (a) a first single domain antigen binding domain (sdABD) that binds to a human tumor target antigen (TTA) (sdABD-TTA); (b) a first domain linker; (c) a constrained Fv domain comprising: (1) a first variable heavy domain comprising a vhCDR1, vhCDR2 and vhCDR3; (2) a constrained non-cleavable linker (CNCL); and (3) a first variable light domain comprising v1CDR1, v1CDR2 and v1CDR3; (d) a second domain linker; (e) a second sdABD-TTA; (f) a cleavable linker (CL); (g) a constrained pseudo Fv domain comprising: (1) a first pseudo variable light domain; (2) a non-cleavable linker (NCL); and (3) a first pseudo variable heavy domain; (h) a third domain linker; and (i) a human serum albumin (HSA) domain; wherein the first variable heavy domain and the first variable light domain of the constrained Fv domain are capable of binding human CD3 but the constrained pseudo Fv domain does not bind CD3; wherein the first variable heavy domain and the first pseudo variable light domain intramolecularly associate to form an inactive Fv domain; and wherein the first variable light domain and the first pseudo variable heavy domain intramolecularly associate to form an inactive Fv domain.

In some embodiments, the first variable heavy domain is N-terminal to the first variable light domain and the pseudo variable light domain is N-terminal to the pseudo variable heavy domain.

In some embodiments, the first variable heavy domain is N-terminal to the first variable light domain and the pseudo variable heavy domain is N-terminal to the pseudo variable light domain.

In some embodiments, the first variable light domain is N-terminal to the first variable heavy domain and the pseudo variable light domain is N-terminal to the pseudo variable heavy domain.

In some embodiments, the first variable light domain is N-terminal to the first variable heavy domain and the pseudo variable heavy domain is N-terminal to the pseudo variable light domain.

In some embodiments, the first TTA and the second TTA are the same. In some embodiments, the first TTA and the second TTA are selected from the group consisting of B7H3, CA9, EGFR, EpCAM, FOLR1, HER2, LyPD3 and Trop2. In some embodiments, the first TTA and second TTA is B7H3. In some embodiments, the first TTA and second TTA is CA9. In some embodiments, the first TTA and second TTA is EGFR. In some embodiments, the first TTA and second TTA is EpCAM. In some embodiments, the first TTA and second TTA is FOLR1. In some embodiments, the first TTA and second TTA is HER2. In some embodiments, the first TTA and second TTA is LyPD3. In some embodiments, the first TTA and second TTA is Trop2.

In some embodiments, the first sdABD-TTA and the second sdABD-TTA are the same. In some embodiments; the first sdABD-TTA and the second sdABD-TTA are different. In some embodiments, the first and the second sdABD-TTAs are selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, and SEQ ID NO:344.

In some embodiments, the first TTA and the second TTA are different. In some embodiments, the first TTA and the second TTA are selected from the group consisting of B7H3, CA9, EGFR, EpCAM, FOLR1, HER2, LyPD3, Trop2, and any combination thereof. In some embodiments, the fusion protein comprises (a) the first TTA is B7H3 and the second TTA is selected from the group consisting of B7H3, CA9 (CAIX); EGFR, EpCAM, FOLR1, HER2, LyPD3 and Trop2; (b) the first TTA is CA9 and the second TTA is selected from the group consisting of B7H3, CA9 (CAIX), EGFR, EpCAM, FOLR1, HER2, LyPD3 and Trop2; (c) the first TTA is EGFR and the second TTA is selected from the group consisting of B7H3, CA9 (CAIX), EGFR, EpCAM, FOLR1, HER2, LyPD3 and Trop2; (d) the first TTA is EpCAM and the second TTA is selected from the group consisting of B7H3, CA9 (CAIX), EGFR, EpCAM, FOLR1, HER2, LyPD3 and Trop2; (e) the first TTA is FOLR1 and the second TTA is selected from the group consisting of B7H3, CA9 (CAIX), EGFR, EpCAM, FOLR1, HER2, LyPD3 and Trop2; (f) the first TTA is HER2 and the second TTA is selected from the group consisting of B7H3, CA9 (CAIX), EGFR, EpCAM, FOLR1, HER2, LyPD3 and Trop2; (g) the first TTA is LyPD3 and the second TTA is selected from the group consisting of B7H3, CA9, EGFR, EpCAM, FOLR1, HER2, LyPD3 and Trop2; or (h) the first TTA is Trop2 and the second TTA is selected from the group consisting of B7H3, CA9 (CAIX), EGFR, EpCAM, FOLR1, HER2, LyPD3 and Trop2. In some embodiments, the fusion protein comprises (a) the first TTA is selected from the group consisting of B7H3, CA9 (CAIX), EGFR, EpCAM, FOLR1, HER2, LyPD3 and Trop2 and the second TTA is B7H3; (b) the first TTA is selected from the group consisting of B7H3, CA9 (CAIX), EGFR, EpCAM, FOLR1, HER2, LyPD3 and Trop2 and the second TTA is CA9 (CAIX); (c) the first TTA is selected from the group consisting of B7H3, CA9 (CAIX); EGFR, EpCAM, FOLR1, HER2, LyPD3 and Trop2 and the second TTA is EGFR; (d) the first TTA is selected from the group consisting of B7H3, CA9 (CAIX), EGFR, EpCAM, FOLR1, HER2, LyPD3 and Trop2 and the second TTA is EpCAM; (e) the first TTA is selected from the group consisting of B7H3, CA9 (CAIX), EGFR, EpCAM, FOLR1, HER2, LyPD3 and Trop2 and the second TTA is FOLR1; (f) the first TTA is selected from the group consisting of B7H3, CA9 (CAIX), EGFR, EpCAM, FOLR1, HER2, LyPD3 and Trop2 and the second TTA is HER2; (g) the first TTA is selected from the group consisting of B7H3, CA9 (CAIX), EGFR, EpCAM, FOLR1, HER2, LyPD3 and Trop2 and the second TTA is LyPD3; or (h) the first TTA is selected from the group consisting of B7H3, CA9 (CAIX); EGFR, EpCAM, FOLR1, HER2, LyPD3 and Trop2 and the second TTA is Trop2. In some embodiments, the first and/or the second sdABD-TTAs are selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID

NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, SEQ ID NO:344, and any combination thereof.

In some embodiments, the first HSA domain comprises an amino acid sequence having at least 90% sequence identity to SEQ ID NO:117.

In some embodiments, the first HSA domain comprises the amino acid sequence of SEQ ID NO:117.

In some embodiments, the first cleavable linker is cleaved by a human protease selected from the group consisting of MMP2, MMP9, meprin A, meprin B, cathepsin S, cathepsin K, cathespin L, granzymeB, uPA, kallekriein7, matriptase and thrombin.

In some embodiments, the first cleavable linker comprises an amino acid sequence selected from the group consisting of SEQ ID NOS:152-225.

In some embodiments, the first domain linker is a flexible linker.

In some embodiments, the first flexible linker comprises an amino acid sequence selected from the group consisting of (GS)n, (GGS)n, (GGGS)n (SEQ ID NO:244), (GGSG)n (SEQ ID NO:245), (GGSGG)n (SEQ ID NO:246), or (GGGGS)n (SEQ ID NO:247), wherein n is 1, 2, 3, 4, 5, 6, 7, 8, 9, or 10.

In some embodiments, the first variable heavy domain comprises a vhCDR1 of SEQ ID NO:135, a vhCDR2 of SEQ ID NO:136 and a vhCDR3 of SEQ ID NO:137.

In some embodiments, the first variable light domain comprises a v1CDR1 of SEQ ID NO:119, a v1CDR2 of SEQ ID NO:120 and a v1CDR3 of SEQ ID NO:121.

In some embodiments, the first variable heavy domain comprises the amino acid sequence of SEQ ID NO:134 and the first variable light domain comprises the amino acid sequence of SEQ ID NO:118.

In some embodiments, the first constrained pseudo FAT domain comprises the first pseudo variable light domain having the amino acid sequence of SEQ ID NO:122 and the first pseudo variable heavy domain having the amino acid sequence of SEQ ID NO:138.

In some embodiments, the first constrained pseudo Fv domain comprises the first pseudo variable light domain having the amino acid sequence of SEQ ID NO:126 and the first pseudo variable heavy domain having the amino acid sequence of SEQ ID NO:142.

In some embodiments, the first constrained pseudo Fv domain comprises the first pseudo variable light domain having the amino acid sequence of SEQ ID NO:130 and the first pseudo variable heavy domain having the amino acid sequence of SEQ ID NO:146.

In some embodiments, the fusion protein has an amino acid sequence selected from the group consisting of SEQ ID NOS:226-231 and 235-243.

In some embodiments, the fusion protein has at least a 10%, 20%, 30%, 40%, 50%, 60%, 70%, 80%, or 90% increase in serum half-life relative to a corresponding fusion protein without a half-life extension domain.

In some embodiments, the fusion protein has at least a 100%, 200%, 300%, 400%, 500%, 600%, 700%, 800%, or 900% increase in serum half-life relative to a corresponding fusion protein without a half-life extension domain.

In some embodiments, the fusion protein has at least a 1000% increase in serum half-life relative to a corresponding fusion protein without a half-life extension domain.

In some embodiments, the increase in serum half-life is determined using a mouse surrogate for evaluating pharmacokinetics of a human serum albumin domain. In some embodiments, the mouse surrogate is an Alb^{-/-} hFcRn humanized mouse. In some embodiments, the Alb^{-/-} hFcRn humanized mouse is a Tg32- Alb^{-/-} mFcRn^{-/-} hFcRn^{Tg/Tg} mouse.

In some embodiments, provided herein is a nucleic acid encoding a fusion protein according to any one described herein. In some embodiments, provided herein is an expression vector comprising any of the nucleic acids described herein.

In some embodiments, provided herein is a host cell comprising any one of the expression vectors described herein. Also provided herein is a method of making a fusion protein comprising (a) culturing any one of the host cells described under conditions wherein the fusion protein is expressed and (b) recovering the fusion protein.

Also provided herein is a method of treating cancer comprising administering any one of the fusion proteins described herein to a subject.

Provided herein is use of any one of the fusion proteins disclosed herein in the manufacture of a medicament for the treatment of cancer.

### BRIEF DESCRIPTION OF THE DRAWINGS

FIG. 1 depicts a schematic of the protease activation of the present invention, referred to herein as “constrained, non-cleavable constructs”, or “CNCL constructs”, also sometimes referred to herein as “dimerization constructs” as discussed herein. Upon cleavage, two prodrug construct splits into four components, two HSA domains linked to two pseudo domains (which may or may not be able to self-associate, depending on the length of the linkers and the inactivating mutations), and two active moieties that self-assemble into a dimeric active moiety that contains four anti-TTA domains (which can be all the same or two are the same and the other two are different). It should be noted that in these format embodiments, the resulting active component is hexavalent: there is bivalent binding to CD3 and quadrivalent binding to the TTA, rendering a bispecific binding protein, although in some cases this hexavalency could be trispecifics, with bivalent binding to CD3, bivalent binding to a first TTA and bivalent binding to a second TTA. FIG. 1 also has the VH and VL of the Fv and iVH and iVL of the pseudo Fv in a specific order, e.g. from N- to C-terminal, VH-linker-VL (and iVL-linker-iVH) although as will be appreciated by those in the art, these can be reversed (VL-linker-VH and iVH-linker-iVL).

FIG. 2A-FIG. 2N depict a number of sequences of the invention. For antigen binding domains, the CDRs are underlined.

FIG. 3A-FIG. 3D depict a number of suitable protease cleavage sites. As will be appreciated by those in the art, these cleavage sites can be used as cleavable linkers. In some embodiments, for example when more flexible cleavable linkers are required, there can be additional amino acids (generally glycines and serines) that are either or both N- and C-terminal to these cleavage sites. Of note is that SEQ ID NO:170 and SEQ ID NO:171 are cleaved by MMP9 slightly faster than SEQ ID NO:s 152-153, and SEQ ID NO:172 is cleaved slower than SEQ ID NOS: 152-153.

FIG. 4A-FIG. 4H show some sequences of the COBRA-HSA constructs of the invention. CDRs are bolded and underlined; non-cleavable linkers are double underlined; cleavable linkers are double underlined and italicized; and junctions between the domains have a slash (“/”).

FIG. 5A-FIG. 5B show that the MMP9 linker is stable in vivo. FIG. 5A shows a schematic diagram of an EGFR hemi-COBRA. NSG mice were administered a single intravenous bolus dose of either Pro40 (MMP9 cleavable), Pro74 (non-cleavable) via the tail vein at a dose level of 0.5 mg/kg. The dose solution for each compound was prepared in a vehicle of 25 mM citric acid, 75 mM L-arginine, 75 mM NaCl and 4% sucrose pH 7.0. Two blood samples were collected at preselected times from each animal, one towards the beginning of the study, collected by orbital bleed or submandibular bleed, and another at the terminal time point by cardiac puncture. The time points for blood collection were 0.083, 1, 6, 24, 72, and 168 hrs. Plasma was prepared from each individual blood sample using K₂ EDTA tubes. Concentrations were determined using an MSD assay with a MAb specific to the anti-HSA sdABD and detected with the EGFR extracellular domain.

FIG. 6A-FIG. 6B show that the COBRA-HSA fusion proteins of the invention are conditionally activated. FIG. 6A shows a schematic diagram of the Pro186 and Pro817 constructs. Pro201 is the active dimer from Pro186 and Pro214 is Pro186 with a non-cleavable linker instead of the MMP9 linker; the sequences are shown in FIGS. 4A-4H.

FIG. 7A-FIG. 7B show a schematic diagram of the Pro817 construct and an SDS-PAGE of the cleavage products of Pro817.

FIG. 8 shows the domain schematics for some of the COBRA-HSA constructs of the invention. The trispecific construct uses ABDs to B7H3 and EpCAM as exemplary TTAs in addition to the CD3 ABDs, although as will be appreciated by those in the art, any two TTA ABDs can be used as described herein. The tetraspecific construct uses ABDs to B7H3 and EpCAM as exemplary TTAs in addition to the CD3 ABDs and an anti-HSA ABD, although as will be appreciated by those in the art, any two TTA ABDs can be used as described herein.

FIG. 9 shows that the COBRA-HSA fusion proteins are conditionally activated in a T cell cytotoxicity assay. Pro976 is Pro817 with a non-cleavable linker instead of the MMP9 linker. The amino acid sequences of the COBRA-HSA fusion proteins are provided in FIGS. 4 and 15.

FIG. 10 demonstrates half-life extension of the COBRA-HSA fusion protein, Pro817, in mice, compared to Pro1017, a COBRA with no half-life extension moiety.

FIG. 11 shows an SDS-PAGE of the cleavage products of Pro1019, a COBRA with a mouse serum albumin domain (COBRA-MSA) fusion protein, Pro186, a COBRA-anti-HSA protein, and Pro1017, a COBRA with no half-life extension moiety.

FIG. 12A-FIG. 12B show that cleaved COBRA-MSA fusion proteins induce cytotoxicity similarly to standard COBRA molecules with anti-HSA and COBRAs with no half-life extension moiety.

FIG. 13 demonstrates half-life extension of the COBRA-MSA fusion protein, Pro1019, in mice, compared to Pro1017, a COBRA with no half-life extension moiety.

FIG. 14A-FIG. 14C show that COBRA-MSA fusion proteins have similar anti-tumor activity in vivo to COBRA molecules containing anti-HSA moieties and are more active in vivo than COBRA molecules without half-life extension. Shown are tumor volumes of HT29-bearing mice implanted with human T cells and dosed with MSA-fusion COBRA. The number of tumor-free animals in each group at the end of the study is indicated in the parentheses.

FIG. 15A-FIG. 15I depict amino acid sequences of humanized anti-HER2, anti-CA9 and anti-B7H3 COBRA-HSA fusion proteins. FIGS. 15A-15D show humanized anti-HER2 COBRA-HSA fusion proteins, in particular, Pro1109-HSA in FIG. 15A, Pro1111-HSA in FIG. 15B, Pro1117-HSA in FIG. 15C and Pro1118-HSA in FIG. 15D. FIGS. 15E-15H show humanized anti-CA9 COBRA-HSA fusion proteins, in particular, Pro518-HSA in FIG. 15E, Pro519-HSA in FIG. 15F, Pro516-HSA in FIG. 15G, and Pro517-HSA in FIG. 15H. FIG. 15I shows an humanized anti-B7H3 COBRA-HSA fusion protein Pro974. CDRs are bolded and underlined; non-cleavable linkers are double underlined; cleavable linkers are double underlined and italicized; and junctions between the domains have a slash (“/”).

### DETAILED DESCRIPTION OF THE INVENTION

I. Introduction

The present invention is directed to methods of reducing the toxicity and side effects of bispecific antibodies (including antibody-like functional proteins) that bind to important physiological targets such as CD3 and tumor antigens. Many antigen binding proteins, such as antibodies, can have significant off-target side effects, and thus there is a need to only activate the binding capabilities of a therapeutic molecule in the vicinity of the disease tissue, to avoid off-target interactions. Accordingly, the present invention is directed to multivalent conditionally effective (“MCE”) proteins that have a number of functional protein domains. In general, one of these domains is an antigen binding domain (ABD) that will bind a target tumor antigen (TTA), and another is an ABD that will bind a T cell antigen such as CD3 under certain conditions. Additionally, the MCE proteins also include one or more protease cleavage sites. That is, the therapeutic molecules are made in a “pro-drug” like format, wherein the CD3 binding domain is inactive until exposed to a tumor environment. The tumor environment contains proteases, such that upon exposure to the protease, the prodrug is cleaved and becomes active.

This is generally accomplished herein by using proteins that include a “pseudo” variable heavy domain and a “pseudo” variable light domain directed to the T cell antigen such as CD3, that restrain the CD3 Fvs of the MCE into an inactive format as is discussed herein. As the TTA targets the MCE into the proximity of the tumor, the MCE is thus exposed to the protease. Upon cleavage, the active variable heavy domain and the active variable light domain are now able to pair to form one or more active ABDs to CD3 and thus recruit T cells to the tumor, resulting in treatment.

In general, the CD3 binding domain (“Fv”) is in a constrained format, wherein the linker between the active variable heavy domain and the active variable light domain that traditionally form an Fv is too short to allow the two active variable domains to bind each other; this is referred to as “constrained linker”. Rather, in the prodrug (e.g., uncleaved) format, the prodrug polypeptide also comprises a “pseudo Fv domain”. The pseudo Fv domain comprises a variable heavy and light domain, with standard framework regions, but “inert” or “inactive” CDRs. The pseudo Fv domain also has a constrained linker between the inactive variable heavy and inactive variable light domains. Since neither Fv nor pseudo Fv domains can self-assemble due to the steric constraints, there is an intramolecular assembly that pairs the aVL with the iVH and the aVH with the iVL, due to the affinity of the framework regions of each. However, due to the “inert” CDRs of the pseudo domain, the resulting ABDs will not bind CD3, thus preventing off target toxicities. However, in the presence of proteases that are in or near the tumor, the prodrug construct is cleaved such that the pseudo-Fv domain is released from the surface and thus allows the “real” variable heavy and variable light domains to associate intermolecularly (e.g. two cleaved constructs come together), thus triggering active CD3 binding and the resulting tumor efficacy. These constructs are generally referred to herein as COnditional Bispecific Redirected Activation constructs, or “COBRAs™”. The stability of the intramolecular assembly is shown by the conditionality experiments herein, whereby in the absence of protease, the uncleaved constructs have no activity (e.g., no active CD3 binding domain is formed).

Interestingly, for ease of description, while these constructs are all referred to herein as “constrained”, additional work shows that the intramolecular assembly is favored even if one of the Fv domains is not constrained, e.g. one of the domains can have a longer, flexible linker. That is, as shown in FIG. 37, FIG. 38 and FIG. 39 of US Pub. No. 2019/0076524, intramolecular assembly still occurs (e.g. the uncleaved constructs are inactive in the absence of protease cleavage) if only one of the Fv domains, either the one with an active VL and VH, or the pseudo Fv domain, is constrained. However, in the current systems, when both linkers are constrained, the protein has better expression. However, as will be appreciated by those of skill in the art, the constructs herein can have one of these Fv domains with an “unconstrained” or “flexible” linker. For ease of reference, the constructs are shown with both Fv domains in a constrained format.

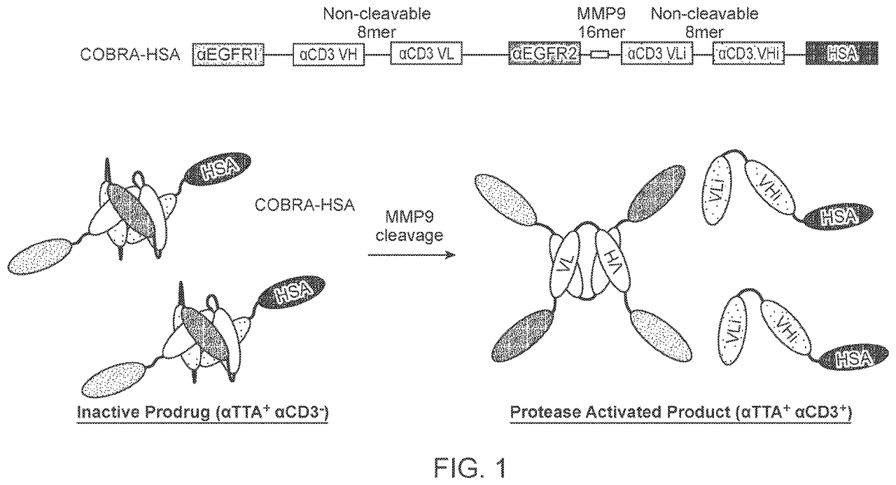

In some embodiments, the prodrug construct is shown in FIG. 1. In this embodiment, the domain linker between the active variable heavy and active light chains is a constrained but not cleavable linker (“CNCL”). In the prodrug format, the inactive VH and VL of the constrained pseudo Fv domain associate with the VH and VL of the constrained Fv domain, such that there is no CD3 binding. However, once cleavage in the tumor environment happens, two different activated proteins, each comprising an active variable heavy and light domain, associate to form two anti-CD3 binding domains.

Accordingly, the formats and constructs of the invention find use in the treatment of disease.

II. Definitions

In order that the application may be more completely understood, several definitions are set forth below. Such definitions are meant to encompass grammatical equivalents.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to CD3 and target tumor antigens (TTAs) such as target cell receptors, as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including Fc parent or precursors) as used herein is meant a polypeptide that is subsequently modified to generate a variant. Such parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an unmodified Fc polypeptide that is modified to generate a variant, and by “parent antibody” as used herein is meant an unmodified antibody that is modified to generate a variant antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein.

By “target cell” as used herein is meant a cell that expresses a target antigen. Generally, for the purposes of the invention, target cells are either tumor cells that express TTAs or T cells that express the CD3 antigen.

By “Fv” or “Fv domain” or “Fv region” as used herein is meant a polypeptide that comprises the VL and VH domains of an antigen binding domain, generally from an antibody. Fv domains usually form an “antigen binding domain” or “ABD” as discussed herein, if they contain active VH and VL domains (although in some cases, an Fv containing a constrained linker is used; such that an active ABD isn't formed prior to cleavage). As discussed below, Fv domains can be organized in a number of ways in the present invention, and can be “active” or “inactive”, such as in a scFv format, a constrained Fv format, a pseudo Fv format, etc. It should be understood that in the present invention, in some cases an Fv domain is made up of a VH and VL domain on a single polypeptide chain, such as shown in FIG. 1 but with a constrained linker such that an intramolecular ABD cannot be formed. In these embodiments, it is after cleavage that two active ABDs are formed. In some cases an Fv domain is made up of a VH and a VL domain, one of which is inert, such that only after cleavage is an intermolecular ABD formed. As discussed below, Fv domains can be organized in a number of ways in the present invention, and can be “active” or “inactive”, such as in a scFv format, a constrained Fv format, a pseudo Fv format, etc. In addition, as discussed herein, Fv domains containing VH and VL can be/form ABDs, and other ABDs that do not contain VH and VL domains can be formed using sdABDs.

By “variable domain” herein is meant the region of an immunoglobulin that comprises one or more Ig domains substantially encoded by any of the Vκ, Vλ, and/or VH genes that make up the kappa, lambda, and heavy chain immunoglobulin genetic loci respectively. In some cases, a single variable domain, such as a sdFv (also referred to herein as sdABD) can be used.

In embodiments utilizing both variable heavy (VH) and variable light (VL) domains, each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure v1FR1-v1CDR1-v1FR2-v1CDR2-v1FR3-v1CDR3-v1FR4. As is more fully described herein, the vhFR regions and the v1FR regions self-assemble to form Fv domains. In general, in the prodrug formats of the invention, there are “constrained Fv domains” wherein the VH and VL domains cannot self-associate, and “pseudo Fv domains” for which the CDRs do not form antigen binding domains when self-associated.

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the v1CDRs (e.g. v1CDR1, v1CDR2 and v1CDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)).

The present invention provides a large number of different CDR sets. In this case, a “full CDR set” in the context of the anti-CD3 component comprises the three variable light and three variable heavy CDRs, e.g. a v1CDR1, v1CDR2, v1CDR3, vhCDR1, vhCDR2 and vhCDR3. As will be appreciated by those in the art, each set of CDRs, the VH and VL CDRs, can bind to antigens, both individually and as a set. For example, in constrained Fv domains, the vhCDRs can bind, for example to CD3 and the v1CDRs can bind to CD3, but in the constrained format they cannot bind to CD3.

In the context of a single domain ABD (“sdABD”) such as are generally used herein to bind to target tumor antigens (TTA), a CDR set is only three CDRs; these are sometimes referred to in the art as “VHH” domains as well.

These CDRs can be part of a larger variable light or variable heavy domain, respectfully. In addition, as more fully outlined herein, the variable heavy and variable light domains can be on separate polypeptide chains or on a single polypeptide chain in the case of scFv sequences, depending on the format and configuration of the moieties herein.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The variable heavy and variable light domains of the invention can be “active” or “inactive”.

As used herein, “inactive VH” (“iVH”) and “inactive VL” (“iVL”) refer to components of a pseudo Fv domain, which, when paired with their cognate VL or VH partners, respectively, form a resulting VH/VL pair that does not specifically bind to the antigen to which the “active” VH or “active” VL would bind were it bound to an analogous VL or VH, which was not “inactive”. Exemplary “inactive VH” and “inactive VL” domains are formed by mutation of a wild type VH or VL sequence as more fully outlined below. Exemplary mutations are within CDR1, CDR2 or CDR3 of VH or VL. An exemplary mutation includes placing a domain linker within CDR2, thereby forming an “inactive VH” or “inactive VL” domain. In contrast, an “active VH” or “active VL” is one that, upon pairing with its “active” cognate partner, i.e., VL or VH, respectively, is capable of specifically binding to its target antigen. Thus, it should be understood that a pseudo Fv can be a VH/iVL pair, a iVH/VL pair, or a iVH/iVL pair.

In contrast, as used herein, the term “active” refers to a CD3 binding domain that is capable of specifically binding to CD3. This term is used in two contexts: (a) when referring to a single member of an Fv binding pair (i.e., VH or VL), which is of a sequence capable of pairing with its cognate partner and specifically binding to CD3; and (b) the pair of cognates (i.e., VH and VL) of a sequence capable of specifically binding to CD3. An exemplary “active” VH, VL or VH/VL pair is a wild type or parent sequence.

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is human CDR.

The term “binding domain” characterizes, in connection with the present invention, a domain which (specifically) binds to/interacts with/recognizes a given target epitope or a given target site on the target molecules (antigens), for example: EGFR and CD3, respectively. The structure and function of the target antigen binding domain (recognizing EGFR), and preferably also the structure and/or function of the CD3 binding domain (recognizing CD3), is/are based on the structure and/or function of an antibody, e.g. of a full-length or whole immunoglobulin molecule, including sdABDs. According to the invention, the target antigen binding domain is generally characterized by the presence of three CDRs that bind the target tumor antigen (generally referred to in the art as variable heavy domains, although no corresponding light chain CDRs are present). Alternatively, ABDs to TTAs can include three light chain CDRs (i.e. CDR1, CDR2 and CDR3 of the VL region) and/or three heavy chain CDRs (i.e. CDR1, CDR2 and CDR3 of the VH region). The CD3 binding domain preferably also comprises at least the minimum structural requirements of an antibody which allow for the target binding. More preferably, the CD3 binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that in exemplary embodiments the target antigen and/or CD3 binding domain is produced by or obtainable by phage-display or library screening methods.

By “domain” as used herein is meant a protein sequence with a function, as outlined herein. Domains of the invention include tumor target antigen binding domains (TTA domains), variable heavy domains, variable light domains, linker domains, and half-life extension domains.

By “domain linker” herein is meant an amino acid sequence that joins two domains as outlined herein. Domain linkers can be cleavable linkers, constrained cleavable linkers, non-cleavable linkers, constrained non-cleavable linkers, scFv linkers, etc.

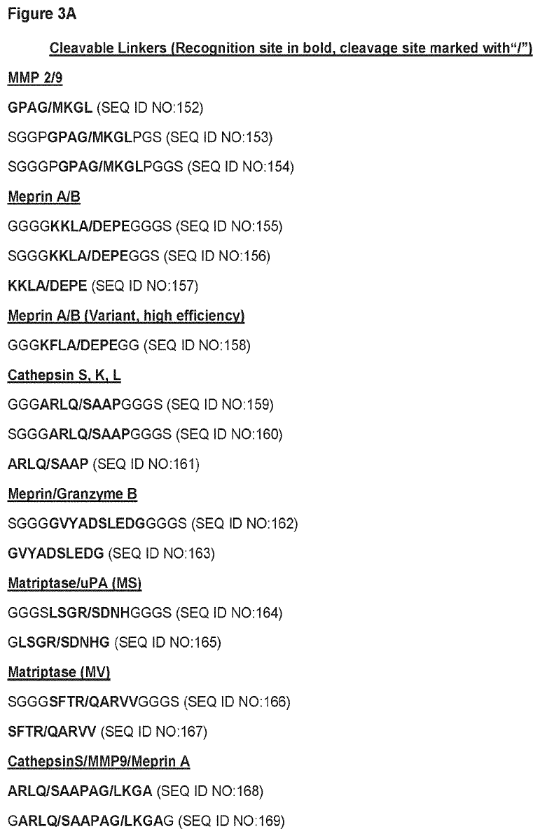

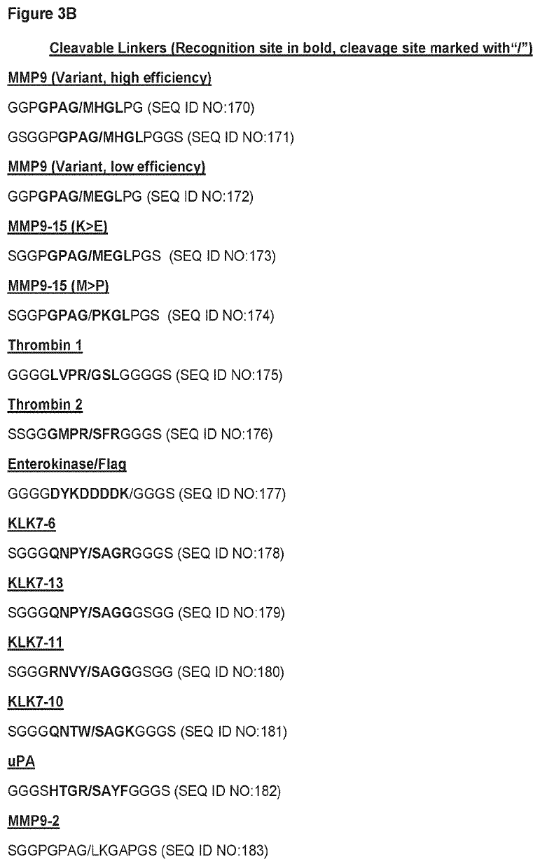

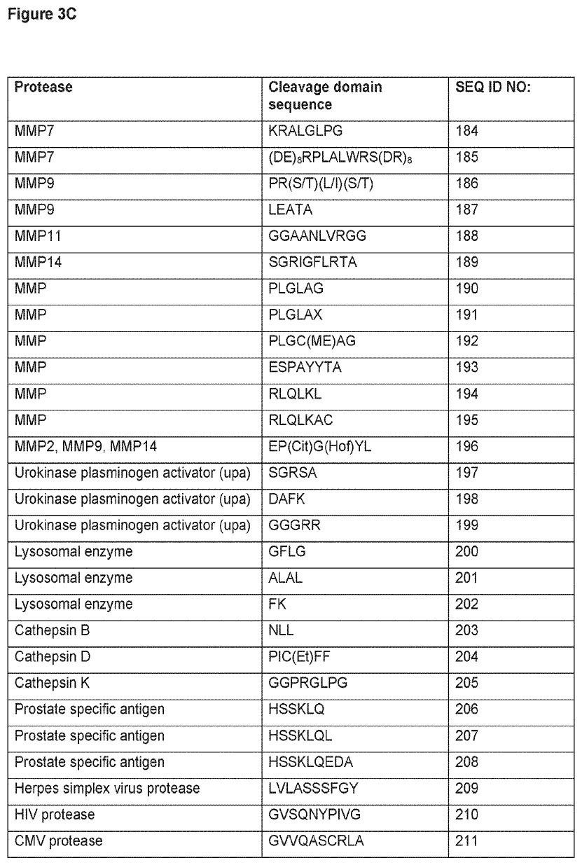

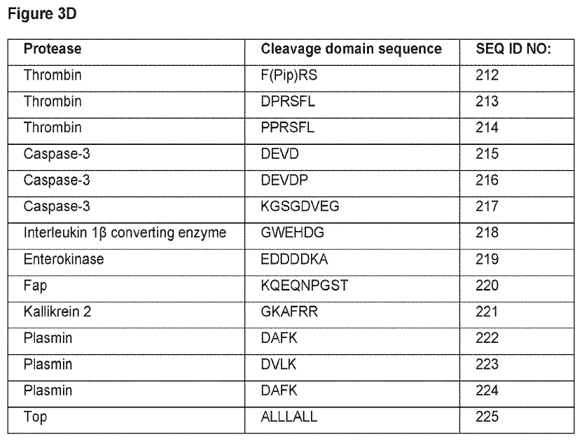

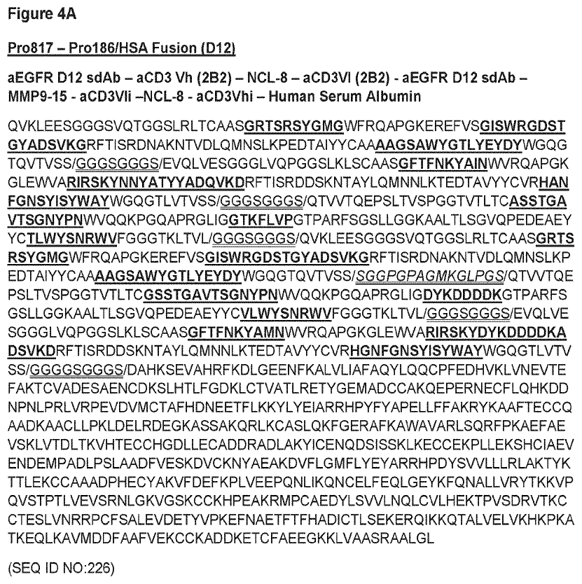

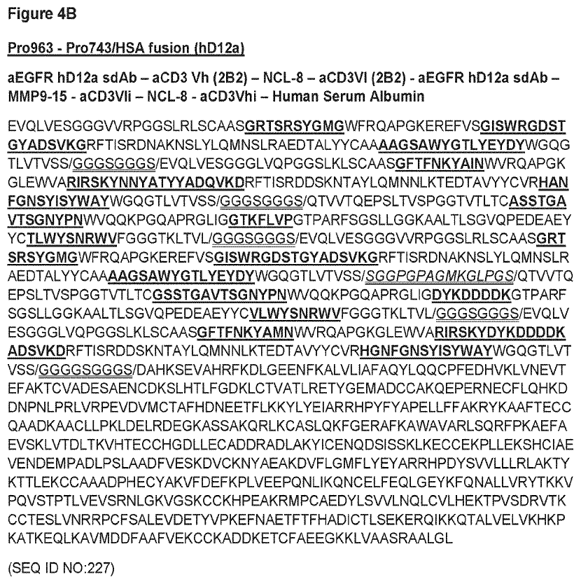

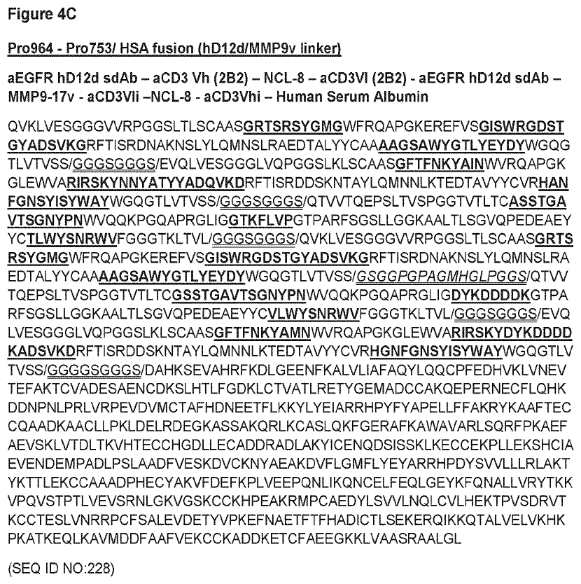

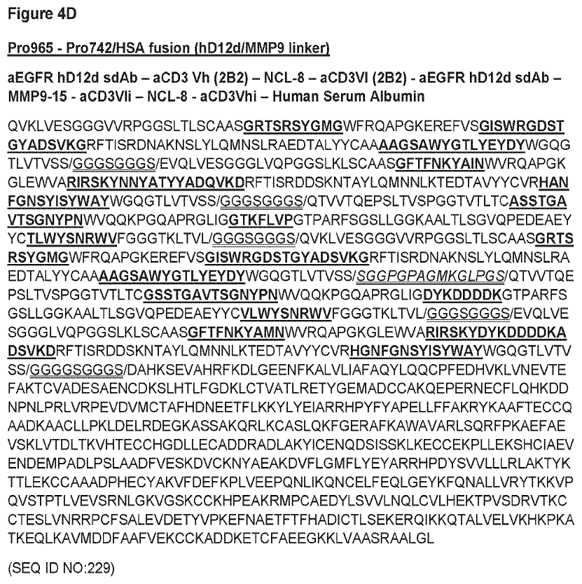

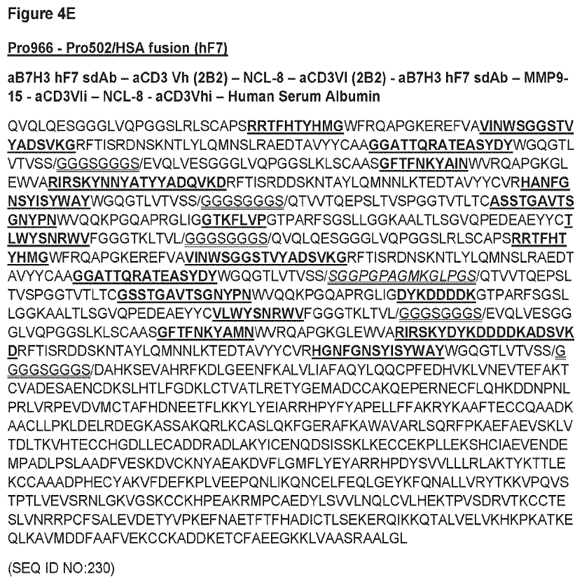

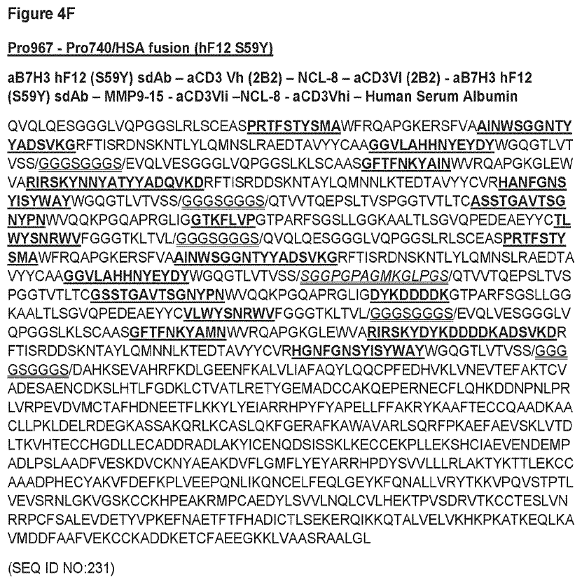

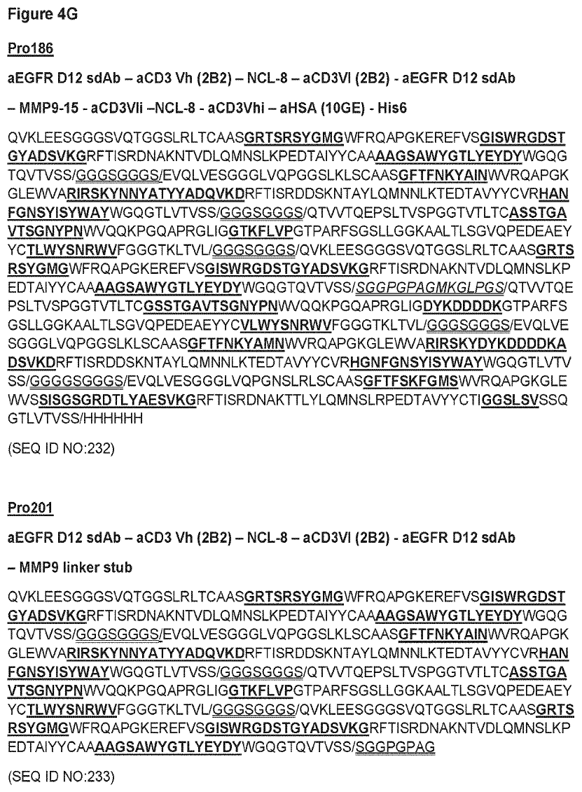

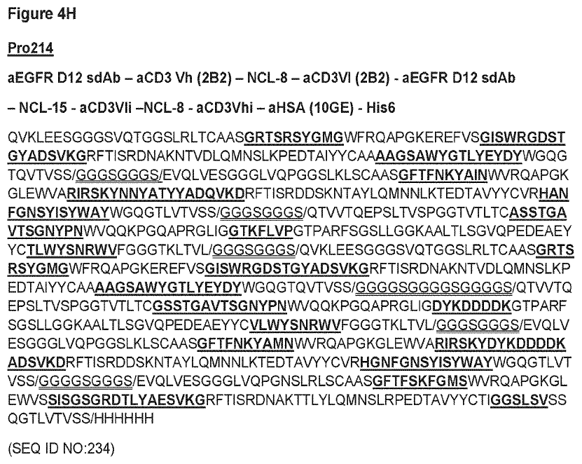

By “cleavable linker” (“CL”) herein is meant an amino acid sequence that can be cleaved by a protease, preferably a human protease in a disease tissue as outlined herein. Cleavable linkers generally are at least 3 amino acids in length, with from 4, 5, 6, 7, 8, 9, 10, 11, 12, 13, 14, 15 or more amino acids finding use in the invention, depending on the required flexibility. A number of cleavable linker sequences are found in FIGS. 3A-3D.

By “non cleavable linker” (“NCL”) herein is meant an amino acid sequence that cannot be cleaved by a human protease under normal physiological conditions.

By “constrained cleavable linker” (“CCL”) herein is meant a short polypeptide that contains a protease cleavage site (as defined herein) that joins two domains as outlined herein in such a manner that the two domains cannot significantly interact with each other until after they reside on different polypeptide chains, e.g. after cleavage. When the CCL joins a VH and a VL domain as defined herein, the VH and VL cannot self-assemble to form a functional Fv prior to cleavage due to steric constraints in an intramolecular way (although they may assemble into pseudo Fv domains in an intermolecular way). Upon cleavage by the relevant protease, the VH and VL can assemble to form an active antigen binding domain in an intermolecular way. In general, CCLs are less than 10 amino acids in length, with 9, 8, 7, 6, 5 and 4 amino acids finding use in the invention. In general, protease cleavage sites generally are at least 4+amino acids in length to confer sufficient specificity, as is shown in FIG. 3.

By “constrained non-cleavable linker” (“CNCL”) herein is meant a short polypeptide that that joins two domains as outlined herein in such a manner that the two domains cannot significantly interact with each other, and that is not significantly cleaved by human proteases under physiological conditions.

By “constrained Fv domain” herein is meant an Fv domain that comprises an active variable heavy domain and an active variable light domain, linked covalently with a constrained linker as outlined herein, in such a way that the active heavy and light variable domains cannot intramolecularly interact to form an active Fv that will bind an antigen such as CD3. Thus, a constrained Fv domain is one that is similar to an scFv but is not able to bind an antigen due to the presence of a constrained linker (although they may assemble intermolecularly with inert variable domains to form pseudo Fv domains).

By “pseudo Fv domain” herein is meant a domain that comprises a pseudo or inactive variable heavy domain or a pseudo or inactive variable light domain, or both, linked using a domain linker (which can be cleavable, constrained, non-cleavable, non-constrained, etc.). The iVH and iVL domains of a pseudo Fv domain do not bind to a human antigen when either associated with each other (iVH/iVL) or when associated with an active VH or VL; thus iVH/iVL, iVH/VL and iVL/VH Fv domains do not appreciably bind to a human protein, such that these domains are inert in the human body.

By “single chain Fv” or “scFv” herein is meant a variable heavy (VH) domain covalently attached to a variable light (VL) domain, generally using a domain linker as discussed herein, to form a scFv or scFv domain. A scFv domain can be in either orientation from N- to C-terminus (VH-linker-VL or VL-linker-VH).

By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). As outlined herein, sdABDs that bind to TTAs are annotated as such (sdABD-TTA for the generic term, or sdABD-EGFR for one that binds to EGFR, sdABD-FOLR1 for one that binds to FOLR1, etc.).

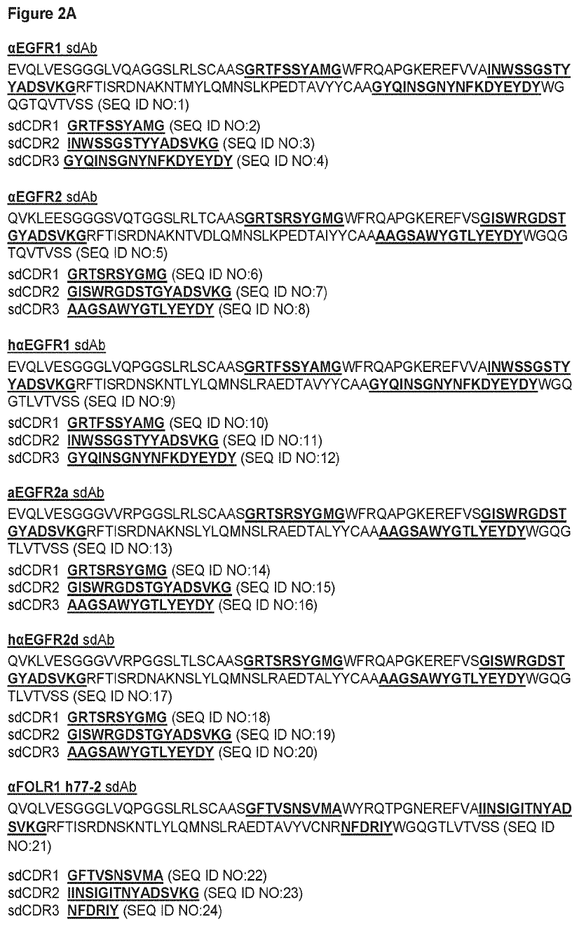

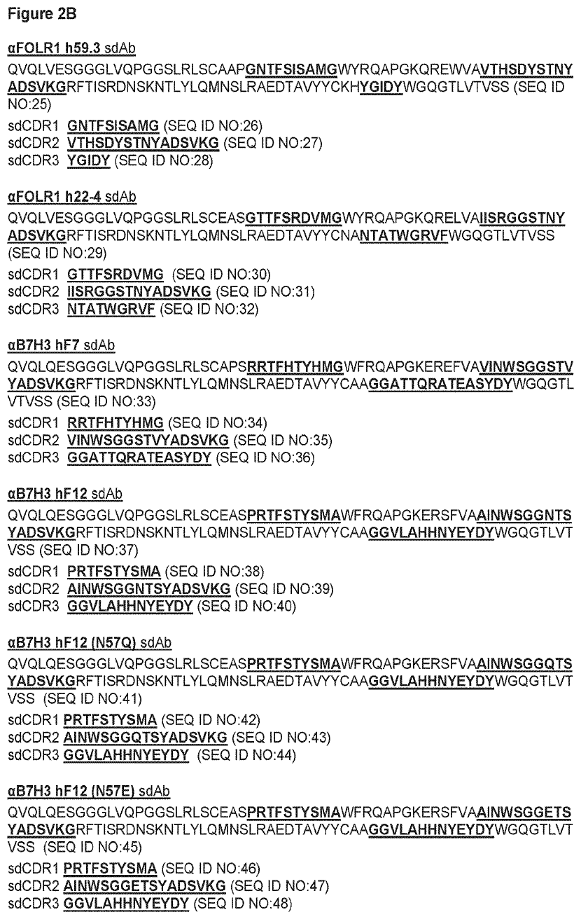

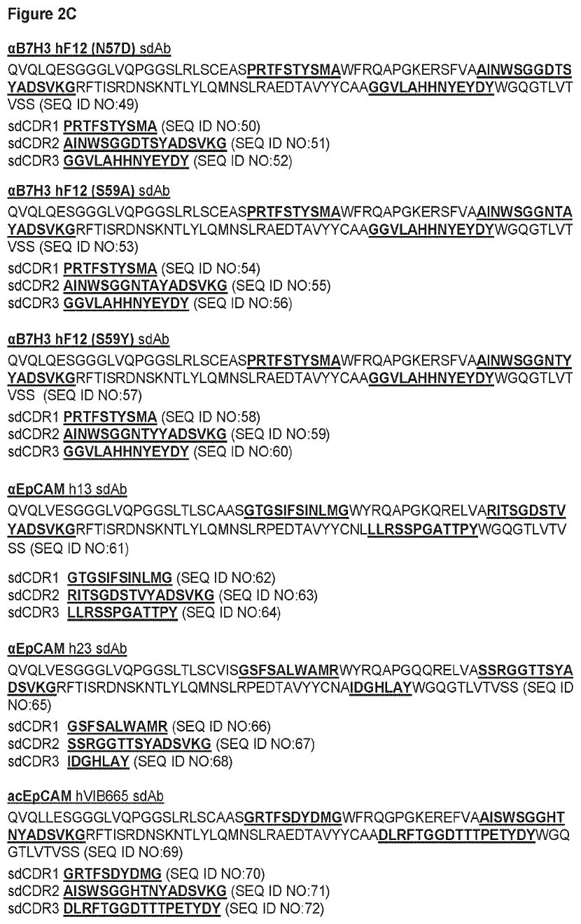

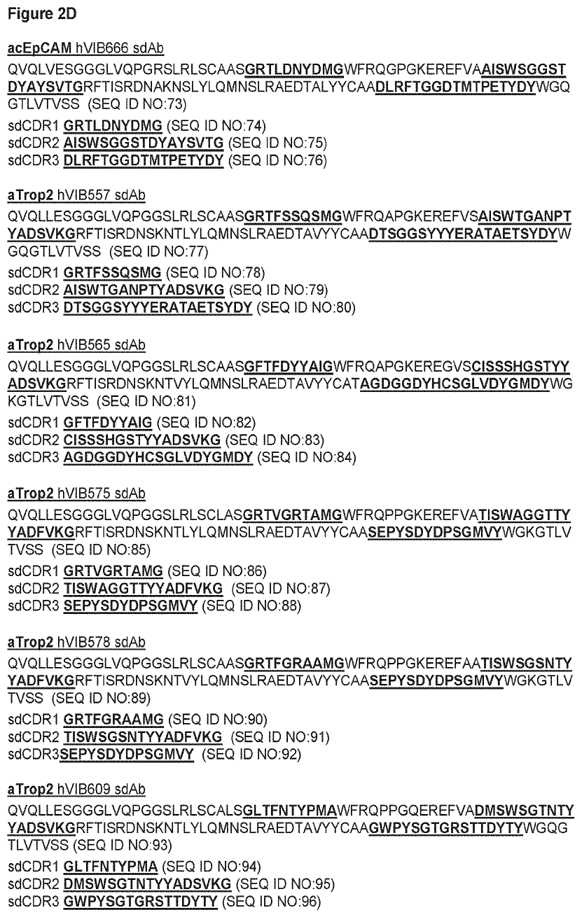

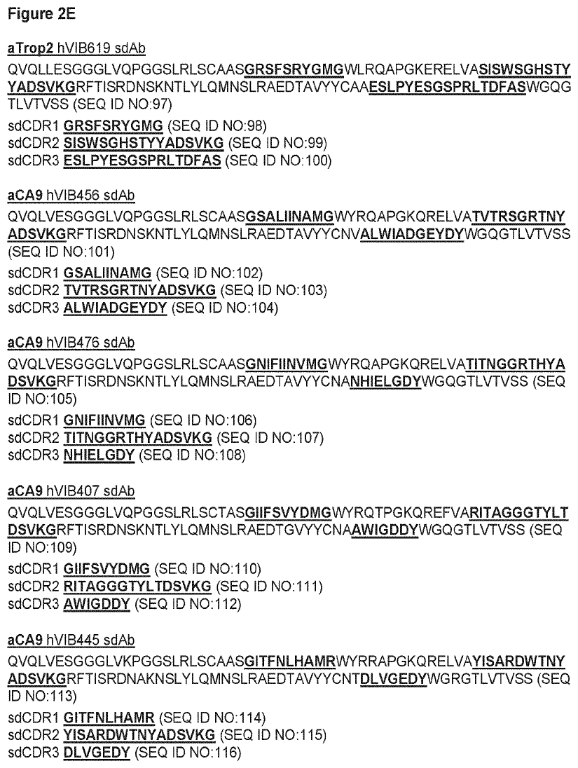

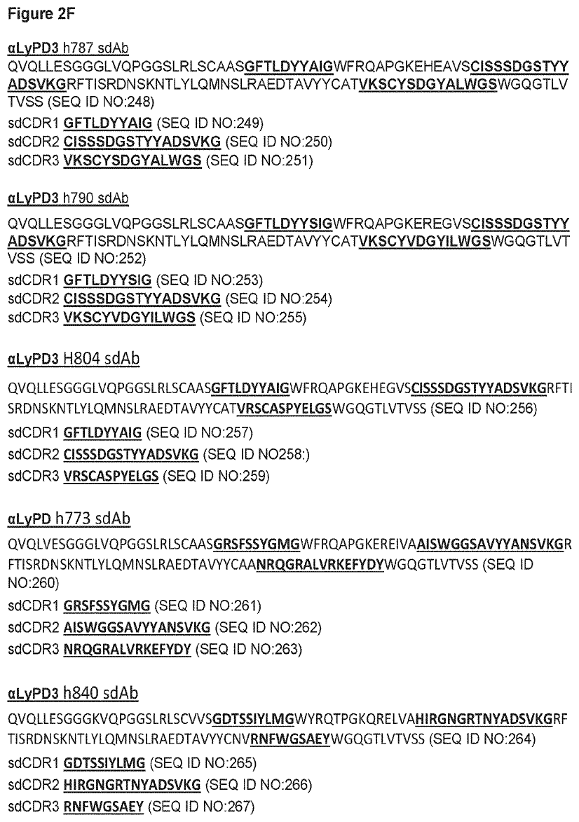

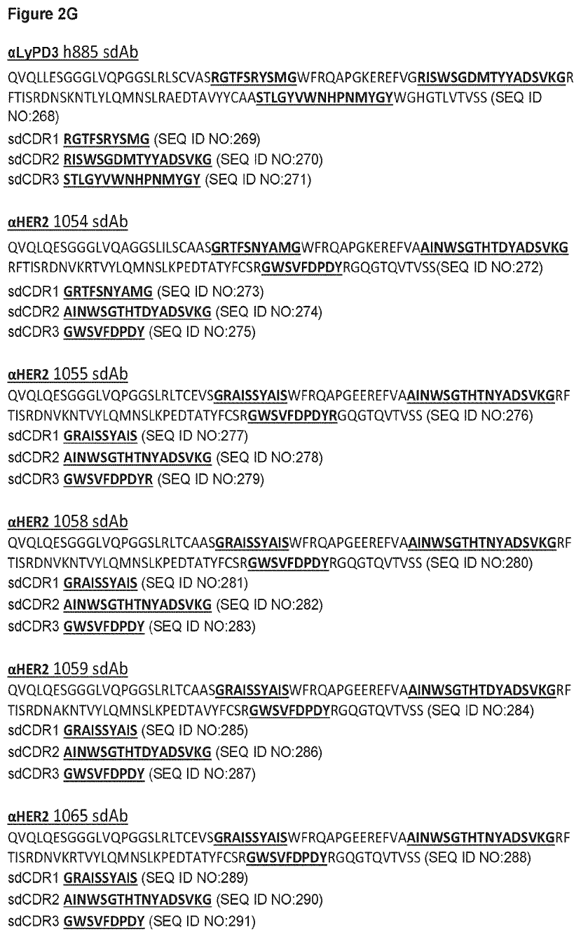

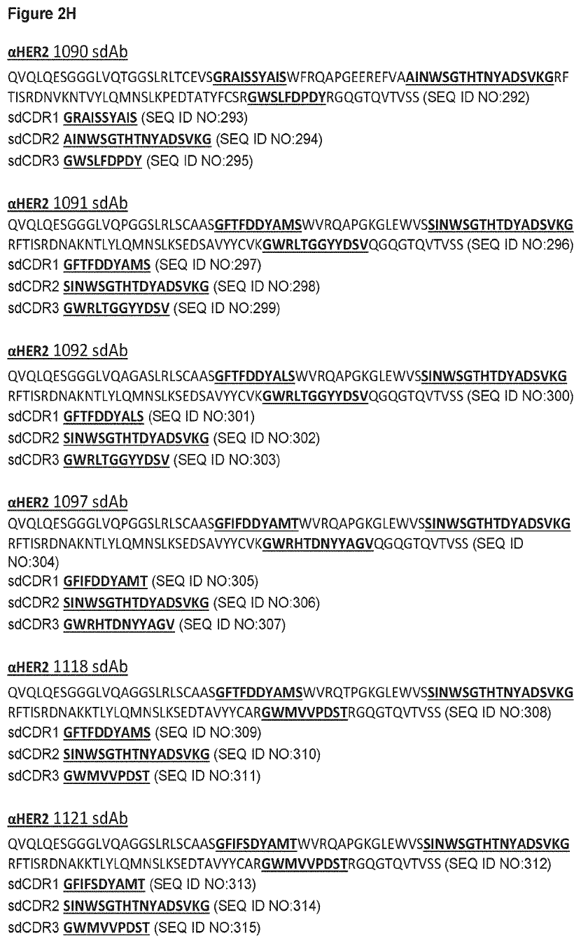

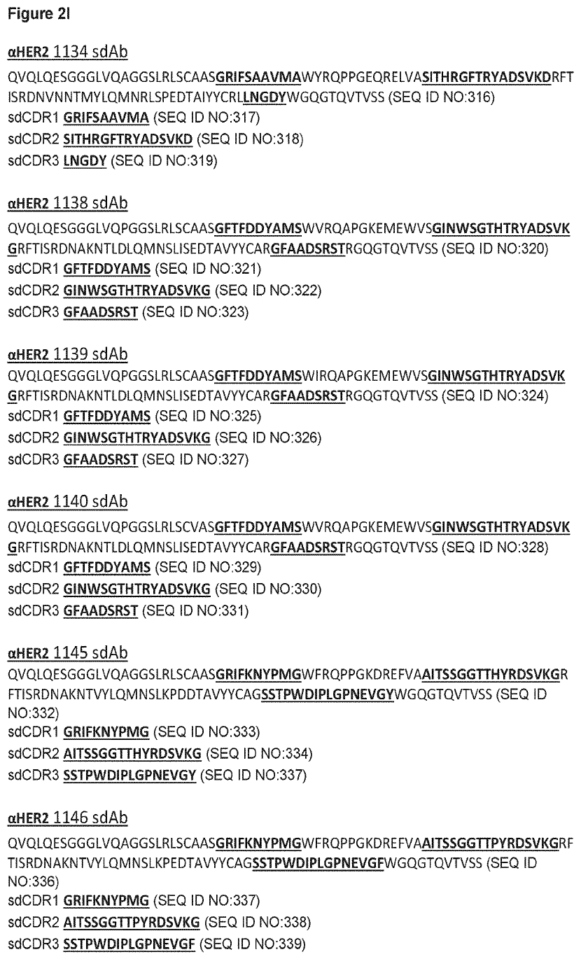

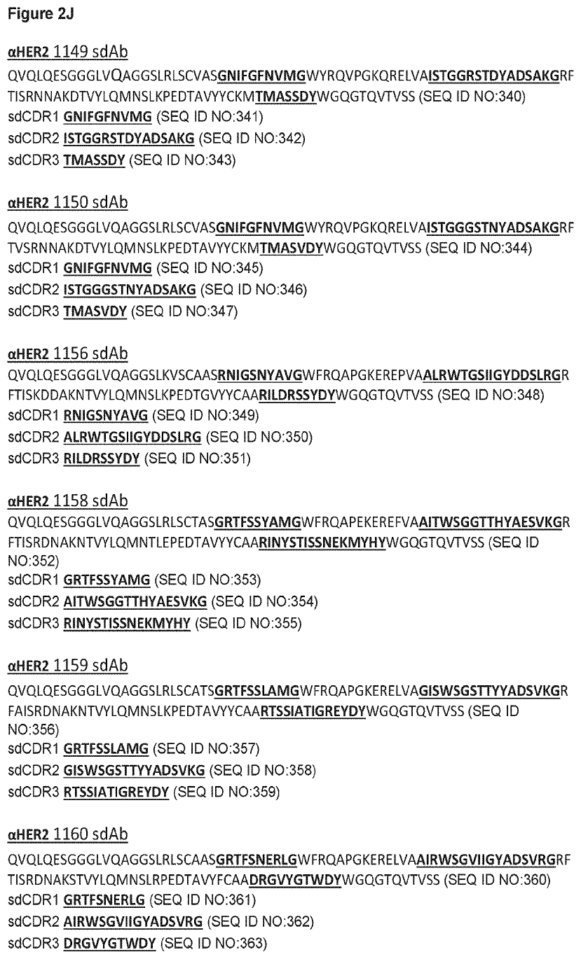

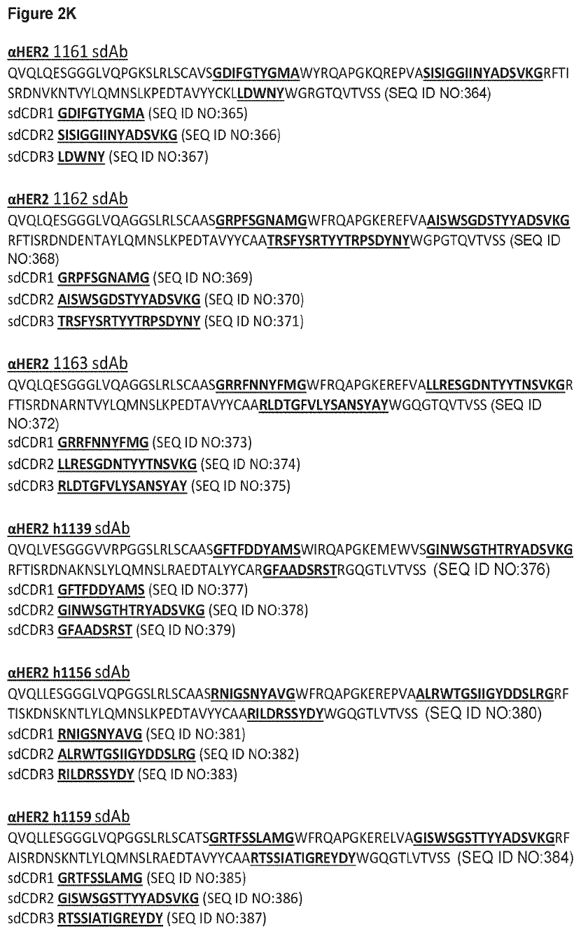

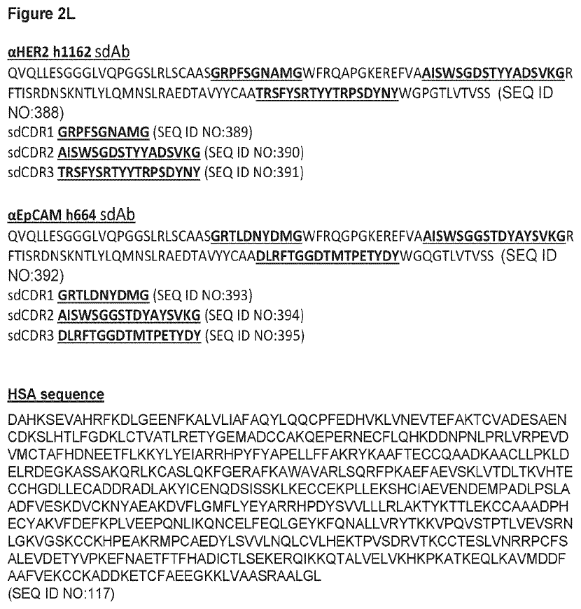

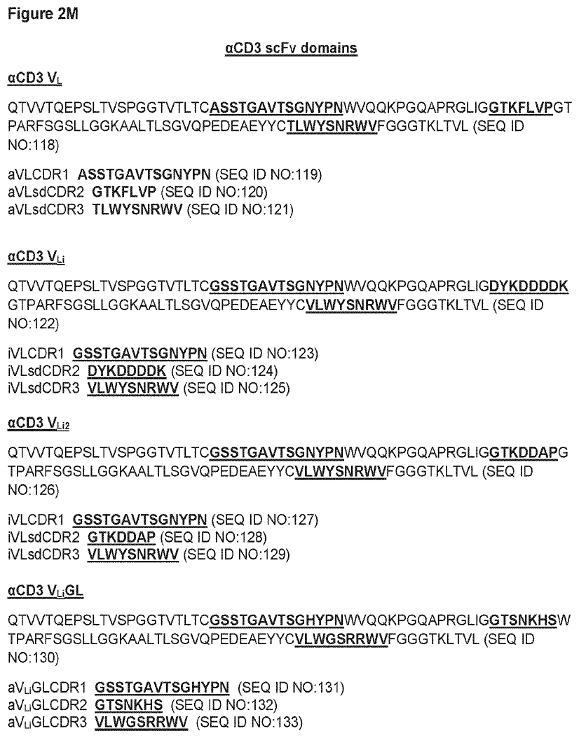

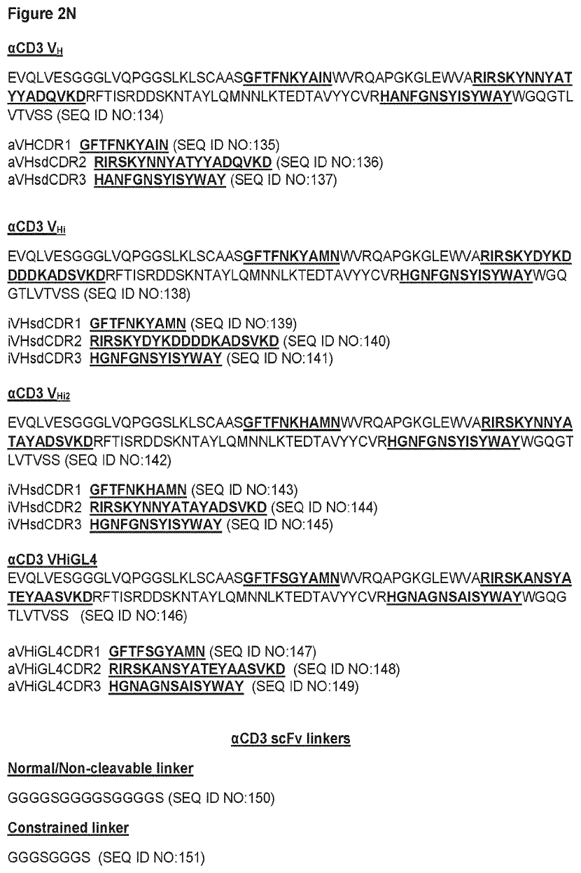

By “protease cleavage site” refers to the amino acid sequence recognized and cleaved by a protease. Suitable protease cleavage sites are outlined below and shown in FIGS. 2 and 3.

As used herein, “protease cleavage domain” refers to the peptide sequence incorporating the “protease cleavage site” and any linkers between individual protease cleavage sites and between the protease cleavage site(s) and the other functional components of the constructs of the invention (e.g., VH, VL, iVH, iVL, target antigen binding domain(s), HAS domain, etc.). As outlined herein, a protease cleavage domain may also include additional amino acids if necessary, for example to confer flexibility.

The term “COBRATM” and “conditional bispecific redirected activation” refers to a bispecific conditionally effective protein that has a number of functional protein domains. In some embodiments, one of the functional domains is an antigen binding domain (ABD) that binds a target tumor antigen (TTA). In certain embodiments, another domain is an ABD that binds to a T cell antigen under certain conditions. The T cell antigen includes but is not limited to CD3. The term “hemi-COBRA™” refers to a conditionally effective protein that can bind a T cell antigen when a variable heavy chain of a hemi-COBRA can associate to a variable light chain of another hemi-COBRATM (a complementary hemi-COBRA™) due to innate self-assembly when concentrated on the surface of a target expressing cell.

III. Fusion Proteins of the Invention

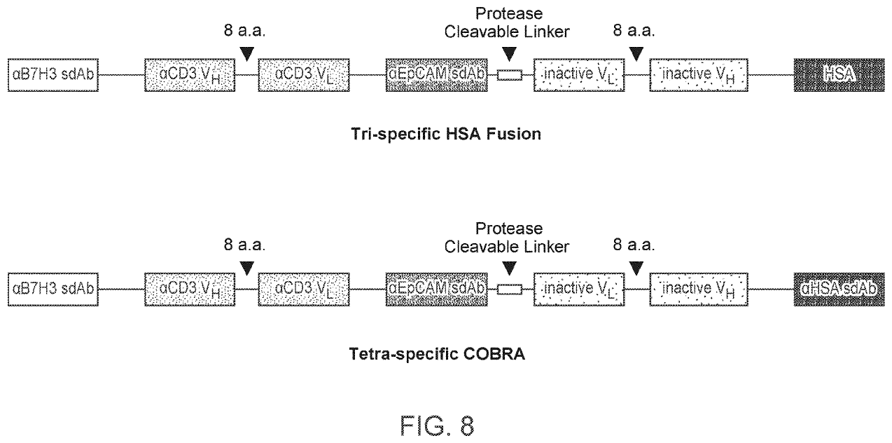

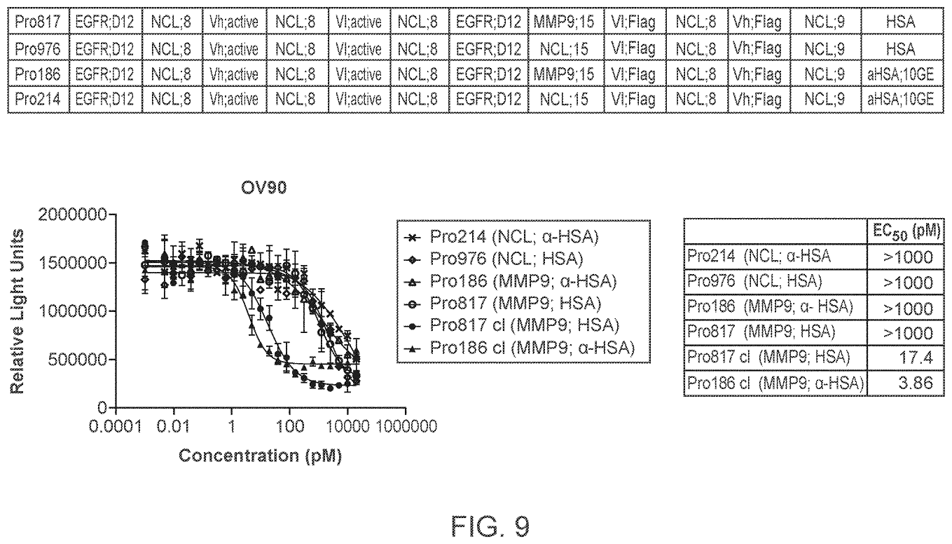

The fusion proteins of the invention have a number of different components, generally referred to herein as domains, that are linked together in a variety of ways. Some of the domains are binding domains, that each bind to a target antigen (e.g., a TTA or CD3, for example). As they bind to more than one antigen, they are referred to herein as “multispecific”; for example, a prodrug construct of the invention may bind to a TTA and CD3, and thus are “bispecific”. An exemplary schematic diagram of such a protein is found in FIGS. 1 and 8. A protein provided herein can also have higher specificities; for example, if the first αTTA binds to EGFR, the second αTTA binds to EpCAM and there is an anti-CD3 binding domain, this would be a “trispecific” molecule. Such fusion proteins are also referred to as prodrug proteins or hetero-specific fusion proteins. Similarly, the addition of an HSA binding domain to this construct would be “tetraspecific”; see FIG. 8.

As will be appreciated by those in the art, the proteins of the invention can have different valencies as well as be multispecific. That is, proteins of the invention can bind a target with more than one binding site; some constructs can have bivalent binding to a tumor antigen.

The prodrug proteins of the present technology can include CD3 antigen binding domains arranged in a variety of ways as outlined herein (see, for example, FIGS. 1 and 8), tumor target antigen binding domains, HSA domains, linkers (e.g.; protease cleavable linkers and non-cleavable linkers), etc. The prodrug proteins described herein have an increased (e.g., longer) serum half-life compared to corresponding proteins lacking an HSA domain. In some embodiments, the increased serum half-life is determined using a mouse surrogate for evaluating HSA pharmacokinetics such as aAlb^{-/-} hFcRn humanized mouse model (Tg32-Alb^{-/-} mFcRn^{-/-} hFcRn^{Tg/Tg} mouse model). In some embodiments, the serum half-life of any of the prodrug proteins described is increased by 5%, 10%, 20%, 30%, 40%, 50%, 60%, 70%, 80%, 90%, 100%, 105%, 110%, 120%; 130%, 140%, 150%; 160%, 170%, 180%, 190%, 200%, 250%, 300%, 350%, 400%, 450%, 500%, 550%, 600%, 650%, 700%, 450%, or more, compared to a similar protein without a half-life extension domain. In some embodiments, the serum half-life of any of the prodrug proteins is increased by 0.5 hr, 1.0 hr, 1.5 hrs, 2.0 hrs, 2.5 hrs, 3.0 hrs, 3.5 hrs, 4.0 hrs, 4.5 hrs, 5.0 hrs, 5.5 hrs, 6.0 hrs, 6.5 hrs, 7.0 hrs, 7.5 hrs, 8.0 hrs, 8.5 hrs, 9.0 hrs, 9.5 hrs, 10.0 hrs, 10.5 hrs, 11.0 hrs, 11.5 hrs, 12.0 hrs, 12.5 hrs, 13.0 hrs, 13.5 hrs, 14.0 hrs, 14.5 hrs, 15.0 hrs, 15.5 hrs, 16.0 hrs, 16.5 hrs, 17.0 hrs, 17.5 hrs, 18.0 hrs, 18.5 hrs, 19.0 hrs, 19.5 hrs, 20.0 hrs, 20,5 hrs, 21,0 hrs, 21.5 hrs, 22.0 hrs, 22.5 hrs, 23.0 hrs, 23.5 hrs, 24.0 hrs, 24.5 hrs, 25.0 hrs, 25.5 hrs, 26.0 hrs, 26.5 hrs, 27.0 hrs, 27.5 hrs, 28.0 hrs, 28.5 hrs, 29.0 hrs, 29.5 hrs, 30.0 hrs, 30.5 hrs, 31.0 hrs, 31.5 hrs, 32.0 hrs, or more; compared to a similar protein without a half-life extension domain. In some embodiments, the serum half-life of any of the prodrug proteins is increased by 0.5-fold, 1.0-fold, 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 9.5-fold, 10.0-fold, 10.5-fold, 11.0-fold, 11.5-fold, 12.0-fold, 12.5-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more, compared to a similar protein without a half-life extension domain.

In one aspect, the present invention provides fusion proteins comprising, from N- to C-terminal: a) a first single domain antigen binding domain (sdABD) that binds to a human tumor target antigen (TTA) (sdABD-TTA); b) a first domain linker; c) a constrained Fv domain comprising: i) a first variable heavy domain comprising a vhCDR1, vhCDR2 and vhCDR3; ii) a constrained non-cleavable linker (CNCL); and iii) a first variable light domain comprising v1CDR1, v1CDR2 and v1CDR3; d) a second domain linker; e) a second sdABD-TTA; f) a cleavable linker (CL); g) a constrained pseudo Fv domain comprising: i) a first pseudo light variable domain; ii) a non-cleavable linker (NCL); and iii) a first pseudo heavy variable domain; h) a third domain linker; and i) a human serum albumin (HSA) domain; wherein the first variable heavy domain and the first variable light domain are capable of binding human CD3 but the constrained Fv domain does not bind CD3; wherein the first variable heavy domain and the first pseudo variable light domain intramolecularly associate to form an inactive Fv; and wherein the first variable light domain and the first pseudo variable heavy domain intramolecularly associate to form an inactive Fv.

In some embodiments, the disclosure provides fusion proteins wherein the first variable heavy domain is N-terminal to the first variable light domain and the pseudo light variable domain is N-terminal to the pseudo variable heavy domain.

In some embodiments, the disclosure provides fusion proteins wherein the first variable heavy domain is N-terminal to the first variable light domain and the pseudo variable heavy domain is N-terminal to the pseudo variable light domain.

In some embodiments, the disclosure provides fusion proteins wherein the first variable light domain is N-terminal to the first variable heavy domain and the pseudo light variable domain is N-terminal to the pseudo variable heavy domain.

In some embodiments, the disclosure provides fusion proteins wherein the first variable light domain is N-terminal to the first variable heavy domain and the pseudo variable heavy domain is N-terminal to the pseudo variable light domain.

In some embodiments, the disclosure provides fusion proteins wherein the first and second TTA is the same.

In some embodiments, the disclosure provides fusion proteins wherein the first and second TTA are different.

In some embodiments, the disclosure provides fusion proteins wherein the first and second TTA are selected from EGFR, EpCAM, FOLR1, Trop2, CA9 (CAIX), B7H3, HER2, LyPD3, and any combination thereof. In some embodiments, the first TTA is EGFR, and the second TTA is selected from the group consisting of EpCAM, FOLR1, Trop2, CA9 (CAIX), HER2, LyPD3 and B7H3. In some embodiments, the first TTA is B7H3 and the second TTA is selected from the group consisting of EGFR, EpCAM, FOLR1, Trop2, CA9 (CAIX), LyPD3 and HER2. In some embodiments, the first TTA is HER2, and the second TTA is selected from the group consisting of EGFR, EpCAM, FOLR1, Trop2, CA9 (CAIX), LyPD3 and B7H3. In some embodiments, the first TTA is EpCAM and the second TTA is selected from the group consisting of EGFR, FOLR1, Trop2, CA9 (CAIX), LyPD3, HER2 and B7H3. In some embodiments, the first TTA is FOLR1 and the second TTA is selected from the group consisting of EGFR, Trop2, CA9 (CAIX), HER2, EpCAM, LyPD3 and B7H3. In some embodiments, the first TTA is CA9 (CAIX) and the second TTA is selected from the group consisting of EGFR, Trop2, FOLR1, HER2, EpCAM, LyPD3 and B7H3. In some embodiments, the first TTA is Trop2 and the second TTA is selected from the group consisting of EGFR, CA9 (CAIX), FOLR1, HER2, EpCAM, LyPD3 and B7H3. In some embodiments, the first TTA is LyPD3 and the second TTA is selected from the group consisting of EGFR, CA9 (CAIX), FOLR1, HER2, EpCAM, Trop2 and B7H3.

In some embodiments, the disclosure provides fusion proteins wherein the first and/or second sdABD-TTAs are selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, SEQ ID NO:344, and any combination thereof. In some instances, the first sdABD-TTA is selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, and SEQ ID NO:344. In some instances, the second sdABD-TTA is selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, and SEQ ID NO:344.

In some embodiments, the disclosure provides fusion proteins wherein the HSA domain has SEQ ID NO:117.

In some embodiments, the disclosure provides fusion proteins wherein the cleavable linker is cleaved by a human protease selected from the group consisting of MMP2, MMP9, meprin A, meprin B, cathepsin S, cathepsin K, cathespin L, granzymeB, uPA, kallekriein7, matriptase and thrombin.

In some embodiments, the disclosure provides nucleic acids that encode a fusion protein of the invention, expression vectors comprising the expression vectors, and host cells comprising the nucleic acids or expression vectors.

In some embodiments, the disclosure provides methods of making the fusion proteins of the invention comprising culturing the host cells under conditions wherein the protein is expressed and recovering the protein.

In some embodiments, the disclosure provides methods of treating cancer comprising administering a fusion protein of the invention to a patient.
A. CD3 Antigen Binding Domains

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, two CD3e (epsilon) chains and two CD3ζ (zeta) chains, which are present at the cell surface. CD3 molecules associate with the a (alpha) and β (beta) chains of the T cell receptor (TCR) to comprise the TCR complex. Clustering of CD3 on T cells, such as by Fv domains that bind to CD3 leads to T cell activation similar to the engagement of the T cell receptor but independent of its clonal-typical specificity.

However, as is known in the art, CD3 activation can cause a number of toxic side effects, and accordingly the present invention is directed to providing active CD3 binding of the polypeptides of the invention only in the presence of tumor cells, where specific proteases are found, that then cleave the prodrug polypeptides of the invention to provide an active CD3 binding domain. Thus, in the present invention, binding of an anti-CD3 Fv domain to CD3 is regulated by a protease cleavage domain which restricts binding of the CD3 Fv domain to CD3, only in the microenvironment of a diseased cell or tissue with elevated levels of proteases, for example in a tumor microenvironment as is described herein.

Accordingly, the present invention provides two sets of VH and VL domains, an active set (VH and VL) and an inactive set (iVH and iVL) with all four being present in the prodrug construct. The construct is formatted such that the VH and VL set cannot self-associate, but rather associate with their inactive partners, e.g., iVH and VL and iVL and VH as is shown herein.

1. Active anti-CD3 variable heavy and variable light domains

There are a number of suitable active CDR sets, and/or VH and VL domains, that are known in the art that find use in the present invention. For example, the CDRs and/or VH and VL domains are derived from known anti-CD3 antibodies, such as, for example, muromonab-CD3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the VH and VL sequences that form an active Fv domain that binds to human CD3 are shown in FIGS. 2M and 2N. As is shown herein, these active VH (“aVH”) and active VL (“aVL”) domains can be used in different configurations as described herein.

2. Inactive anti-CD3 variable heavy and variable light domains

The inactive iVH and iVL domains contain “regular” framework regions (FRs) that allow association, such that an inactive variable domain will associate with an active variable domain, rendering the pair inactive, e.g., unable to bind CD3.

As will be appreciated by those in the art, there are a number of “inactive” variable domains that find use in the invention. Basically, any variable domain with human framework regions that allows self-assembly with another variable domain, no matter what amino acids are in the CDR location in the variable region, can be used. For clarity, the inactive domains are the to include CDRs, although technically the inactive variable domains do not confer binding capabilities.

As will be appreciated in the art, it is generally straightforward to generate inactive VH or VL domains, and can be done in a variety of ways. In some embodiments, the generation of inactive variable domains is generally done by altering one or more of the CDRs of an active Fv, including making changes in one or more of the three CDRs of an active variable domain. This can be done by making one or more amino acid substitutions at functionally important residues in one or more CDRs, replacing some or all CDR residues with random sequences, replacing one or more CDRs with tag or flag sequences, and/or swapping CDRs and/or variable regions with those from an irrelevant antibody (one directed to a different organism's protein for example.

In some cases, only one of the CDRs in a variable region can be altered to render it inactive, although other embodiments include alterations in one, two, three, four, five or six CDRs.

In some cases, the inactive domains can be engineered to promote selective binding in the prodrug format, to encourage formation of intramolecular iVH-VL and VH-iVL domains prior to cleavage (over, for example, intermolecular pair formation). See for example, Igawa et al., Protein Eng. Des. Selection, 2010, 23(8):667-677, hereby expressly incorporated by reference in its entirety and specifically for the interface residue amino acid substitutions.

In certain embodiments, the CD3 binding domain of the polypeptide constructs described herein exhibit not only potent CD3 binding affinities with human CD3, but show also excellent cross reactivity with the respective cynomolgus monkey CD3 proteins. In some instances, the CD3 binding domain of the polypeptide constructs is cross-reactive with CD3 from cynomolgus monkey. In certain instances, human:cynomolgous KD ratios for CD3 are between 5 and 0.2.

In some embodiments, the CD3 binding domain of the antigen binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the antigen binding protein will ultimately be used in. For example, for use in humans, it may be beneficial for the CD3 binding domain of the antigen binding protein to comprise human or humanized residues from the antigen binding domain of an antibody or antibody fragment.

Thus, in one aspect, the antigen-binding domain comprises a humanized or human binding domain. In one embodiment, the humanized or human anti-CD3 binding domain comprises one or more (e.g., all three) light chain complementary determining region 1 (LC CDR1 or v1CDR1), light chain complementary determining region 2 (LC CDR2 or v1CDR2), and light chain complementary determining region 3 (LC CDR3 or v1CDR3) of a humanized or human anti-CD3 binding domain described herein, and/or one or more (e.g., all three) heavy chain complementary determining region 1 (HC CDR1 or vhCDR1), heavy chain complementary determining region 2 (HC CDR2 or vhCDR2), and heavy chain complementary determining region 3 (HC CDR3 or vhCDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, one or more CD3 binding domains are humanized or fully human. In some embodiments, one or more activated CD3 binding domains have a KD binding of 1000 nM or less to CD3 on CD3 expressing cells. In some embodiments, one or more activated CD3 binding domains have a KD binding of 100 nM or less to CD3 on CD3 expressing cells. In some embodiments, one or more activated CD3 binding domains have a KD binding of 10 nM or less to CD3 on CD3 expressing cells. In some embodiments, one or more CD3 binding domains have cross-reactivity with cynomolgus CD3. In some embodiments, one or more CD3 binding domains comprise an amino acid sequence provided herein.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In one embodiment, the anti-CD3 binding domain is an Fv comprising a light chain and a heavy chain of an amino acid sequence provided herein. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region- scFv linker-heavy chain variable region or heavy chain variable region- scFv linker-light chain variable region.

In some embodiments, CD3 binding domain of an antigen binding protein has an affinity to CD3 on CD3 expressing cells with a KD of 1000 nM or less, 100 nM or less, 50 nM or less, 20 nM or less, 10 nM or less, 5 nM or less, 1 nM or less, or 0.5 nM or less. In some embodiments, the CD3 binding domain of an antigen binding protein has an affinity to CD3ε with a KD of 1000 nM or less, 100 nM or less, 50 nM or less, 20 nM or less, 10 nM or less, 5 nM or less, 1 nM or less, or 0.5 nM or less. In further embodiments, CD3 binding domain of an antigen binding protein has low affinity to CD3, i.e., about 100 nM or greater.

The affinity to bind to CD3 can be determined, for example, by the ability of the antigen binding protein itself or its CD3 binding domain to bind to CD3 coated on an assay plate; displayed on a microbial cell surface; in solution; etc., as is known in the art, generally using Biacore or Octet assays. The binding activity of the antigen binding protein itself or its CD3 binding domain of the present disclosure to CD3 can be assayed by immobilizing the ligand (e.g., CD3) or the antigen binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

In many embodiments, active and inactive (inert) binding domains are those shown in FIGS. 2M and 2N.
B. Antigen Binding Domains to Tumor Target Antigens (TTAs)

In addition to the described anti-CD3 and HSA domains, the polypeptide constructs described herein also comprise target domains that bind to one or more target antigens or one or more regions on a single target antigen. It is contemplated herein that a polypeptide construct of the invention is cleaved, for example, in a disease-specific microenvironment or in the blood of a subject at the protease cleavage domain and that each target antigen binding domain will bind to a target antigen on a target cell, thereby activating the CD3 binding domain to bind a T cell. In general, the TTA binding domains can bind to their targets before protease cleavage, so they can “wait” on the target cell to be activated as T cell engagers. At least one target antigen is involved in and/or associated with a disease, disorder or condition. Exemplary target antigens include those associated with a proliferative disease, a tumorous disease, an inflammatory disease, an immunological disorder; an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium. At least one target antigen may also be directed against healthy tissue.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

Preferred embodiments of the invention utilize sdABDs as the TTA binding domains. These are preferred over scFv ABDs, since the addition of other VH and VL domains into a construct of the invention may complicate the formation of pseudo Fv domains.

In some embodiments the pro-drug constructs of the invention utilize two TTA ABDs, again preferably in the sdABD-TTA format. When dual targeting domains are used, they can bind to the same epitope of the same TTA. For example, as discussed herein, many of the constructs herein utilize two identical targeting domains. In some embodiments, two targeting domains can be used that bind to different epitopes of the same TTA, for example as shown in FIG. 1, the two EGFR sdABDs bind to different epitopes on human EGFR. In some instances, a pro-drug construct described herein with dual targeting domains that bind the same TTA is referred to as a mono-specific COBRA construct. In some embodiments, the two targeting domains bind to different TTAs, see for example FIG. 8. In some embodiments of a dual targeting pro-drug construct, a first targeting domain binds to a TTA such as B7H3, CA9, EGFR, EpCAM, FOLR1, HER2, LyPD3, or Trop2, and a second targeting domain binds to a different TTA selected from the group consisting of B7H3, CA9, EGFR, EpCAM, FOLR1, HER2, LyPD3, and Trop2. In some instances, a pro-drug construct described herein with dual targeting domains that bind different TTAs is referred to as a hetero-specific COBRA construct.

Polypeptide constructs contemplated herein include at least one antigen binding domain, wherein the antigen binding domain binds to at least one target antigen, e.g., a tumor target antigen. In some embodiments, the target antigen binding domains specifically bind to a cell surface molecule. In some embodiments, the target antigen binding domains specifically bind to a tumor antigen. In some embodiments, the target antigen binding domains specifically and independently bind to a tumor target antigen (TTA) selected from at least one of EpCAM, EGFR, HER2, HER3, cMet, LyPD3, B7H3, Trop2, CA9, CEA, and FOLR1. In some embodiments, the TTA is selected from the group consisting of B7H3, CA9, EGFR, EpCAM, FOLR1, HER2, LyPD3, and Trop2.
a) B7H3 sdABDs

Further embodiments of use in the invention are sdABDs to human B7H3 as shown in FIGS. 2B-2C. In some embodiments, the sdABD-B7H3 (e.g., sdABD-B7H3 hF7) has a sdCDR1 with SEQ ID NO:34, a sdCDR2 with SEQ ID NO:35, and a sdCDR3 with SEQ ID NO:36. In some cases, the sdABD-B7H3 has the amino acid sequence of SEQ ID NO:33, as provided as FIG. 2B. In some embodiments, the sdABD-B7H3 (e.g., sdABD-B7H3 hF12) has a sdCDR1 with SEQ ID NO:38, a sdCDR2 with SEQ ID NO:39, and a sdCDR3 with SEQ ID NO:40. In some cases, the sdABD-B7H3 has the amino acid sequence of SEQ ID NO:37, as provided as FIG. 2B. In some embodiments, the sdABD-B7H3 (e.g., sdABD-B7H3 hF12 (N57Q)) has a sdCDR1 with SEQ ID NO:42, a sdCDR2 with SEQ ID NO:43, and a sdCDR3 with SEQ ID NO:44. In some cases, the sdABD-B7H3 has the amino acid sequence of SEQ ID NO:41, as provided as FIG. 2B. In contrast to the hF7 and hF12 B7H3 sdABDs, the amino acid substitution N57Q removes a glycosylation site. In some embodiments, the sdABD-B7H3 (e.g., sdABD-B7H3 HF12 (N57E)) has a sdCDR1 with SEQ ID NO:46, a sdCDR2 with SEQ ID NO:47, and a sdCDR3 with SEQ ID NO:48. In some cases, the sdABD-B7H3 has the amino acid sequence of SEQ ID NO:45, as provided as FIG. 2B. In contrast to the hF7 and hF12 B7H3 sdABDs, the amino acid substitution N57E removes a glycosylation site. In some embodiments, the sdABD-B7H3 (e.g., sdABD-B7H3 hF12 (N57D)) has a sdCDR1 with SEQ ID NO:50, a sdCDR2 with SEQ ID NO:51, and a sdCDR3 with SEQ ID NO:52. In some cases, the sdABD-B7H3 has the amino acid sequence of SEQ ID NO:49, as provided as FIG. 2C. In contrast to the hF7 and hF12 B7H3 sdABDs, the amino acid substitution N57D removes a glycosylation site. In some embodiments, the sdABD-B7H3 (e.g., sdABD-B7H3 hF12(S59A)) has a sdCDR1 with SEQ ID NO:54, a sdCDR2 with SEQ ID NO:55, and a sdCDR3 with SEQ ID NO:56. In some cases, the sdABD-B7H3 has the amino acid sequence of SEQ ID NO:53, as provided as FIG. 2C. In contrast to the hF7 and hF12 B7H3 sdABDs, the amino acid substitution S59A removes a glycosylation site. In some embodiments, the sdABD-B7H3 (e.g., sdABD-B7H3 hF12 (S59Y)) has a sdCDR1 with SEQ ID NO:58, a sdCDR2 with SEQ ID NO:59, and a sdCDR3 with SEQ ID NO:60. In some cases, the sdABD-B7H3 has the amino acid sequence of SEQ ID NO:57, as provided as FIG. 2C. In contrast to the hF7 and hF12 B7H3 sdABDs, the amino acid substitution NS59Y removes a glycosylation site.
b) CA9 (CAIX) sdABDs

Additional embodiments of use in the invention are sdABDs to human CA9 as shown in FIG. 2E. In some embodiments, the sdABD-CA9 (e.g., sdABD-CA9 hVIB456) has a sdCDR1 with SEQ ID NO:102, a sdCDR2 with SEQ ID NO:103, a sdCDR3 with SEQ ID NO:104. In some cases, the sdABD-Trop2 has the amino acid sequence of SEQ ID NO:101, as provided in FIG. 2E. In some embodiments, the sdABD-CA9 (e.g., sdABD-CA9 hVIB476) has a sdCDR1 with SEQ ID NO:106, a sdCDR2 with SEQ ID NO:107, and a sdCDR3 with SEQ ID NO:108. In some cases, the sdABD-Trop2 has the amino acid sequence of SEQ ID NO:105, as provided in FIG. 2E. In some embodiments, the sdABD-CA9 (e.g., sdABD-CA9 hVIB407) has a sdCDR1 with SEQ ID NO:110, a sdCDR2 with SEQ ID NO:111, and a sdCDR3 with SEQ ID NO:112. In some cases, the sdABD-Trop2 has the amino acid sequence of SEQ ID NO:109, as provided in FIG. 2E. In some embodiments, the sdABD-CA9 (e.g., sdABD-CA9 hVIB445) has a sdCDR1 with SEQ ID NO:114, a sdCDR2 with SEQ ID NO:115, and a sdCDR3 with SEQ ID NO:116. In some cases, the sdABD-Trop2 has the amino acid sequence of SEQ ID NO:113, as provided in FIG. 2E.
c) EGFR sdABDs

Of particular use in the present invention are sdABDs to human EGFR as shown in FIG. 2. In some embodiments, the sdABD-EGFR (e.g., sdABD-αEGFR1) has a sdCDR1 with SEQ ID NO:2 a sdCDR2 with SEQ ID NO:3 and a sdCDR3 with SEQ ID NO:4. In some cases, the sdABD-EGFR has the amino acid sequence of SEQ ID NO:1, as provided in FIG. 2A. In some embodiments, the sdABD-EGFR (e.g., sdABD-αEGFR2) has a sdCDR1 with SEQ ID NO:6, a sdCDR2 with SEQ ID NO:7 and a sdCDR3 with SEQ ID NO:8. In some cases, the sdABD-EGFR has the amino acid sequence of SEQ ID NO:5, as provided in FIG. 2A. In some embodiments, the sdABD-EGFR (e.g., sdABD-hαEGFR1) has a sdCDR1 with SEQ ID NO:10, a sdCDR2 with SEQ ID NO:11 and a sdCDR3 with SEQ ID NO:12. In some cases, the sdABD-EGFR has the amino acid sequence of SEQ ID NO:9, as provided in FIG. 2A. In some embodiments, the sdABD-EGFR (e.g., sdABD-aEGFR2a) has a sdCDR1 with SEQ ID NO:14, a sdCDR2 with SEQ ID NO:15 and a sdCDR3 with SEQ ID NO:16. In some cases, the sdABD-EGFR has the amino acid sequence of SEQ ID NO:13, as provided in FIG. 2A. In some embodiments, the sdABD-EGFR (e.g., sdABD-hαEGFR2d) has a sdCDR1 with SEQ ID NO:18, a sdCDR2 with SEQ ID NO:19 and a sdCDR3 with SEQ ID NO:20. In some cases, the sdABD-EGFR has the amino acid sequence of SEQ ID NO:17, as provided in FIG. 2A.
a) EpCAM sdABDs

Additional embodiments of use in the invention are sdABDs to human EpCAM as shown in FIGS. 2C, 2D and 2L. In some embodiments, the sdABD-EpCAM (e.g., sdABD-EpCAM h13) has a sdCDR1 with SEQ ID NO:62, a sdCDR2 with SEQ ID NO:63, and a sdCDR3 with SEQ ID NO:64. In some cases, the sdABD-EpCAM has the amino acid sequence of SEQ ID NO:61, as provided in FIG. 2C. In some embodiments, the sdABD-EpCAM (e.g., sdABD-EpCAM h23) has a sdCDR1 with SEQ ID NO:66, a sdCDR2 with SEQ ID NO:67, and a sdCDR3 with SEQ ID NO:68. In some cases, the sdABD-EpCAM has the amino acid sequence of SEQ ID NO:65, as provided in FIG. 2C. In some embodiments, the sdABD-EpCAM (e.g., sdABD-EpCAM hVIB665) has a sdCDR1 with SEQ ID NO:70, a sdCDR2 with SEQ ID NO:71, and a sdCDR3 with SEQ ID NO:72. In some cases, the sdABD-EpCAM has the amino acid sequence of SEQ ID NO:69, as provided in FIG. 2C. It should be noted that in contrast to the h13 and h23 EpCAM sdABDs, hVIB665 (also referred to as “acEpCAM hVIB665”) binds to both the cleaved and uncleaved form of EpCAM (which is known to undergo a cleavage in vivo). In some embodiments, the sdABD-EpCAM (e.g., sdABD-EpCAM hVIB666) has a sdCDR1 with SEQ ID NO:74, a sdCDR2 with SEQ ID NO:75, and a sdCDR3 with SEQ ID NO:76. In some cases, the sdABD-EpCAM has the amino acid sequence of SEQ ID NO:73, as provided in FIG. 2D. It should be noted that in contrast to the h13 and h23 EpCAM sdABDs, hVIB666 (also referred to as “acEpCAM hVIB666”) binds to both the cleaved and uncleaved form of EpCAM (which is known to undergo a cleavage in vivo). In some embodiments, the sdABD-EpCAM (e.g., humanized a EpCAM sdAb) has a sdCDR1 with SEQ ID NO:393, a sdCDR2 with SEQ ID NO:394, and a sdCDR3 with SEQ ID NO:395. In some cases, the sdABD-EpCAM has the amino acid sequence of SEQ ID NO:392, as provided in FIG. 2L.
b) FOLR1 sdABDs

Additional embodiments of use in the invention are sdABDs to human FOLR1 as shown in FIGS. 2A-2B. In some embodiments, the sdABD-FOLR1 (e.g., sdABD-FOLR1 h77-2) has a sdCDR1 with SEQ ID NO:22, a sdCDR2 with SEQ ID NO:23, and a sdCDR3 with SEQ ID NO:24. In some cases, the sdABD-FOLR1 has the amino acid sequence of SEQ ID NO:21, as provided in FIG. 2A. In some embodiments, the sdABD-FOLR1 (e.g., sdABD-FOLR1 h59.3) has a sdCDR1 with SEQ ID NO:26, a sdCDR2 with SEQ ID NO:27, and a sdCDR3 with SEQ ID NO:28. In some cases, the sdABD-FOLR1 has the amino acid sequence of SEQ ID NO:25, as provided in FIG. 2B. In some embodiments, the sdABD-FOLR1 (e.g., sdABD-FOLR1 h22-4) has a sdCDR1 with SEQ ID NO:30, a sdCDR2 with SEQ ID NO:31, and a sdCDR3 with SEQ ID NO:32. In some cases, the sdABD-FOLR1 has the amino acid sequence of SEQ ID NO:29, as provided in FIG. 2B.
c) HER2 sdABDs

Additional embodiments of use in the invention are sdABDs to human HER2 as shown in FIGS. 2G-2L. In some embodiments, the sdABD-HER2 (e.g., sdABD-HER2 1054) has a sdCDR1 with SEQ ID NO:273, a sdCDR2 with SEQ ID NO:274, and a sdCDR3 with SEQ ID NO:275. In some cases, the sdABD-HER2 has the amino acid sequence of SEQ ID NO:272, as provided in FIG. 2G. In some embodiments, the sdABD-HER2 (e.g., sdABD-HER2 1055) has a sdCDR1 with SEQ ID NO:277, a sdCDR2 with SEQ ID NO:278, and a sdCDR3 with SEQ ID NO:279. In some cases, the sdABD-HER2 has the amino acid sequence of SEQ ID NO:276, as provided in FIG. 2G. In some embodiments, the sdABD-HER2 (e.g., sdABD-HER2 1058) has a sdCDR1 with SEQ ID NO:281, a sdCDR2 with SEQ ID NO:282, and a sdCDR3 with SEQ ID NO:283. In some cases, the sdABD-HER2 has the amino acid sequence of SEQ ID NO:280, as provided in FIG. 2G. In some embodiments, the sdABD-HER2 (e.g., sdABD-HER2 1059) has a sdCDR1 with SEQ ID NO:285, a sdCDR2 with SEQ ID NO:286, and a sdCDR3 with SEQ ID NO:287. In some cases, the sdABD-HER2 has the amino acid sequence of SEQ ID NO:284, as provided in FIG. 2G. some embodiments, the sdABD-HER2 (e.g., sdABD-HER2 1065) has a sdCDR1 with SEQ ID NO:289, a sdCDR2 with SEQ ID NO:290, and a sdCDR3 with SEQ ID NO:291. In some cases, the sdABD-HER2 has the amino acid sequence of SEQ ID NO:288, as provided in FIG. 2G. In some embodiments, the sdABD-HER2 (e.g., sdABD-HER2 1090) has a sdCDR1 with SEQ ID NO:293, a sdCDR2 with SEQ ID NO:294, a sdCDR3 with SEQ ID NO:295. In some cases, the sdABD-HER2 has the amino acid sequence of SEQ ID NO:292, as provided in FIG. 2H. In some embodiments, the sdABD-HER2 (e.g., sdABD-HER2 1091) has a sdCDR1 with SEQ ID NO:297, a sdCDR2 with SEQ ID NO:298, and a sdCDR3 with SEQ ID NO:299. In some cases, the sdABD-HER2 has the amino acid sequence of SEQ ID NO:296, as provided in FIG. 2H. In some embodiments, the sdABD-HER2 (e.g., sdABD-HER2 1092) has a sdCDR1 with SEQ ID NO:301, a sdCDR2 with SEQ ID NO:302, and a sdCDR3 with SEQ ID NO:303. In some cases, the sdABD-HER2 has the amino acid sequence of SEQ ID NO:300, as provided in FIG. 2H. In some embodiments, the sdABD-HER2 (e.g., sdABD-HER2 1097) has a sdCDR1 with SEQ ID NO:305, a sdCDR2 with SEQ ID NO:306, and a sdCDR3 with SEQ ID NO:307. In some cases, the sdABD-HER2 has the amino acid sequence of SEQ ID NO:304, as provided in FIG. 2H. In some embodiments, the sdABD-HER2 (e.g., sdABD-HER2 1118) has a sdCDR1 with SEQ ID NO:309, a sdCDR2 with SEQ ID NO:310, and a sdCDR3 with SEQ ID NO:311. In some cases, the sdABD-HER2 has the amino acid sequence of SEQ ID NO:308, as provided in FIG. 2H. In some embodiments, the sdABD-HER2 (e.g., sdABD-HER2 1121) has a sdCDR1 with SEQ ID NO:313, a sdCDR2 with SEQ ID NO:314, and a sdCDR3 with SEQ ID NO:315, In some cases, the sdABD-HER2 has the amino acid sequence of SEQ ID NO:312, as provided in FIG. 2H. In some embodiments, the sdABD-HER2 (e.g., sdABD-HER2 1134) has a sdCDR1 with SEQ ID NO:317, a sdCDR2 with SEQ ID NO:318, and a sdCDR3 with SEQ ID NO:319. In some cases, the sdABD-HER2 has the amino acid sequence of SEQ ID NO:316, as provided in FIG. 2I. In some embodiments, the sdABD-HER2 (e.g., sdABD-HER2 1138) has a sdCDR1 with SEQ ID NO:321, a sdCDR2 with SEQ ID NO:322, and a sdCDR3 with SEQ ID NO:323. In some cases, the sdABD-HER2 has the amino acid sequence of SEQ ID NO:320, as provided in FIG. 2I. In some embodiments, the sdABD-HER2 (e.g., sdABD-HER2 1139) has a sdCDR1 with SEQ ID NO:325, a sdCDR2 with SEQ ID NO:326, and a sdCDR3 with SEQ ID NO:327. In some cases, the sdABD-HER2 has the amino acid sequence of SEQ ID NO:324, as provided in FIG. 2I. In some embodiments, the sdABD-HER2 (e.g., sdABD-HER2 1140) has a sdCDR1 with SEQ ID NO:329, a sdCDR2 with SEQ ID NO:330, and a sdCDR3 with SEQ ID NO:331. In some cases, the sdABD-HER2 has the amino acid sequence of SEQ ID NO:328, as provided in FIG. 2I. In some embodiments, the sdABD-HER2 (e.g., sdABD-HER2 1145) has a sdCDR1 with SEQ ID NO:333, a sdCDR2 with SEQ ID NO:334, and a sdCDR3 with SEQ ID NO:335. In some cases, the sdABD-HER2 has the amino acid sequence of SEQ ID NO:332, as provided in FIG. 2I. In some embodiments, the sdABD-HER2 (e.g., sdABD-HER2 1146) has a sdCDR1 with SEQ ID NO:337, a sdCDR2 with SEQ ID NO:338, and a sdCDR3 with SEQ ID NO:339. In some cases, the sdABD-HER2 has the amino acid sequence of SEQ ID NO:336, as provided in FIG. 2I. In some embodiments, the sdABD-HER2 (e.g., sdABD-HER2 1149) has a sdCDR1 with SEQ ID NO:341, a sdCDR2 with SEQ ID NO:342, and a sdCDR3 with SEQ ID NO:343. In some cases, the sdABD-HER2 has the amino acid sequence of SEQ ID NO:340, as provided in FIG. 2J.

In some embodiments, the sdABD-HER2 (e.g., sdABD-HER2 1150) has a sdCDR1 with SEQ ID NO:345, a sdCDR2 with SEQ ID NO:346, and a sdCDR3 with SEQ ID NO:347. In some cases, the sdABD-HER2 has the amino acid sequence of SEQ ID NO:344, as provided in FIG. 2J. In some embodiments, the sdABD-HER2 (e.g., sdABD-HER2 1156) has a sdCDR1 with SEQ ID NO:349, a sdCDR2 with SEQ ID NO:350, and a sdCDR3 with SEQ ID NO:351. In some cases, the sdABD-HER2 has the amino acid sequence of SEQ ID NO:348, as provided in FIG. 2J. In some embodiments, the sdABD-HER2 (e.g., sdABD-HER2 1158) has a sdCDR1 with SEQ ID NO:353, a sdCDR2 with SEQ ID NO:354, and a sdCDR3 with SEQ ID NO:355. In some cases, the sdABD-HER2 has the amino acid sequence of SEQ ID NO:352, as provided in FIG. 2J. In some embodiments, the sdABD-HER2 (e.g., sdABD-HER2 1159) has a sdCDR1 with SEQ ID NO:357, a sdCDR2 with SEQ ID NO:358, and a sdCDR3 with SEQ ID NO:359. In some cases, the sdABD-HER2 has the amino acid sequence of SEQ ID NO:356, as provided in FIG. 2J. In some embodiments, the sdABD-HER2 (e.g., sdABD-HER2 1160) has a sdCDR1 with SEQ ID NO:361, a sdCDR2 with SEQ ID NO:362, and a sdCDR3 with SEQ ID NO:363. In some cases, the sdABD-HER2 has the amino acid sequence of SEQ ID NO:360, as provided in FIG. 2J. In some embodiments, the sdABD-HER2 (e.g., sdABD-HER2 1161) has a sdCDR1 with SEQ ID NO:365, a sdCDR2 with SEQ ID NO:366, and a sdCDR3 with SEQ ID NO:367. In some cases, the sdABD-HER2 has the amino acid sequence of SEQ ID NO:364, as provided in FIG. 2K. In some embodiments, the sdABD-HER2 (e.g., sdABD-HER2 1162) has a sdCDR1 with SEQ ID NO:369, a sdCDR2 with SEQ ID NO:370, and a sdCDR3 with SEQ ID NO:371. In some cases, the sdABD-HER2 has the amino acid sequence of SEQ ID NO:368, as provided in FIG. 2K. In some embodiments, the sdABD-HER2 (e.g., sdABD-HER2 1163) has a sdCDR1 with SEQ ID NO:373, a sdCDR2 with SEQ ID NO:374, and a sdCDR3 with SEQ ID NO:375. In some cases, the sdABD-HER2 has the amino acid sequence of SEQ ID NO:372, as provided in FIG. 2K. In some embodiments, the sdABD-HER2 (e.g., humanized aHER2 sdAb h1139) has a sdCDR1 with SEQ ID NO:377, a sdCDR2 with SEQ ID NO:378, and a sdCDR3 with SEQ ID NO:379. In some cases, the sdABD-HER2 has the amino acid sequence of SEQ ID NO:376, as provided in FIG. 2K. In some embodiments, the sdABD-HER2 (e.g., humanized aHER2 sdAb h1156) has a sdCDR1 with SEQ ID NO:381, a sdCDR2 with SEQ ID NO:382, and a sdCDR3 with SEQ ID NO:383. In some cases, the sdABD-HER2 has the amino acid sequence of SEQ ID NO:380, as provided in FIG. 2K. In some embodiments, the sdABD-HER2 (e.g., humanized aHER2 sdAb h1159) has a sdCDR1 with SEQ ID NO:385, a sdCDR2 with SEQ ID NO:386, and a sdCDR3 with SEQ ID NO:387. In some cases, the sdABD-HER2 has the amino acid sequence of SEQ ID NO:384, as provided in FIG. 2K. In some embodiments, the sdABD-HER2 (e.g., humanized aHER2 sdAb h1162) has a sdCDR1 with SEQ ID NO:389, a sdCDR2 with SEQ ID NO:390, and a sdCDR3 with SEQ ID NO:391. In some cases, the sdABD-HER2 has the amino acid sequence of SEQ ID NO:388, as provided in FIG. 2L.
d) LyPD3 sdABDs

Additional embodiments of use in the invention are sdABDs to human LyPD3 as shown in FIGS. 2F-2G. In some embodiments, the sdABD-LyPD3 (e.g., sdABD-LyPD3 h787) has a sdCDR1 with SEQ ID NO:249, a sdCDR2 with SEQ ID NO:250, and a sdCDR3 with SEQ ID NO:251. In some cases, the sdABD-LyPD3 has the amino acid sequence of SEQ ID NO:248, as provided in FIG. 2F. In some embodiments, the sdABD-LyPD3 (e.g., sdABD-LyPD3 h790) has a sdCDR1 with SEQ ID NO:253, a sdCDR2 with SEQ ID NO:254, and a sdCDR3 with SEQ ID NO:255. In some cases, the sdABD-LyPD3 has the amino acid sequence of SEQ ID NO:252, as provided in FIG. 2F. In some embodiments, the sdABD-LyPD3 (e.g., sdABD-LyPD3 H804) has a sdCDR1 with SEQ ID NO:257, a sdCDR2 with SEQ ID NO:258, and a sdCDR3 with SEQ ID NO:259. In some cases, the sdABD-LyPD3 has the amino acid sequence of SEQ ID NO:256, as provided in FIG. 2F. In some embodiments, the sdABD-LyPD3 (e.g., sdABD-LyPD3 h773) has a sdCDR1 with SEQ ID NO:261, a sdCDR2 with SEQ ID NO:262, and a sdCDR3 with SEQ ID NO:263. In some cases, the sdABD-LyPD3 has the amino acid sequence of SEQ ID NO:260, as provided in FIG. 2F. In some embodiments, the sdABD-LyPD3 (e.g., sdABD-LyPD3 h840) has a sdCDR1 with SEQ ID NO:265, a sdCDR2 with SEQ ID NO:266, and a sdCDR3 with SEQ ID NO:267. In some cases, the sdABD-LyPD3 has the amino acid sequence of SEQ ID NO:264, as provided in FIG. 2F. In some embodiments, the sdABD-LyPD3 (e.g., sdABD-LyPD3 h885) has a sdCDR1 with SEQ ID NO:269, a sdCDR2 with SEQ ID NO:270, and a sdCDR3 with SEQ ID NO:271. In some cases, the sdABD-LyPD3 has the amino acid sequence of SEQ ID NO:268, as provided in FIG. 2G.
e) Trop2 sdABDs

Additional embodiments of use in the invention are sdABDs to human Trop2 as shown in FIGS. 2D-2E. In some embodiments, the sdABD-Trop2 (e.g., sdABD-Trop2 hVIB557) has a sdCDR1 with SEQ ID NO:78, a sdCDR2 with SEQ ID NO:79, and a sdCDR3 with SEQ ID NO:80. In some cases, the sdABD-Trop2 has the amino acid sequence of SEQ ID NO:77, as provided in FIG. 2D. In some embodiments, the sdABD-Trop2 (e.g., sdABD-Trop2 hVIB565) has a sdCDR1 with SEQ ID NO:82, a sdCDR2 with SEQ ID NO:83, and a sdCDR3 with SEQ ID NO:84. In some cases, the sdABD-Trop2 has the amino acid sequence of SEQ ID NO:81, as provided in FIG. 2D. In some embodiments, the sdABD-Trop2 (e.g., sdABD-Trop2 hVIB575) has a sdCDR1 with SEQ ID NO:86, a sdCDR2 with SEQ ID NO:87, and a sdCDR3 with SEQ ID NO:88. In some cases, the sdABD-Trop2 has the amino acid sequence of SEQ ID NO:85, as provided in FIG. 2D. In some embodiments, the sdABD-Trop2 (e.g., sdABD-Trop2 hVIB578) has a sdCDR1 with SEQ ID NO:90, a sdCDR2 with SEQ ID NO:91, and a sdCDR3 with SEQ ID NO:92. In some cases, the sdABD-Trop2 has the amino acid sequence of SEQ ID NO:89, as provided in FIG. 2D. In some embodiments, the sdABD-Trop2 (e.g., sdABD-Trop2 hVIB609) has a sdCDR1 with SEQ ID NO:94, a sdCDR2 with SEQ ID NO:95, and a sdCDR3 with SEQ ID NO:96. In some cases, the sdABD-Trop2 has the amino acid sequence of SEQ ID NO:93, as provided in FIG. 2D. In some embodiments, the sdABD-Trop2 (e.g., sdABD-Trop2 hVIB619) has a sdCDR1 with SEQ ID NO:98, a sdCDR2 with SEQ ID NO:99, and a sdCDR3 with SEQ ID NO:100. In some cases, the sdABD-Trop2 has the amino acid sequence of SEQ ID NO:97, as provided in FIG. 2E.
C. Human Serum Albumin (HSA) Domains

The MCE proteins of the invention (again, also referred to herein as “COBRA™” proteins or constructs) optionally include half-life extension domains such as HSA domains.

Human serum albumin (HSA) (molecular mass ˜67 kDa) is the most abundant protein in plasma, present at about 50 mg/ml (600 uM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise an HSA domain that is all or part of the full length HSA molecule, the sequence of which is shown in FIG. 2L. In addition, truncated and/or variant versions of HSA can be used, as long as the pH sensitive binding to FcRn is retained, which can be assessed by binding assays such as Octet. Suitable HSA truncations and HSA variants are known in the art. See, for example, U.S. Pat. No. 10,711,050, the contents of which are incorporated herein by reference in its entirety and specifically for the HSA variants and binding constants outlined therein including those in the sequence listing and figures. See also Sand et al., JBC 289(5):34583 (2014), incorporated herein by reference in its entirety and specifically for the 4 HSA variants that showed increased binding to FcRn (N109A, N111A, L112A and P113A).

In some embodiments, the HSA domain has at least 90% (e.g., 90%, 91%, 92%, 93%, 94%, 95%, 96%, 97%, 98%, 99% or more) sequence identity to the amino acid sequence of SEQ ID NO:117. In some embodiments, the HSA domain has the amino acid sequence of SEQ ID NO:117. In some embodiments, the HSA domain is a variant HSA domain comprising the amino acid sequence of SEQ ID NO:117 and one or more (e.g., 1, 2, 3, 4, 5, 6, 7, 8, 9, 10 or more) amino acid modifications (e.g., substitution, addition and/or deletion). In some embodiments, the HSA domain is a variant HSA domain comprising the amino acid sequence of SEQ ID NO:117 and one or more amino acid modifications at positions V418, T420, V424, N429, M446, A449, T467, E505, V547 and/or A552. In some embodiments, the HSA domain is a variant HSA domain comprising the amino acid sequence of SEQ ID NO:117 and one or more amino acid substitutions selected from the group consisting of V418M, T420A, V4241, N429D, M446V, A449V, T467M, E505R, E505K, E505G, V547A and A552T. In some embodiments, the HSA domain has at least 90% (e.g., 90%, 91%, 92%, 93%, 94%, 95%, 96%, 97%, 98%, 99% or more) sequence identity to the amino acid sequence of SEQ ID NO:2 of U.S. Pat. No. 10,711,050. In some embodiments; the HSA domain has the amino acid sequence of SEQ ID NO:2 of U.S. Pat. No. 10,711,050. In some embodiments, the HSA domain is a variant HSA domain comprising the amino acid sequence of SEQ ID NO:2 of U.S. Pat. No. 10,711,050 and one or more (e.g., 1, 2, 3, 4, 5, 6, 7, 8, 9, 10 or more) amino acid modifications (e.g., substitution, addition and/or deletion). In some embodiments, the HSA domain is a variant HSA domain comprising the amino acid sequence of SEQ ID NO:2 of U.S. Pat. No. 10,711,050 and one or more amino acid modifications at positions V418, T420, V424, N429, M446, A449, T467, E505, V547 and/or A552. In some embodiments, the HSA domain is a variant HSA domain comprising the amino acid sequence of SEQ ID NO:2 of U.S. Pat. No. 10,711,050 and one or more amino acid substitutions selected from the group consisting of V418M, T420A, V424I, N429D, M446V, A449V, T467M, E505R, E505K, E505G, V547A and A552T.

The HSA domain of the MCE constructs of the invention provides for altered pharmacodynamics and pharmacokinetics of the construct itself. As above, the HSA domain extends the elimination half-time. The HSA domain also alters pharmacodynamic properties including alteration of tissue distribution, penetration, and diffusion of the antigen-binding protein. In some embodiments, the HSA domain provides for improved tissue (including tumor) targeting, tissue penetration, tissue distribution, diffusion within the tissue, and enhanced efficacy as compared with a protein without a HSA domain. In one embodiment, therapeutic methods effectively and efficiently utilize a reduced amount of the construct of the invention, resulting in reduced side effects, such as reduced non-tumor cell cytotoxicity or increased dosing intervals (e.g., less frequent dosing). Methods of evaluating the metabolism and pharmacokinetics of any of the prodrug constructs described herein comprising a human serum albumin domain including variants and derivatives thereof can be tested in albumin-deficient mouse models as described in Roopenian et al., mAbs, 2015, 7(2): 344-351, the disclosure of which is incorporated herein in its entirety.
D. Protease Cleavage Sites

The protein compositions of the invention, and particularly the prodrug constructs, include one or more protease cleavage sites, generally resident in cleavable linkers, as outlined herein.

As described herein, the prodrug constructs of the invention include at least one protease cleavage site comprising an amino acid sequence that is cleaved by at least one protease. In some cases, the MCE proteins described herein comprise 1, 2, 3, 4, 5, 6, 7, 8, 9, 10, 11, 12, 13, 14, 15, 16, 17, 18, 19, 20, or more protease cleavage sites that are cleaved by at least one protease. As is more fully discussed herein, when more than one protease cleavage site is used in a prodrug construction, they can be the same (e.g., multiple sites that are cleaved by a single protease) or different (two or more cleavage sites are cleaved by at least two different proteases). As will be appreciated by those in the art, constructs containing three or more protease cleavage sites can utilize one, two, three, etc.; e.g., some constructs can utilize three sites for two different proteases, etc.

The amino acid sequence of the protease cleavage site will depend on the protease that is targeted. As is known in the art, there are a number of human proteases that are found in the body and can be associated with disease states.

Proteases are known to be secreted by some diseased cells and tissues, for example tumor or cancer cells, creating a microenvironment that is rich in proteases or a protease-rich microenvironment. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, Cathepsins (e.g., cathepsin B, cathepsin C, cathepsin D, cathepsin E, cathepsin K, cathepsin L, and cathepsin S), kallikreins, hK1, hK10, hK15, KLK7, GranzymeB, plasmin, collagenase, type IV collagenase, stromelysin, factor XA, chymotrypsin-like protease, trypsin-like protease, elastase-like protease, subtilisin-like protease, actinidain, bromelain, calpain, caspases (e.g., caspase-3), Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMPI, MMP2, MMP3, MMP8, MMP9, MMP13, MMP11, MMP14, meprin, urokinase plasminogen activator (uPA), enterokinase, prostate-specific antigen (PSA, hK3), interleukin-10 converting enzyme, thrombin, FAP alpha (FAP-a), dipeptidyl peptidase, and dipeptidyl peptidase IV (DPPIV/CD26). Some suitable proteases and protease cleavage sequences are shown in FIGS. 3A-3D.
E. Linkers

As is discussed herein, the different domains of the invention are generally linked together using amino acid linkers, which can confer functionality as well, including flexibility or inflexibility (e.g. steric constraint) as well as the ability to be cleaved using an in situ protease. These linkers can be classified in a number of ways.

The invention provides “domain linkers”, which are used to join two or more domains (e.g. a VH and a VL, a target tumor antigen binding domain (TTABD, sometimes also referred to herein as “αTTA” (for “anti-TTA”) to a VH or VL, an HSA domain to another component, etc. Domain linkers can be non-cleavable (NCL), cleavable (“CL”), constrained and cleavable (CCL) and constrained and non-cleavable (CNCL), for example.

1. Non-Cleavable Linkers

In some embodiments, the domain linker is non-cleavable. Generally, these can be one of two types: non-cleavable and flexible, allowing for the components “upstream” and “downstream” of the linker in the constructs to intramolecularly self-assemble in certain ways; or non-cleavable and constrained, where the two components separated by the linker are not able to intramolecularly self-assemble. It should be noted, however, that in the latter case, while the two component domains that are separated by the non-cleavable constrained linker do not intramolecularly self-assemble, other intramolecular components will self-assemble to form the pseudo Fv domains.

### (i) Non-cleavable but Flexible Linkers

In this embodiment, the linker is used to join domains to preserve the functionality of the domains, generally through longer, flexible domains that are not cleaved by in situ proteases in a patient. Examples of internal, non-cleavable linkers suitable for linking the domains in the polypeptides of the invention include but are not limited to (GS)n, (GGS)n, (GGGS)n [SEQ ID NO:244], (GGSG)n [SEQ ID NO:245], (GGSGG)n [SEQ ID NO:246], or (GGGGS)n [SEQ ID NO:247], wherein n is 1, 2, 3, 4, 5, 6, 7, 8, 9, or 10. In some embodiments the length of the linker can be about 15 amino acids.

### (ii) Non-Cleavable and Constrained Linkers

In some cases, the linkers do not contain a cleavage site and are also too short to allow the protein domains separated by the linker to intramolecularly self-assemble, and are “constrained non-cleavable linkers” or “CNCLs”. For example, in Pro817, an active VH and an active VL are separated by 8 amino acids (an “8-mer”) that does not allow the VH and VL to self-assemble into an active antigen binding domain. In some embodiments, the linker is still flexible; for example, (GGGS)n where n=2. In other embodiments, although generally less preferred, more rigid linkers can be used, such as those that include proline or bulky amino acids.

2. Cleavable Linkers

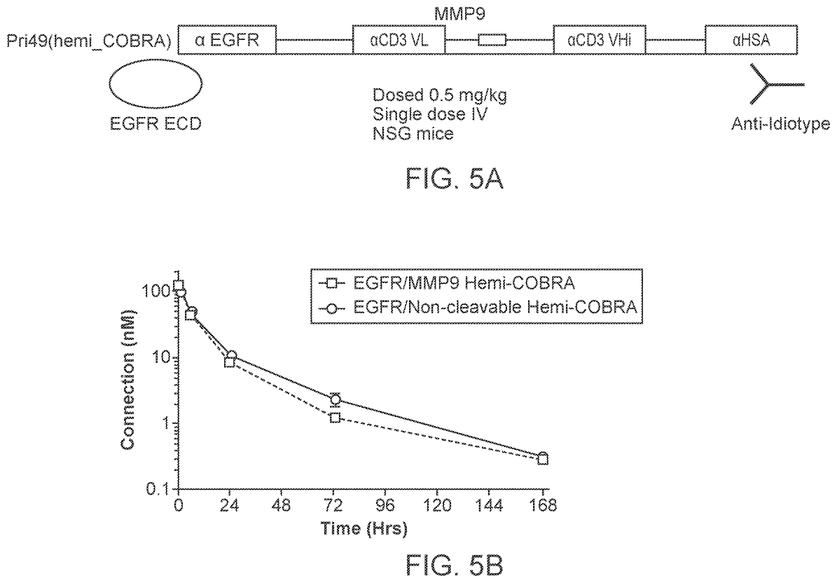

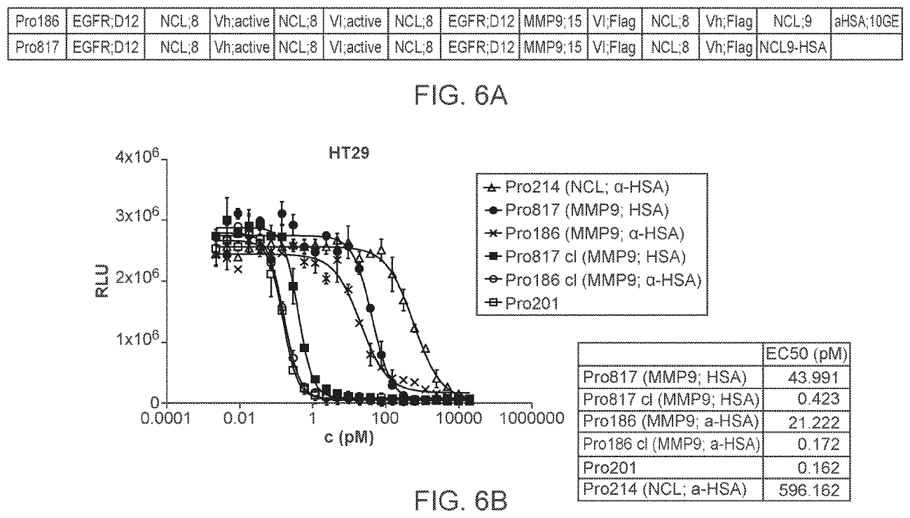

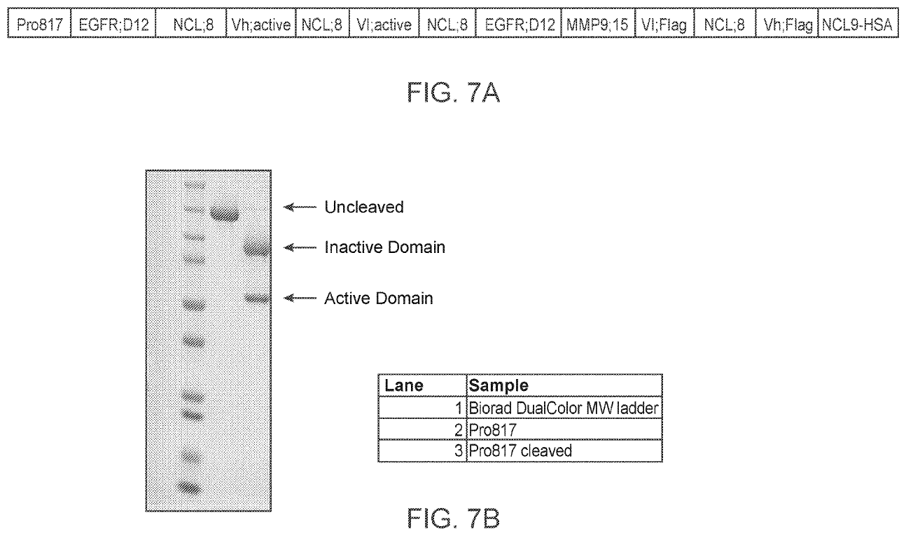

All of the prodrug constructs herein include at least one cleavable linker. Thus, in one embodiment, the domain linker is cleavable (CL), sometimes referred to herein as a “protease cleavage domain” (“PCD”). In this embodiment, the CL contains a protease cleavage site, as outlined herein and as depicted in FIG. 5 and FIG. 6. In some cases, the CL contains just the protease cleavage site. Optionally, depending on the length of the cleavage recognition site, there can be an extra few linking amino acids at either or both of the N- or C-terminal end of the CL; for example, there may be from 1, 2, 3, 4 or 5 amino acids on either or both of the N- and C-termini of the cleavage site. Thus, cleavable linkers can also be constrained (e.g,. 8-mers) or flexible.

Of particular interest in the present invention are MMP9 cleavable linkers and meprin cleavable linkers, particularly MMP9 constrained cleavable linkers and meprin constrained cleavable linkers.
F. Domains of the Invention

The present invention provides a number of different formats for the prodrug polypeptides of the invention. The present invention provides constrained Fv domains and constrained pseudo Fv domains. Additionally, the present invention provides multivalent conditionally effective (“MCE”) proteins which contain two Fv domains but are non-isomerizing constructs. As outlined herein, these can be non-isomerizing cleavable formats or non-isomerizing non-cleavable formats, although every construct contains at least one protease cleavage domain.

Importantly, while both of these domains (Fv domains and pseudo Fv domains) are referred to herein as “constrained”, meaning that as discussed above and shown in FIG. 37, FIG. 38 and FIG. 39 of US Pub. No. 2019/0076524, only one of these needs to be constrained, although generally, when both linkers are constrained, the protein has better expression.

Those of skill in the art will appreciate that for pro-drug format 2, there are four possibilities for the N- to C-terminal order of the constrained and pseudo Fv domains of the invention (not showing the linkers): aVH-aVL and iVL-iVH, aVH-aVL and iVH-iVL, aVL-aVH and iVL-iVH, aVL-aVH and iVH-iVL, wherein “aVH” refers to an active VH domain, “aVL” refers to an active VL domain, “iVH” refers to an inactive VH domain, “iVL” refers to an inactive VL domain. All four have been tested and all four have activity, although the first order, aVH-aVL and iVL-iVH, shows better expression than the other three. Thus while the description herein is generally shown in this aVH-aVL and iVL-iVH format, all disclosure herein includes the other orders for these domains as well.

Note that generally, the N to C-terminal order for the full length constructs of the invention is based on the aVH-aVL and iVL-iVH orientation.

Additionally, it is known in the art that there can be immunogenicity in humans originating from the C-terminal sequences of certain ABDs. Accordingly, in general, particularly when the C-terminus of the constructs terminates in an sdABD (for example, the sdABD-HSA domains of many of the constructs, a histidine tag (either His6 or His10) can be used. Many or most of the sequences herein were generated using His6 C-terminal tags for purification reasons, but these sequences can also be used to reduce immunogenicity in humans, as is shown by Holland et al., J Clin Immunol, 2013, 33: 1192-1203 and WO2013/024059.
G. Constrained Fv domains

The present invention provides constrained Fv domains, that comprise an active VH and an active VL domain that are covalently attached using a constrained linker. The constrained linker prevents intramolecular association between the aVH and aVL in the absence of cleavage. Thus, a constrained Fv domain general comprises a set of six CDRs contained within variable domains, wherein the vhCDR1, vhCDR2 and vhCDR3 of the VH bind human CD3 and the v1CDR1, vCDR2 and v1CDR3 of the VL bind human CD3, but in the prodrug format (e.g., uncleaved), the VH and VL are unable to sterically associate to form an active binding domain, preferring instead to pair intramolecularly with the pseudo Fv.

The constrained Fv domains can comprise active VH and active VL (aVH and aVL) or inactive VH and VL (iVH and iVL), in which case it is a constrained pseudo Fv domain) or combinations thereof as described herein.

As will be appreciated by those in the art, the order of the VH and VL in a constrained Fv domain can be either (N- to C-terminal) VH-linker-VL or VL-linker-VH.

As outlined herein, the constrained Fv domains can comprise a VH and a VL linked using a non-cleavable linker. In this embodiment, the constrained Fv domain has the structure (N- to C-terminus) vhFR1-vhCDR1-vhFR2-vhCDR2-vhFR3-vhCDR3-vhFR4-CNCL-v1FR1-v1CDR1-v1FR2-v1CDR2-v1FR3-v1CDR3-v1FR4. In general, the constrained Fv domain contains active VH and VL domains (e.g., able to bind CD3 when associated) and thus has the structure (N- to C-terminus) vhFR1-avhCDR1-vhFR2-avhCDR2-vhFR3-avhCDR3-vhFR4-CNCL-v1FR1-av1CDR1-v1FR2-av1CDR2-v1FR3-av1CDR3-v1FR4.

Of particular use in the present invention are constrained non-cleavable Fv domains having an aVH having SEQ ID NO:134, an aVL having SEQ ID NO:118, and a domain linker having SEQ ID NO:151.
H. Constrained Pseudo Fv Domains

The present invention provides constrained pseudo Fv domains, comprising inactive or pseudo iVH and iVL domains that are covalently attached using a constrained linker (which, as outlined herein, can be cleavable or non-cleavable). The constrained linker prevents intramolecular association between the iVH and iVL in the absence of cleavage. Thus, a constrained pseudo Fv domain general comprises an iVH and an iVL with framework regions that allow association (when in a non-constrained format) of the iVH and iVL, although the resulting pseudo Fv domain does not bind to a human protein. iVH domains can assemble with aVL domains, and iVL domains can assemble with aVH domains, although the resulting structures do not bind to CD3.

The constrained pseudo Fv domains comprise inactive VH and VL (iVH and iVL) domains. See, for example, FIGS. 2M and 2N. Examples of inactive VH domains include SEQ ID NOS:138, 142 and 146. Examples of inactive VL domains include SEQ ID NOS:122, 126 and 130.

As will be appreciated by those in the art, the order of the VH and VL in a constrained pseudo Fv domain can be either (N- to C-terminal) VH-linker-VL or VL-linker-VH.

As outlined herein, the constrained pseudo Fv domains can comprise an iVH and an iVL linked using a non-cleavable linker.

In general, the constrained Fv domain contains inert VH and VL domains (e.g. able to bind CD3 when associated) and thus has the structure (N- to C-terminus) vhFR1-iv1CDR1-vhFR2-iv1CDR2-vhFR3-iv1CDR3-vhFR4-CNCL-v1FR1-ivhCDR1-v1FR2-ivhCDR2-v1FR3-ivhCDR3-v1FR4.

Of particular use in the present invention are constrained non-cleavable pseudo Fv domains having an iVH having SEQ ID NO:138, SEQ ID NO:142 or SEQ ID NO:146, an iVL having SEQ ID NO:122, SEQ ID NO:126, or SEQ ID NO:130, and a domain linker having SEQ ID NO:151.

In some embodiments are constrained non-cleavable pseudo Fv domains having an iVH having SEQ ID NO:138 and an iVL having SEQ ID NO:122. In some embodiments are constrained non-cleavable pseudo Fv domains having an iVH having SEQ ID NO:142 and an iVL having SEQ ID NO:126. In some embodiments are constrained non-cleavable pseudo Fv domains having an iVH having SEQ ID NO:146 and an iVL having SEQ ID NO:130.
IV. Formats

As discussed herein, the invention provides non-cleavable formats. In this embodiment, it is understood that the “non-cleavable” applies only to the linkage of the constrained Fv domain, as there is the activating cleavage site in the prodrug construct. In this embodiment, the constrained Fv domain comprise VH and VL domains that are linked using constrained non-cleavable linkers and the constrained pseudo Fv domain uses constrained non-cleavable linkers (CNCL).

As will be appreciated by those in the art, the order of the VH and VL in either a constrained Fv domain or a constrained pseudo Fv domain can be either (N- to C-terminal) VH-linker-VL or VL-linker-VH.

The invention provides prodrug proteins, comprising, from N- to C-terminal, (sdABD-TTA1)-domain linker-constrained Fv domain-domain linker-(sdABD-TTA2)-cleavable linker-constrained pseudo Fv domain-domain linker-HSA domain.

As will be appreciated by those in the art, the order of the VH and VL in either a constrained Fv domain or a constrained pseudo Fv domain can be either (N- to C-terminal) VH-linker-VL or VL-linker-VH.

Thus, in one embodiment, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain.

Thus, in one embodiment, the prodrug protein comprises, from N- to C-terminal: (sdABD-TTA1)-domain linker-aVH-CNCL-aVL-domain linker-(sdABD-TTA2)-CL-iVH-CNCL-iVL-domain linker-HSA-domain.

Thus, in one embodiment, the prodrug protein comprises, from N- to C-terminal: (sdABD-TTA1)-domain linker-aVL-CNCL-aVH-domain linker-(sdABD-TTA2)-CL-iVL-CNCL-iVH-domain linker-HSA domain.

Thus, in one embodiment, the prodrug protein comprises, from N- to C-terminal: (sdABD-TTA1)-domain linker-aVL-CNCL-aVH-domain linker-(sdABD-TTA2)-CL-iVH-CNCL-iVL-domain linker-HSA-domain.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain. In this embodiment, the aVH, aVL, iVH, iVL have the sequences shown in FIGS. 2M and 2N. In this embodiment, the two targeting domains bind to the same TTA, which can be B7H3, CA9, EGFR, EpCAM, FOLR1, HER2, LyPD3, or Trop2, the sequences for which are depicted in FIGS. 2A-2L and the formal sequence listing.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain. In this embodiment, the aVH, aVL, iVH, iVL have the sequences shown in FIG. 2. In this embodiment, the two targeting domains bind to different TTAs. In some embodiments, the sdABD-TTA1 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA2 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA1 is an sdABD-B7H3 and the sdABD-TTA2 is selected from the group consisting of an sdABD-CA9, sdABD-EGFR, sdABD-EpCAM, sdABD-FOLR1, sdABD-HER2, sdABD-LyPD3, and sdABD-Trop2, or the reverse. In some embodiments, the sdABD-TTA1 is an sdABD-CA9 and the sdABD-TTA2 is selected from the group consisting of an sdABD-B7H3, sdABD-EGFR, sdABD-EpCAM, sdABD-FOLR1, sdABD-HER2, sdABD-LyPD3, and sdABD-Trop2, or the reverse. In some embodiments, the sdABD-TTA1 is an sdABD-EGFR and the sdABD-TTA2 is selected from the group consisting of an sdABD-B7H3, sdABD-CA9, sdABD-EpCAM, s sdABD-FOLR1, dABD-HER2, sdABD-LyPD3, and sdABD-Trop2, or the reverse. In some embodiments, the sdABD-TTA1 is an sdABD-EpCAM and the sdABD-TTA2 is selected from the group consisting of an sdABD-B7H3, sdABD-CA9, sdABD-EGFR, sdABD-FOLR1, sdABD-HER2, sdABD-LyPD3, and sdABD-Trop2, or the reverse. In some embodiments, the sdABD-TTA1 is an sdABD-FOLR1 and the sdABD-TTA2 is selected from the group consisting of an sdABD-B7H3, sdABD-CA9, sdABD-EGFR, sdABD-EpCAM, sdABD-HER2, sdABD-LyPD3, and sdABD-Trop2, or the reverse. In some embodiments, the sdABD-TTAI is an sdABD-HER2 and the sdABD-TTA2 is selected from the group consisting of an sdABD-B7H3, sdABD-CA9, sdABD-EGFR, sdABD-EpCAM, sdABD-FOLR1, sdABD-LyPD3, and sdABD-Trop2, or the reverse. In some embodiments, the sdABD-TTA1 is an sdABD-LyPD3 and the sdABD-TTA2 is selected from the group consisting of an sdABD-B7H3, sdABD-CA9, sdABD-EGFR, sdABD-EpCAM, sdABD-FOLR1, sdABD-HER2, and sdABD-Trop2, or the reverse. In some embodiments, the sdABD-TTA1 is an sdABD-Trop2 and the sdABD-TTA2 is selected from the group consisting of an sdABD-B7H3, sdABD-CA9, sdABD-EGFR, sdABD-EpCAM, sdABD-FOLR1, sdABD-HER2, and sdABD-LyPD3, or the reverse.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain, and the sdABD-TTA1 and sdABD-TTA2 bind the same target antigen. In some embodiments, the sdABD-TTA1 and the sdABD-TTA2 bind the same target antigen but at different locations. In some embodiments, the sdABD-TTA1 and the sdABD-TTA2 bind the same target antigen but at the same location. In some embodiments, the sdABD-TTA1 and the sdABD-TTA2 have the same amino acid sequence.

Any sequence of the sdABDs described herein can be the sequence of the sdABD-TTA1, the sdABD-TTA2, or both. In some embodiments, the sdCDR1, sdCDR2 and sdCDR3 of sdABD-TTA1 are the same as the the sdCDR1, sdCDR2 and sdCDR3 of sdABD-TTA2, respectively.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain. In this embodiment, the aVH, aVL, iVH, iVL have the sequences shown in FIG. 2. In some embodiments, the two targeting domains bind to the TTA pairs: B7H3 and CA9, B7H3 and EGFR, B7H3 and EpCAM, B7H3 and FOLR1, B7H3 and HER2, B7H3 and LyPD3, B7H3 and Trop2, CA9 and EGFR, CA9 and EpCAM. CA9 and FOLR1, CA9 and HER2, CA9 and LyPD3, CA9 and Trop2, EGFR and EpCAM, EGFR and FOLR1, EGFR and HER2, EGFR and LyPD3, EGFR and Trop2, EpCAM and FOLR1, EpCAM and HER2, EpCAM and LyPD3, EpCAM and Trop2, FOLR1 and HER2, FOLR1 and LyPD3, FOLR1 and Trop2, HER2 and LyPD3, HER2 and Trop2, and LyPD3 and Trop2, and the sdABD-TTAs have the sequences in FIG. 2.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain. In this embodiment, the aVH, aVL, iVH, iVL have the sequences shown in FIG. 2. In this embodiment, the two targeting domains bind to the same TTA, which can be B7H3, CA9, EGFR, EpCAM, FOLR1, HER2, LyPD3, or Trop2, the sequences for which are depicted in FIG. 2, and the CCL and CL is selected from a linker that is cleaved by MMP9 or meprin, and the HSA domain has an amino acid sequence of SEQ ID NO:117.

In these embodiments, a preferred domain linker has the amino acid sequence of SEQ ID NO:151 (which also serves as a preferred constrained non cleavable linker).
I. Single Targeting Format 2 Constructs: “Mono-specific COBRAs”

In some embodiments, both of the αTTA domains bind to the same tumor target antigen (TTA). Accordingly, in some embodiments, the prodrug protein comprises, from N- to C-terminal: (sdABD-TTA1)-domain linker-aVH-CNCL-aVL-domain linker-sdABD-TTA2)-CL-iVL-CNCL-iVH-domain linker-HSA domain. In this embodiment, the aVH, aVL, iVH, iVL have the sequences shown in FIGS. 2M-2N. In this embodiment, the two targeting domains bind to the same TTA, which can be B7H3, CA9, EGFR, EpCAM, FOLR1, HER2, LyPD3, or Trop2, the sequences for the sdABD-TTAs are depicted in FIGS. 2A-2L.

In some embodiments, the sdABD-TTA1 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA2 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA1 and sdABD-TTA2 bind the same tumor target antigen. In some embodiments, the sdABD-TTA1 and the sdABD-TTA2 bind the same tumor target antigen but at different locations. In some embodiments, the sdABD-TTA1 and the sdABD-TTA2 bind the same tumor target antigen, but at the same location of the TTA. In some embodiments, the sdABD-TTA1 and the sdABD-TTA2 have the same amino acid sequence. Any sequence of the sdABDs described herein can be the sequence of the sdABD-TTA1, the sdABD-TTA2, or both. In some embodiments, the sdCDRs of sdABD-TTA1 are the same as the sdCDRs of sdABD-TTA2.
J. Dual Targeting Format 2 Constructs: “HeteroCOBRAs”

In some embodiments, each of the αTTA domains bind to a different tumor target. Accordingly, in some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-HSA domain. In some embodiments, the aVH, aVL, iVH iVL have the sequences shown in FIGS. 2M-2N. In some embodiments, the two targeting domains bind to different TTAs.

In some embodiments, the first TTA (TTA1) and the second TTA (TTA2) are different. In some embodiments, the first TTA and the second TTA are selected from the group consisting of B7H3, CA9, EGFR, EpCAM, FOLR1, HER2, LyPD3, Trop2, and any combination thereof.

In some embodiments of the prodrug protein and/or the cleaved protein, the first TTA is B7H3 and the second TTA (TTA2) is selected from the group consisting of CA9, EGFR, EpCAM, FOLR1, HER2, LyPD3, and Trop2. In some embodiments of the prodrug protein and/or the cleaved protein, the first TTA is CA9 and the second TTA is selected from the group consisting of B7H3, EGFR, EpCAM, FOLR1, HER2, LyPD3 and Trop2. In some embodiments of the prodrug protein and/or the cleaved protein, the first TTA is EGFR and the second TTA is selected from the group consisting of B7H3, CA9, EpCAM, FOLR1, HER2, LyPD3 and Trop2. In some embodiments of the prodrug protein and/or the cleaved protein, the first TTA is EpCAM and the second TTA is selected from the group consisting of B7H3, CA9, EGFR, FOLR1, HER2, LyPD3 and Trop2. In some embodiments of the prodrug protein and/or the cleaved protein, the first TTA is FOLR1 and the second TTA is selected from the group consisting of B7H3, CA9, EGFR, EpCAM, HER2, LyPD3 and Trop2. In some embodiments of the prodrug protein and/or the cleaved protein, the first TTA is HER2 and the second TTA is selected from the group consisting of B7H3, CA9, EGFR, EpCAM, FOLR1, LyPD3 and Trop2. In some embodiments of the prodrug protein and/or the cleaved protein, the first TTA is LyPD3 and the second TTA is selected from the group consisting of B7H3, CA9, EGFR, EpCAM, FOLR1, HER2, and Trop2. In some embodiments of the prodrug protein and/or the cleaved protein, the first TTA is Trop2 and the second TTA is selected from the group consisting of B7H3, CA9, EGFR, EpCAM, FOLR1, HER2, and LyPD3.

In some embodiments of the prodrug protein and/or the cleaved protein, the first TTA is selected from the group consisting of CA9, EGFR, EpCAM, FOLR1, HER2, LyPD3 and Trop2 and the second TTA is B7H3. In some embodiments of the prodrug protein and/or the cleaved protein, the first TTA is selected from the group consisting of B7H3, EGFR, EpCAM, FOLR1, HER2, LyPD3 and Trop2 and the second TTA is CA9. In some embodiments of the prodrug protein and/or the cleaved protein, the first TTA is selected from the group consisting of B7H3, CA9, EpCAM, FOLR1, HER2, LyPD3 and Trop2 and the second TTA (TTA2) is EGFR. In some embodiments of the prodrug protein and/or the cleaved protein, the first TTA is selected from the group consisting of B7H3, CA9, EGFR, FOLR1, HER2, LyPD3 and Trop2 and the second TTA is EpCAM. In some embodiments of the prodrug protein and/or the cleaved protein, the first TTA is selected from the group consisting of B7H3, CA9, EGFR, EpCAM, HER2, LyPD3 and Trop2 and the second TTA is FOLR1. In some embodiments of the prodrug protein and/or the cleaved protein, the first TTA is selected from the group consisting of B7H3, CA9, EGFR, EpCAM, FOLR1, LyPD3 and Trop2 and the second TTA is HER2. In some embodiments of the prodrug protein and/or the cleaved protein, the first TTA is selected from the group consisting of B7H3, CA9, EGFR, EpCAM, FOLR1, HER2, and Trop2 and the second TTA is LyPD3. In some embodiments of the prodrug protein and/or the cleaved protein, the first TTA is selected from the group consisting of B7H3, CA9, EGFR, EpCAM, FOLR1, HER2, and LyPD3 and the second TTA is Trop2.

In some embodiments, the sdABD-TTA1 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA2 is selected from the group consisting of an sdABD-B7H3, sdABD-CA9, sdABD-EGFR, sdABD-EpCAM, sdABD-FOLR1, sdABD-HER2, sdABD-LyPD3, and sdABD-Trop2. In some embodiments, the sdABD-TTA1 and sdABD-TTA2 bind different target antigens. In some embodiments, the sdABD-B7H3 comprises an amino sequence selected from the group consisting of SEQ ID NOS:33, 37, 41, 45, 49, 53 and 57. In some embodiments, the sdABD-CA9 comprises an amino sequence selected from the group consisting of SEQ ID NOS: 101, 105, 109, and 113. In some embodiments, the sdABD-EGFR comprises an amino sequence selected from the group consisting of SEQ ID NOS:1, 5, 9, 13 and 17. In some embodiments, the sdABD-EpCAM comprises an amino sequence selected from the group consisting of SEQ ID NOS:61, 65, 69, 73, and 392. In some embodiments, the sdABD-FOLR1 comprises an amino sequence selected from the group consisting of SEQ ID NOS:21, 25 and 29. In some embodiments, the sdABD-HER2 comprises an amino sequence selected from the group consisting of SEQ ID NOS:272, 276, 280, 284, 288, 292, 296, 300, 304, 308, 312, 316, 320, 324, 328, 332, 336, 340, 344, 348, 352, 356, 360, 364, 368, 372, 376, 380, 384, and 388. In some embodiments, the sdABD-LyPD3 comprises an amino sequence selected from the group consisting of SEQ ID NOS:248, 252, 256, 260, 264, and 268. In some embodiments, the sdABD-Trop2 comprises an amino sequence selected from the group consisting of SEQ ID NOS:77, 81, 85, 89, 93, and 97.

In some embodiments, the sdABD-TTA1 is an sdABD-B7H3 and the sdABD-TTA2 is selected from the group consisting of an sdABD-CA9, sdABD-EGFR, sdABD-EpCAM, sdABD-FOLR1, sdABD-HER2, sdABD-LyPD3, and sdABD-Trop2. In some embodiments, the sdABD-TTA1 is an sdABD-CA9 and the sdABD-TTA2 is selected from the group consisting of an sdABD-B7H3, sdABD-EGFR, sdABD-EpCAM, sdABD-FOLR1, sdABD-HER2, sdABD-LyPD3, and sdABD-Trop2. In some embodiments, the sdABD-TTA1 is an sdABD-EGFR and the sdABD-TTA2 is selected from the group consisting of an sdABD-B7H3, sdABD-CA9, sdABD-EpCAM, sdABD-FOLR1, sdABD-HER2, sdABD-LyPD3, and sdABD-Trop2. In some embodiments, the sdABD-TTA1 is an sdABD-EpCAM and the sdABD-TTA2 is selected from the group consisting of an sdABD-B7H3, sdABD-CA9, sdABD-EGFR, sdABD-FOLR1, sdABD-HER2, sdABD-LyPD3, and sdABD-Trop2. In some embodiments, the sdABD-TTA1 is an sdABD- FOLR1 and the sdABD-TTA2 is selected from the group consisting of an sdABD-B7H3, sdABD-CA9, sdABD-EGFR, sdABD-EpCAM, sdABD-HER2, sdABD-LyPD3, and sdABD-Trop2. In some embodiments, the sdABD-TTA1 is an sdABD-HER2 and the sdABD-TTA2 is selected from the group consisting of an sdABD-B7H3, sdABD-CA9, sdABD-EGFR, sdABD-EpCAM, sdABD-FOLR1, sdABD-LyPD3, and sdABD-Trop2. In some embodiments, the sdABD-TTA1 is an sdABD-LyPD3 and the sdABD-TTA2 is selected from the group consisting of an sdABD-B7H3, sdABD-CA9, sdABD-EGFR, sdABD-EpCAM, sdABD-FOLR1, sdABD-HER2, and sdABD-Trop2. In some embodiments, the sdABD-TTA1 is an sdABD-Trop2 and the sdABD-TTA2 is selected from the group consisting of an sdABD-B7H3, sdABD-CA9, sdABD-EGFR, sdABD-EpCAM, sdABD-FOLR1, sdABD-HER2, and sdABD-LyPD3. Any sequence of an sdABD-TTA described herein such as those of an sdABD-B7H3, an sdABD-CA9, an sdABD-EGFR, an sdABD-EpCAM, an sdABD-FOLR1, an sdABD-HER2, an sdABD-LyPD3 and an sdABD-Trop2 can be used in a dual targeting format 2 construct or hetero-COBRA.

In many embodiments, the sdABD-TTA1 is selected from the group consisting of an sdABD-CA9, sdABD-EGFR, sdABD-EpCAM, sdABD-FOLR1, sdABD-HER2, sdABD-LyPD3, and sdABD-Trop2, and the sdABD-TTA2 is an sdABD-B7H3. In many embodiments, the sdABD-TTA1 is selected from the group consisting of an sdABD-B7H3, sdABD-EGFR, sdABD-EpCAM, sdABD-FOLR1, sdABD-HER2, sdABD-LyPD3, and sdABD-Trop2, and the sdABD-TTA2 is an sdABD-CA9. In many embodiments, the sdABD-TTA1 is selected from the group consisting of an sdABD-B7H3, sdABD-CA9, sdABD-EpCAM, sdABD-FOLR1, sdABD-HER2, sdABD-LyPD3, and sdABD-Trop2, and the sdABD-TTA2 is an sdABD-EGFR. In many embodiments, the sdABD-TTA1 is selected from the group consisting of an sdABD-B7H3, sdABD-CA9, sdABD-EGFR, sdABD-FOLR1, sdABD-HER2, sdABD-LyPD3, and sdABD-Trop2, and the sdABD-TTA2 is an sdABD-EpCAM. In many embodiments, the sdABD-TTA1 is selected from the group consisting of an sdABD-B7H3, sdABD-CA9, sdABD-EGFR, sdABD-EpCAM, sdABD-HER2, sdABD-LyPD3, and sdABD-Trop2, and the sdABD-TTA2 is an sdABD-FOLR1. In many embodiments, the sdABD-TTA1 is selected from the group consisting of an sdABD-B7H3, sdABD-CA9, sdABD-EGFR, sdABD-EpCAM, sdABD-FOLR1, sdABD-LyPD3, and sdABD-Trop2, and the sdABD-TTA2 is an sdABD-HER2. In many embodiments, the sdABD-TTA1 is selected from the group consisting of an sdABD-B7H3, sdABD-CA9, sdABD-EGFR, sdABD-EpCAM, sdABD-FOLR1, sdABD-HER2, and sdABD-Trop2, and the sdABD-TTA2 is an sdABD-LyPD3. In many embodiments, the sdABD-TTA1 is selected from the group consisting of an sdABD-B7H3, sdABD-CA9, sdABD-EGFR, sdABD-EpCAM, sdABD-FOLR1, sdABD-HER2, and sdABD-LyPD3, and the sdABD-TTA2 is an sdABD-Trop2. Any sequence of an sdABD-TTA described herein such as those of an sdABD-B7H3, an sdABD-CA9, an sdABD-EGFR, an sdABD-EpCAM, an sdABD-HER2, an an sdABD-LyPD3 and an sdABD-Trop2 can be used in such dual targeting COBRA (pro-drug) constructs or hetero-COBRAs.

### V. Methods of Making Pro-Drug Compositions

The pro-drug compositions of the invention are made as will generally be appreciated by those in the art and outlined below.

The invention provides nucleic acid compositions that encode the pro-drug compositions of the invention.

As is known in the art, the nucleic acids encoding the components of the invention can be incorporated into expression vectors as is known in the art, and depending on the host cells used to produce the prodrug compositions of the invention. Generally ,the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells, 293 cells), finding use in many embodiments. The prodrug compositions of the invention are made by culturing host cells comprising the expression vector(s) as is well known in the art. Once produced, traditional antibody purification steps are done, including an Protein A affinity chromatography step and/or an ion exchange chromatography step.

### VI. Formulation and Administration of the Pro-Drug Compositions

Formulations of the pro-drug compositions used in accordance with the present invention are prepared for storage by mixing the pro-drugs having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions.

The pro-drug compositions of the invention are administered to a subject, in accord with known methods, such as intravenous administration as a bolus or by continuous infusion over a period of time.

The pro-drug compositions provided are useful in the treatment of cancer.

In some embodiments, provided is any one of the pro-drug compositions (e.g., the fusion proteins) described herein for use as a medicament. In some embodiments, provided is any one of the pro-drug compositions (e.g., the fusion proteins) described herein for the treatment of cancer. In some embodiments, provided is any one of the pro-drug compositions (e.g., the fusion proteins) described herein for use in a method treating cancer. In some embodiments, provided is a medicament for treating cancer in a subject, wherein the medicament comprises any one of the pro-drug compositions (e.g., the fusion proteins) described herein. In some embodiments, provided is a pharmaceutical composition comprising any one of the pro-drug compositions (e.g., the fusion proteins) described herein for treating cancer in a subject.

### VII. EXAMPLES

### Example 1: Pro Construct Construction and Purification

Transfections

Each protein was expressed from a separate expression vector (pcdna3.4 derivative). Single chain constructs were transfected to Expi293 cells following the manufacture's transfection protocol. Conditioned media was harvested 5 days post transfection by centrifugation (6000 rpm×25′) and filtration (0.2 uM filter). Protein expression was confirmed by SDS-PAGE. Constructs were purified and the final buffer composition was: 25 mM Citrate, 75 mM Arginine, 75 mM NaCl, 4% Sucrose, pH 7. The final preparations were stored at −80° C.

Activation of MMP9

Recombinant human (rh) MMP9 was activated according to the following protocol. Recombinant human MMP-9 (R&D # 911-MP-010) is at 0.44 mg/ml (4.7 uM). p-aminophenylmercuric acetate (APMA) (Sigma) is prepared at the stock concentration of 100 mM in DMSO, Assay buffer is 50 mM Tris pH 7.5, 10 mM CaCl2, 150 mM NaCl, 0.05% Brij-35.
Dilute rhMMP9 with assay buffer to ˜100 ug/ml (25 ul hMMP9+75 uL assay buffer)
Add p-aminophenylmercuric acetate (APMA) from 100 mM stock in DMSO to a final concentration of 1 mM (1 uL to 100 uL)
Incubate at 37′C for 24 hrs
Dilute MMP9 to 10 ng/ul (add 900 ul of assay buffer to 100 ul of activated solution)

The concentration of the activated rhMMP9 is ˜100 nM.

Cleavage of Constructs for TDCC Assays

To cleave the constructs, 100 ul of the protein sample at 1 mg/ml concentration (10.5 uM) in the formulation buffer (25 mM Citric acid, 75 mM L-arginine, 75 mM NaCl, 4% sucrose) was supplied with CaCl₂ up to 10 mM. Activated rhMMP9 was added to the concentration 20-35 nM. The sample was incubated at room temperature overnight (16-20 hrs). The completeness of cleavage was verified using SDS PAGE (10-20% TG, TG running buffer, 200 v, 1 hr). Samples were typically 98% cleaved.

### Example 2: T Cell Dependent Cellular Cytotoxicity (TDCC) Assays

Firefly Luciferase transduced HT-29 cells were grown to approximately 80% confluency and detached with Versene (0.48 mM EDTA in PBS—Ca-Mg). Cells were centrifuged and resuspended in TDCC media (5% Heat Inactivated FBS in RPMI 1640 with HEPES, GlutaMax, Sodium Pyruvate, Non-essential amino acids, and β-mercaptoethanol). Purified human Pan-T cells were thawed; centrifuged and resuspended in TDCC media.

A coculture of HT-29_Luc cells and T cells was added to 384-well cell culture plates. Serially diluted COBRAs were then added to the coculture and incubated at 37□C for 48 hours. Finally, an equal volume of SteadyGlo luciferase assay reagent was added to the plates and incubated for 20 minutes. The plates were read on the Perkin Elmer Envision with an exposure time of 0.1s/well. Total luminescence was recorded and data were analyzed on GraphPad Prism 7.

### Example 3: General Protocol Design of the in Vivo Adoptive T Cell Transfer Efficacy Model

These protocols were used in many of the experiments of the figures. Tumor cells were implanted subcutaneous (SC) in the right flank of NSG (NOD.Cg-Prkdcscid Il2rgtm1Wjl/SzJ) mice (The Jackson Laboratory, Cat. No. 005557) and allowed to grow until an established tumor with a mean volume of around 200 mm3 was reached. In parallel human T cells were cultured in T cell media (X-VIVO 15 [Lonza, Cat.No. 04-418Q], 5% Human Serum, 1% Penicillin/Streptomycin, 0.01mM 2-Mercaptoethanol) in a G-Rex100M gas permeable flask (Wilson Wolf Cat. No. 81100S) with MACSiBeads from the T Cell Activation/Expansion Kit (Miltenyi Cat. No. 130-091-441) for around 10 days and supplemented with recombinant human IL-2 protein. Tumor growth in mice and human T cell activation/expansion were coordinated so that on Day 0 of the study mice were randomized into groups (N=6) based on tumor size; each were then injected intravenous (IV) with 2.5×10⁶ cultured human T cells and administered the first dose of the COBRA or control molecules. Mice were dosed every 3 days for 7 doses (Days 0, 3, 6, 9, 12, 15 and 18) and then followed for an additional 2-3 weeks until tumors reached >2000 mm3 in volume or the study was terminated. Tumor volumes were measured every 3 days.

### Example 4: Pharmacokinetics Analysis of COBRA-HSA Fusion Proteins

Single dose pharmacokinetic studies were conducted with COBRA-HSA fusion proteins in mice transgenic for human FcRn and deficient for mouse albumin and mouse FcRn (B6.Cg-Tg(FCGRT)32Dcr Alb^{eml2Mmw} Fcgrt^{tmlDcr} Prkdc^{scid}/J) (The Jackson Laboratory, Cat. No. 031644). Further discussion of these mice can be found in Roopenian et al., mAbs, 2015, 7(2): 344-351. These mice were used because half-life extension of albumin is dependent on pH-dependent binding of albumin to the neonatal Fc receptor (FcRn), but human albumin does not bind to mouse FcRn. The COBRA-HSA fusion proteins were administered intravenously (IV) at 0.1 mg/kg, and the concentration of COBRA was subsequently measured in the plasma with a meso scale discovery (MSD) assay. The capture reagent in the MSD assay was biotinylated 13H4, an antibody specific to the anti-CD3 VH sequence in the COBRAs, and the detection reagent was a sulfoTag-labeled anti-Flag antibody. Plasma samples were diluted 1:4 prior to adding to the MSD plate.

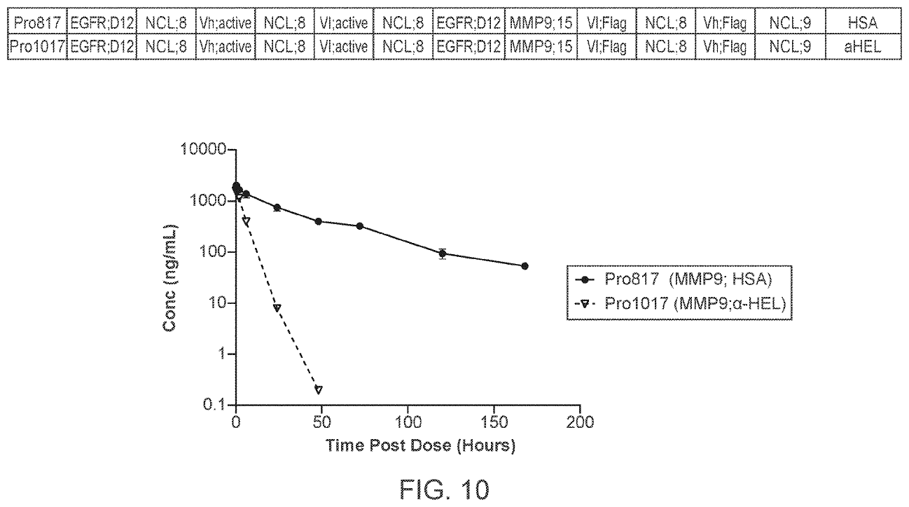

The COBRA plasma concentrations are shown in FIG. 10, and the pharmacokinetic parameters are summarized in Table 2. The half-life of the COBRA-HSA fusion protein Pro817 was approximately 10-fold higher than a COBRA with no half-life extension moiety, Pro1017. Instead of a half-life extension domain, the C-terminus of Pro1017 contains an sdAb specific to hen egg lysozyme (HEL).

TABLE 2
Pharmacokinetic parameters

| | | | | | | CL |
|---|---|---|---|---|---|---|
| | | Dose | T_{1/2} | Cₘₐₓ | AUC | (mL/ |
| Molecule | Format | (mg/kg) | (h) | (ng/ml) | (ng*h/mL) | h/kg) |
| Pro817 | MMP9; HSA | 0.1 | 38.7 | 2023.5 | 64210.4 | 1.54 |
| Pro1017 | MMP9; α-HEL | 0.1 | 3.87 | 1591.2 | 7322.4 | 13.65 |

### Example 5: TDCC Activity of COBRA-MSA Fusion Proteins

To facilitate additional studies in mice, proof-of-concept molecules were constructed as mouse serum albumin (MSA) fusion COBRAs. The pH-dependent interaction between MSA and mouse FcRn results in a half-life for MSA of approximately 35 hours (Chaudhury, J Exp Med, 2003 Feb. 3, 197(3):315-22). Thus, the half-life extension properties of an MSA-fusion COBRA can be evaluated in mice endogenously expressing mouse FcRn.

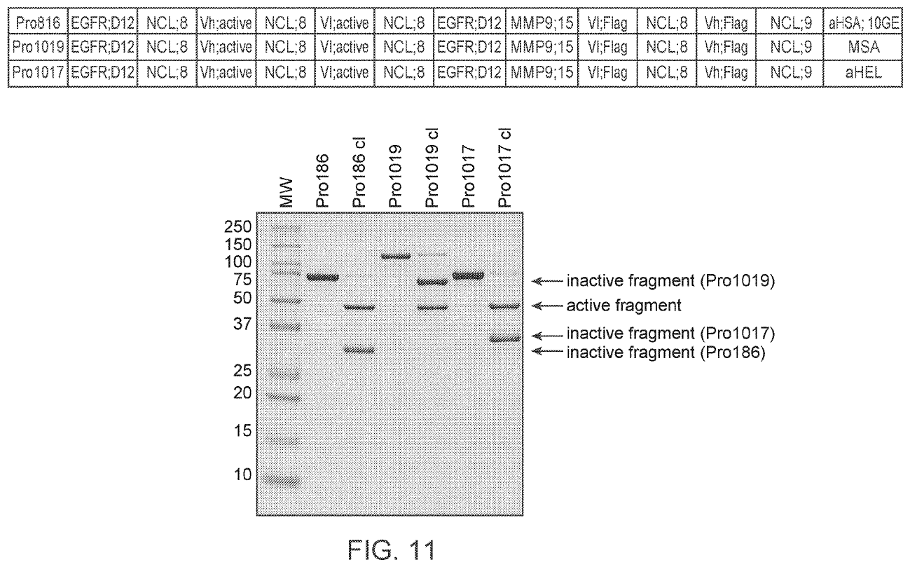

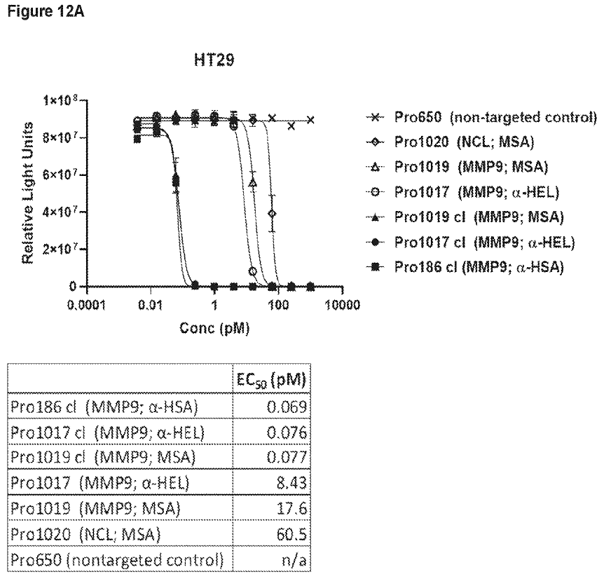

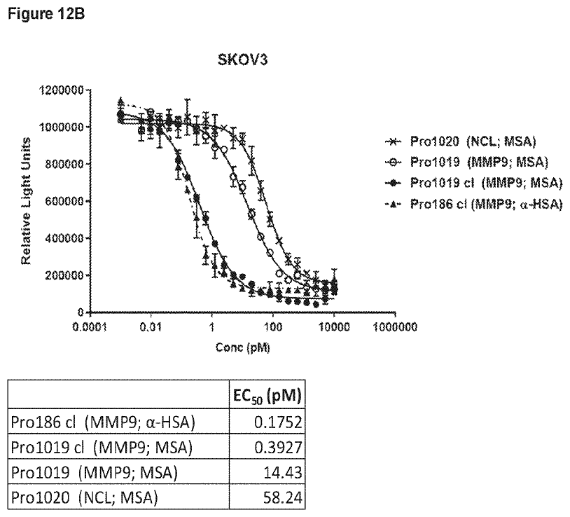

COBRA-MSA fusion proteins were cleaved with MMP9, as described in Example 1, and the products of the cleavage reactions are shown in FIG. 11. The cleaved and uncleaved COBRA-MSA fusion proteins were tested in TDCC assays, run essentially as described in Example 2, except in some cases, the assays were run in 96 well plates. FIGS. 12A-12B depict the results of such TDCC assays, where the COBRA-MSA fusion proteins are shown to be conditionally activated to induce cytotoxicity of tumor cells. In these assays, the potency of cleaved Pro1019, a COBRA-MSA fusion protein, was similar to cleaved Pro186 and to cleaved Pro1017, a COBRA with no half-life extension moiety.

### Example 6: Pharmacokinetics Analysis of COBRA-MSA Fusion Proteins

Single dose pharmacokinetic studies with COBRA-MSA fusion proteins were conducted essentially as described in Example 4, except NOD-SCID mice were used.

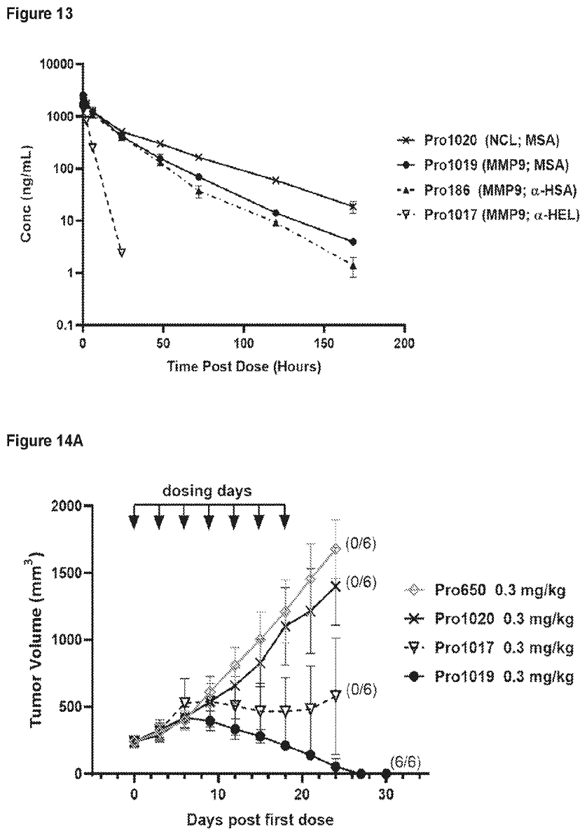

The plasma concentrations of the COBRA-MSA fusion proteins are shown in FIG. 13, and the pharmacokinetic parameters are summarized in Table 3. The half-life of the cleavable COBRA-MSA fusion protein Pro1019 was 23.2 hours, similar to that of Pro186, and approximately 9-fold longer than that of Pro1017, a COBRA protein lacking a half-life extension moiety.

TABLE 3
Pharmacokinetic parameters

| | | | | | | CL |
|---|---|---|---|---|---|---|
| | | Dose | T_{1/2} | Cₘₐₓ | AUC | (mL/ |
| Molecule | Format | (mg/kg) | (h) | (ng/mL) | (ng*h/mL) | h/kg) |
| Pro1019 | MMP9; MSA | 0.1 | 23.2 | 2545.3 | 34521.5 | 2.89 |
| Pro1020 | NCL; MSA | 0.1 | 30.7 | 2264.8 | 46404.7 | 2.12 |
| Pro1017 | MMP9; α-HEL | 0.1 | 2.6 | 1515.2 | 5271.2 | 18.94 |
| Pro186 | MMP9; α-HSA | 0.1 | 20.3 | 1984.6 | 30179.4 | 3.31 |

### Example 7: In Vivo Anti-Tumor Activity of COBRA-MSA Fusion Proteins

COBRA-MSA fusion proteins were evaluated in mice bearing HT29 xenografts, following the protocol outlined in Example 3 above.

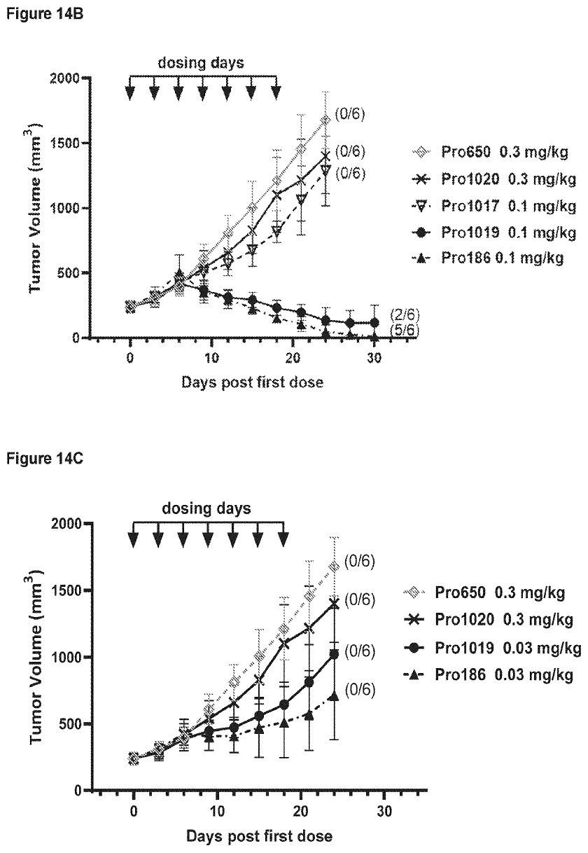

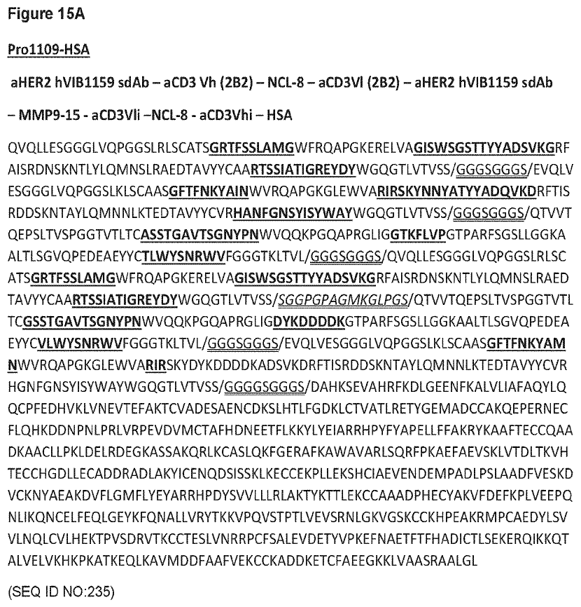

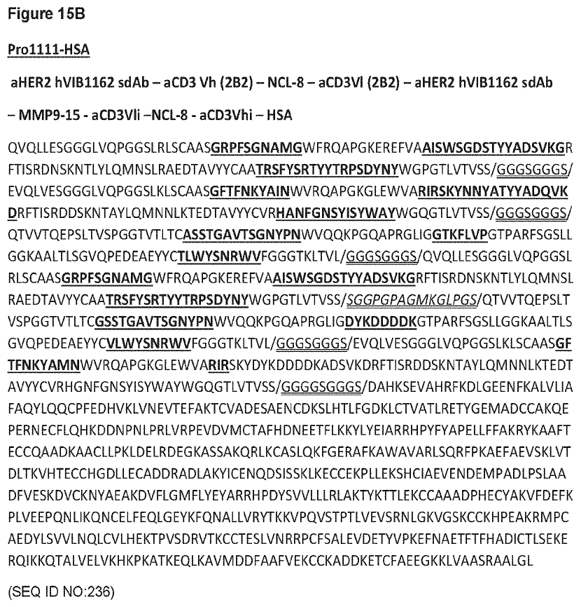

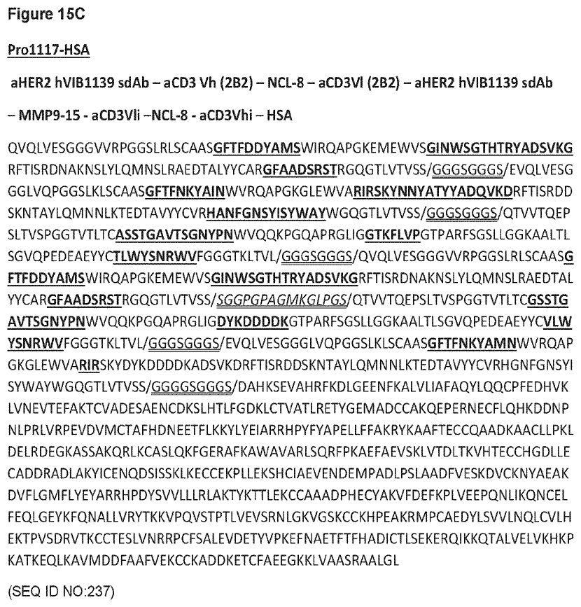

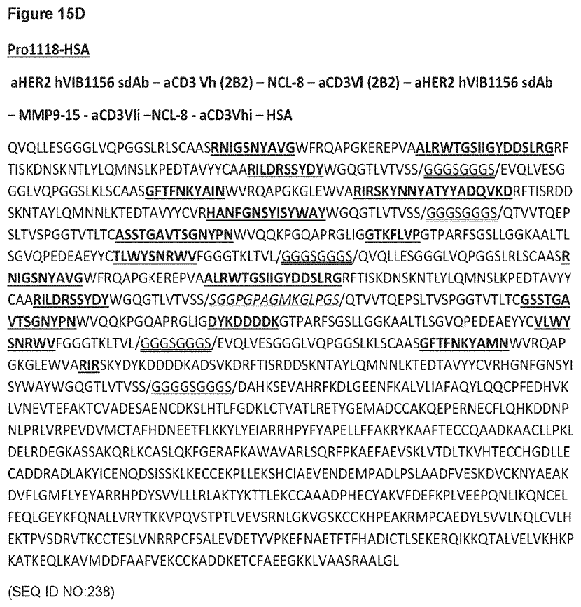

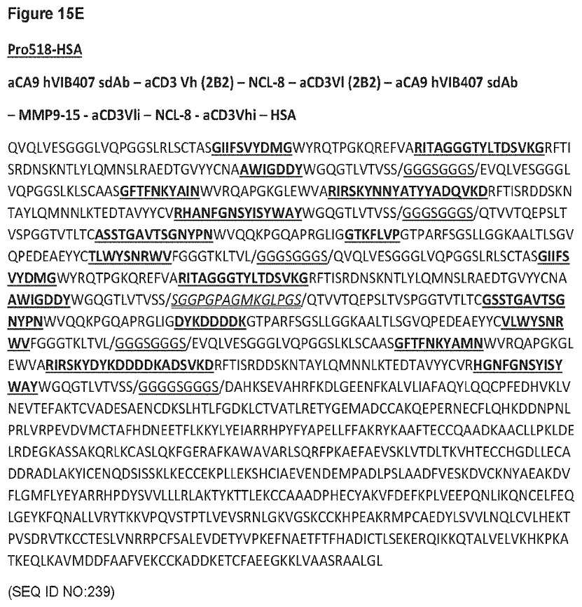

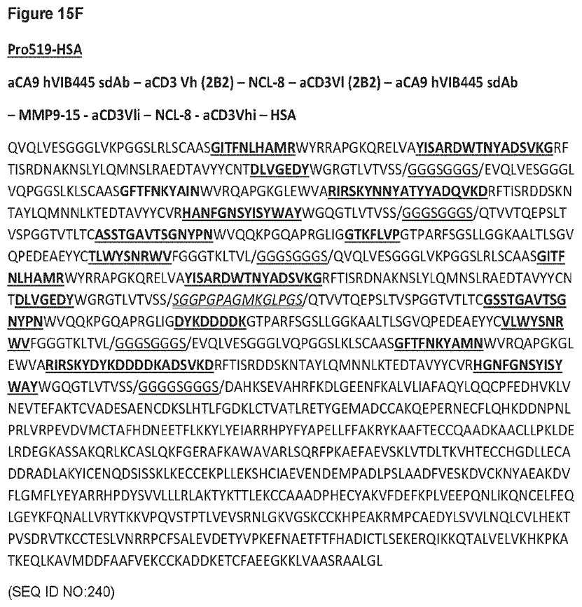

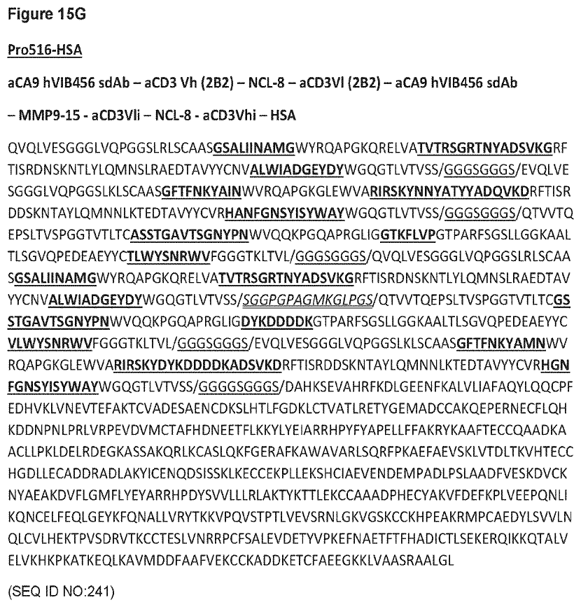

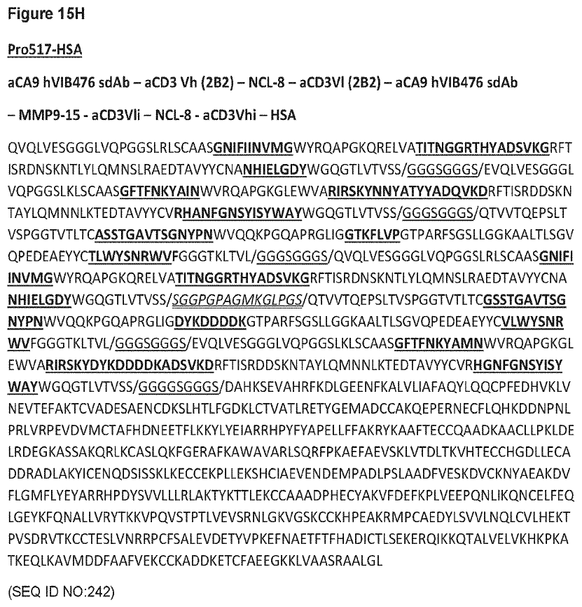

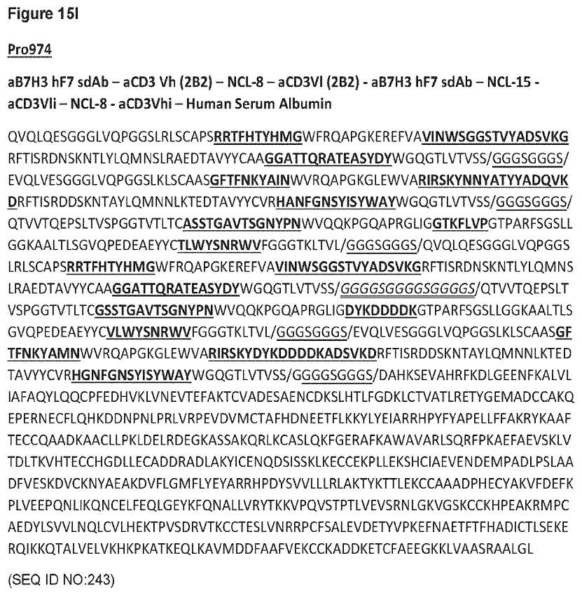

The cleavable COBRA-MSA fusion protein Pro1019 was active at all dose levels tested and showed dose-dependent anti-tumor activity (FIG. 14). Pro1019 treatment at 0.3 mg/kg resulted in complete tumor regression in all animals (FIG. 14A), Pro1019 treatment at 0.1 mg/kg resulted in tumor shrinkage in all animals (FIG. 14B), and Pro1019 treatment at 0.03 mg/kg slowed tumor growth in all animals, compared to the non-targeted COBRA, Pro650, and the non-cleavable COBRA-MSA fusion protein Pro1020 (FIG. 14C). In contrast, administration of Pro1017, with no half-life extension moiety, slowed tumor growth at 0.3 mg/kg (FIG. 14A) and had no significant activity at 0.1 mg/kg (FIG. 14B), compared to the non-targeted COBRA, Pro650 and the non-cleavable COBRA-MSA fusion protein Pro1020. The tumor volumes in Pro1019-treated mice were not significantly different from those in Pro186-treated mice at either the 0.1 mg/kg or 0.03 mg/kg dose levels.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A fusion protein comprising, from N- to C-terminal:
a) a first single domain antigen binding domain (sdABD) that binds to a human tumor target antigen (TTA) (sdABD-TTA);
b) a first domain linker;
c) a constrained Fv domain comprising:
i) a first variable heavy domain comprising a vhCDR1, vhCDR2 and vhCDR3;
ii) a constrained non-cleavable linker (CNCL); and
iii) a first variable light domain comprising v1CDR1, v1CDR2 and v1CDR3;
d) a second domain linker;
e) a second sdABD-TTA;
f) a cleavable linker (CL);
g) a constrained pseudo Fv domain comprising:
i) a first pseudo variable light domain;
ii) a non-cleavable linker (NCL); and
iii) a first pseudo variable heavy domain;
h) a third domain linker; and
i) a human serum albumin (HSA) domain;
wherein the first variable heavy domain and the first variable light domain of the constrained Fv domain are capable of binding human CD3 but the constrained pseudo Fv domain does not bind CD3; wherein the first variable heavy domain and the first pseudo variable light domain intramolecularly associate to form an inactive Fv domain; and wherein the first variable light domain and the first pseudo variable heavy domain intramolecularly associate to form an inactive Fv domain.

2. The fusion protein according to claim 1, wherein the first variable heavy domain is N-terminal to the first variable light domain and the pseudo variable light domain is N-terminal to the pseudo variable heavy domain.

3. The fusion protein according to claim 1, wherein the first variable heavy domain is N-terminal to the first variable light domain and the pseudo variable heavy domain is N-terminal to the pseudo variable light domain.

4. The fusion protein according to claim 1, wherein the first variable light domain is N-terminal to the first variable heavy domain and the pseudo variable light domain is N-terminal to the pseudo variable heavy domain.

5. The fusion protein according to claim 1, wherein the first variable light domain is N-terminal to the first variable heavy domain and the pseudo variable heavy domain is N-terminal to the pseudo variable light domain.

6. The fusion protein according to any one of claims 1-5, wherein the first TTA and the second TTA are the same.

7. The fusion protein according to any one of claims 1-5, wherein the first TTA and the second TTA are different.

8. The fusion protein according to any one of claims 1-7 wherein the first sdABD-TTA and the second sdABD-TTA are the same.

9. The fusion protein according to any one of claims 1-7 wherein the first sdABD-TTA and the second sdABD-TTA are different.

10. The fusion protein according to any one of claims 1-9, wherein the first TTA and the second TTA are selected from the group consisting of B7H3, CA9, EGFR, EpCAM, FOLR1, HER2, LyPD3, Trop2, and any combination thereof.

11. The fusion protein according to any one of claims 1-6 and 8-10, wherein the first TTA and second TTA is B7H3.

12. The fusion protein according to any one of claims 1-6 and 8-10, wherein the first TTA and second TTA is CA9.

13. The fusion protein according to any one of claims 1-6 and 8-10, wherein the first TTA and second TTA is EGFR.

14. The fusion protein according to any one of claims 1-6 and 8-10, wherein the first TTA and second TTA is EpCAM.

15. The fusion protein according to any one of claims 1-6 and 8-10, wherein the first TTA and second TTA is FOLR1.

16. The fusion protein according to any one of claims 1-6 and 8-10, wherein the first TTA and second TTA is HER2.

17. The fusion protein according to any one of claims 1-6 and 8-10, wherein the first TTA and second TTA is LyPD3.

18. The fusion protein according to any one of claims 1-6 and 8-10, wherein the first TTA and second TTA is Trop2.

19. The fusion protein according to any one of claims 1-5, 9 and 10, wherein
(a) the first TTA is B7H3 and the second TTA is selected from the group consisting of B7H3, CA9, EGFR, EpCAM, FOLR1, HER2, LyPD3 and Trop2;
(b) the first TTA is CA9 and the second TTA is selected from the group consisting of B7H3, CA9, EGFR, EpCAM, FOLR1, HER2, LyPD3 and Trop2;
(c) the first TTA is EGFR and the second TTA is selected from the group consisting of B7H3, CA9, EGFR, EpCAM, FOLR1, HER2, LyPD3 and Trop2;
(d) the first TTA is EpCAM and the second TTA is selected from the group consisting of B7H3, CA9, EGFR, EpCAM, FOLR1, HER2, LyPD3 and Trop2;
(e) the first TTA is FOLR1 and the second TTA is selected from the group consisting of B7H3, CA9, EGFR, EpCAM, FOLR1, HER2, LyPD3 and Trop2;
(f) the first TTA is HER2 and the second TTA is selected from the group consisting of B7H3, CA9, EGFR, EpCAM, FOLR1, HER2, LyPD3 and Trop2;
(g) the first TTA is LyPD3 and the second TTA is selected from the group consisting of B7H3, CA9, EGFR, EpCAM, FOLR1, HER2, LyPD3 and Trop2; or
(h) the first TTA is Trop2 and the second TTA is selected from the group consisting of B7H3, CA9, EGFR, EpCAM, FOLR1, HER2, LyPD3 and Trop2.

20. The fusion protein according to any one of claims 1-5, 9 and 10, wherein
(a) the first TTA is selected from the group consisting of B7H3, CA9, EGFR, EpCAM, FOLR1, HER2, LyPD3 and Trop2 and the second TTA is B7H3;
(b) the first TTA is selected from the group consisting of B7H3, CA9, EGFR, EpCAM, FOLR1, HER2, LyPD3 and Trop2 and the second TTA is CA9;
(c) the first TTA is selected from the group consisting of B7H3, CA9, EGFR, EpCAM, FOLR1, HER2, LyPD3 and Trop2 and the second TTA is EGFR;
(d) the first TTA is selected from the group consisting of B7H3, CA9, EGFR, EpCAM, FOLR1, HER2, LyPD3 and Trop2 and the second TTA is EpCAM;
(e) the first TTA is selected from the group consisting of B7H3, CA9, EGFR, EpCAM, FOLR1, HER2, LyPD3 and Trop2 and the second TTA is FOLR1;
(f) the first TTA is selected from the group consisting of B7H3, CA9, EGFR, EpCAM, FOLR1, HER2, LyPD3 and Trop2 and the second TTA is HER2;
(g) the first TTA is selected from the group consisting of B7H3, CA9, EGFR, EpCAM, FOLR1, HER2, LyPD3 and Trop2 and the second TTA is LyPD3; or
(h) the first TTA is selected from the group consisting of B7H3, CA9, EGFR, EpCAM, FOLR1, HER2, LyPD3 and Trop2 and the second TTA is Trop2.

21. The fusion protein according to any of claims 1-22, wherein the first and/or second sdABD-TTAs are selected from the group consisting of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:258, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:308, SEQ ID NO:312, SEQ ID NO:316, SEQ ID NO:320, SEQ ID NO:324, SEQ ID NO:328, SEQ ID NO:332, SEQ ID NO:336, SEQ ID NO:340, SEQ ID NO:344, and any combination thereof.

22. The fusion protein according to any one of claims 1-21, wherein the HSA domain comprises an amino acid sequence having at least 90% sequence identity to SEQ ID NO:117.

23. The fusion protein according to any one of claims 1-22, wherein the HSA domain comprises the amino acid sequence of SEQ ID NO:117.

24. The fusion protein according to any one of claims 1-23, wherein the cleavable linker is cleaved by a human protease selected from the group consisting of MMP2, MMP9, meprin A, meprin B, cathepsin S, cathepsin K, cathespin L, granzymeB, uPA, kallekriein7, matriptase and thrombin.

25. The fusion protein according to any one of claims 1-24, wherein the cleavable linker comprises an amino acid sequence selected from the group consisting of SEQ ID NOS:152-225.

26. The fusion protein according to any one of claims 1-25, wherein the domain linker is a flexible linker.

27. The fusion protein according to claim 26, wherein the flexible linker comprises an amino acid sequence selected from the group consisting of (GS)n, (GGS)n, (GGGS)n (SEQ ID NO:244), (GGSG)n (SEQ ID NO:245), (GGSGG)n (SEQ ID NO:246), or (GGGGS)n (SEQ ID NO:247), wherein n is 1, 2, 3, 4, 5, 6, 7, 8, 9, or 10.

28. The fusion protein according to any one of claims 1-27, wherein the first variable heavy domain comprises a vhCDR1 of SEQ ID NO:135, a vhCDR2 of SEQ ID NO:136 and a vhCDR3 of SEQ ID NO:137.

29. The fusion protein according to any one of claims 1-28, wherein the first variable light domain comprises a v1CDR1 of SEQ ID NO:119, a v1CDR2 of SEQ ID NO:120 and a v1CDR3 of SEQ ID NO:121.

30. The fusion protein according to any one of claims 1-29, wherein the first variable heavy domain comprises the amino acid sequence of SEQ ID NO:134 and the first variable light domain comprises the amino acid sequence of SEQ ID NO:118.

31. The fusion protein according to any one of claims 1-30, wherein the constrained pseudo Fv domain comprises the first pseudo variable light domain having the amino acid sequence of SEQ ID NO:122 and the first pseudo variable heavy domain having the amino acid sequence of SEQ ID NO:138.

32. The fusion protein according to any one of claims 1-30, wherein the constrained pseudo Fv domain comprises the first pseudo variable light domain having the amino acid sequence of SEQ ID NO:126 and the first pseudo variable heavy domain having the amino acid sequence of SEQ ID NO:142.

33. The fusion protein according to any one of claims 1-30, wherein the constrained pseudo Fv domain comprises the first pseudo variable light domain having the amino acid sequence of SEQ ID NO:130 and the first pseudo variable heavy domain having the amino acid sequence of SEQ ID NO:146.

34. The fusion protein according to any one of claims 1-33, having an amino acid sequence selected from the group consisting of SEQ ID NOS:226-231 and 235-243.

35. The fusion protein according to any one of claims 1-34, having at least a 10%, 20%, 30%, 40%, 50%, 60%, 70%, 80%, or 90% increase in serum half-life relative to a corresponding fusion protein without a half-life extension domain.

36. The fusion protein according to any one of claims 1-35, having at least a 100%, 200%, 300%, 400%, 500%, 600%, 700%, 800%, or 900% increase in serum half-life relative to a corresponding fusion protein without a half-life extension domain.

37. The fusion protein according to any one of claims 1-36, having at least a 1000% increase in serum half-life relative to a corresponding fusion protein without a half-life extension domain.

38. The fusion protein according to any one of claims 35-37, wherein the increase in serum half-life is determined using a mouse surrogate for evaluating pharmacokinetics of a human serum albumin domain.

39. The fusion protein according to claim 38, wherein the mouse surrogate is an Alb' hFcRn humanized mouse.

40. The fusion protein according to claim 39, wherein the Alb- hFcRn humanized mouse is a Tg32-Alb^{-/-} mFcRn^{-/-} hFcRn^{Tg/Tg} mouse.

41. A nucleic acid encoding a fusion protein according to any one of claims 1-40.

42. An expression vector comprising the nucleic acid of claim 41.

43. A host cell comprising the expression vector of claim 41

44. A method of making a fusion protein comprising (a) culturing the host cell of claim 43 under conditions wherein the fusion protein is expressed and (b) recovering the fusion protein.

45. A method of treating cancer comprising administering the fusion protein of any one of claims 1-40 to a subject.

46. Use of the fusion protein of any one of claims 1-40 in the manufacture of a medicament for the treatment of cancer.
